(19) United States
(12) Patent Application Publication
(10) Pub. No.: US 2025/0051762 A1
(43) Pub. Date: Feb. 13, 2025

(54) PLATFORM USING DNA-ENCODED SMALL MOLECULE LIBRARIES AND RNA SELECTION TO DESIGN SMALL MOLECULES THAT TARGET RNA

(71) Applicant: University of Florida Research Foundation, Incorporated, Gainesville, FL (US)

(72) Inventors: Matthew D. Disney, Gainesville, FL (US); Brian M. Paegel, Gainesville, FL (US)

(73) Assignee: University of Florida Research, Gainesville, FL (US)

(21) Appl. No.: 18/717,720

(22) PCT Filed: Dec. 8, 2022
(86) PCT No.: PCT/US2022/081215

§ 371 Date: Jun. 7, 2024

Related U.S. Application Data
(60) Provisional application No. 63/287,814, filed on Dec. 9, 2021.

(51) Int. Cl. C12N 15/10; C12Q 1/6806; C12Q 1/6834; C12Q 1/6869; G01N 21/64

(52) U.S. Cl. CPC C12N 15/1093; C12Q 1/6806; C12Q 1/6834; C12Q 1/6869; G01N 21/6486; C12Q 2600/178

(57) ABSTRACT

A dual screen technique using DEL and 2DCS screens with libraries of synthesized small molecules and synthesized RNA structures (4,096 targets) delivers bona fide ligand-RNA 3D fold target pairs. One of the newly discovered ligands bound a 5′GAG/3′CCC internal loop that is present in pri-miR-27a, the oncogenic precursor of microRNA-27a. The DEL-derived pri-miR-27a ligand is cell-active, potently and selectively inhibits pri-miR-27a processing to reprogram gene expression and halt an otherwise invasive phenotype in triple-negative breast cancer cells.

### CROSS-REFERENCE TO RELATED APPLICATIONS

This application claims the benefit under 35 U.S.C. § 119 (c) of U.S. provisional application No. 63/287,814, filed Dec. 9, 2021, the entire contents of which are incorporated herein by reference.

### STATEMENT OF GOVERNMENT SUPPORT

This invention was made with government support under Contract Numbers CA249180, GM120491 and GM140890 awarded by the National Institutes of Health. The government has certain rights in the invention.

### REFERENCE TO AN ELECTRONIC SEQUENCE LISTING

The contents of the electronic sequence listing (U120270113WO00-SEQ-JDH.xml; Size: 108,466 bytes; and Date of Creation: Dec. 8, 2022) is herein incorporated by reference in its entirety.

### BACKGROUND

Traditional drug discovery entails screening a single or a few targets for binding to a library of small molecules. (1) The dominant high-throughput screening paradigm is responsible for generating countless new lead molecules. These screening data sets and the accompanying compound library files guide medicinal chemistry by way of off-target effects gleaned from prior screens and deep physicochemical and pharmacokinetic analyses that all library members undergo, ultimately focusing the library on “drug-like” chemical matter. (2, 3) High-throughput screens are limited to the same “drug-like” chemical matter, (4, 5) leading to the classification of a target as “druggable” or “undruggable”. However, the screened chemical matter, developed for “druggable” protein and enzyme targets, is not appropriate for all potential target classes.

Genetic encoding of targets and small molecules has greatly increased both the scope of library property space and the information content obtained from such screens. (6-10) DNA-encoded library (DEL) technology permits the design and synthesis of highly diverse (˜10⁶) collections of small molecules, each bound to a structure-encoding DNA tag (6, 7, 11-14). Affinity selection and sequencing of the bound species' DNA tags affords potent ligands and rich structure-activity data. (15, 16)

RNA sequencing technology has become a powerful tool for identifying selective RNA ligands. (17-21) For example, two-dimensional combinatorial screening (2DCS) presents a library of RNA 3D folds to a known small molecule microarray. This 2DCS technique allows thousands of compounds to be screened for binding to thousands of RNA targets, but the disadvantage is that each compound must be individually identified and assayed from the bound RNA library in order to determine the bound compounds.

Therefore, integration of the 2DCS and DEL screening techniques can provide a massively parallel screening pipeline to probe affinity landscapes between RNA folds and small molecules. This integration can enable development of new microRNA binding compounds from diverse libraries not already identified as RNA binding compounds.

### SUMMARY OF THE INVENTION

The present invention is directed to a novel approach to cross reference combinatorially synthesized chemical matter with RNA 3D fold space. Deep sequencing of both the DNA-encoded ligands and the RNA targets proved transformative in the same manner that sequencing has enabling technologies such as ribosome profiling (54, 55) and cell Atlasing (56), which provide high-definition summaries of cellular metabolism and fate. (57) This approach integrates two orthogonal and yet highly complementary library analyses, deploying evolutionary principles (58-61) at the earliest stage of probe discovery. Screening hits are identified with confidence by pairing redundant hit isolation in FACS (29) with in vitro selection-based RNA motif ranking by sequence abundance. (22)

The present invention presents a new capability for DEL screening in the way of RNA library interrogation. Nucleic acid binding protein and nucleic acid targets have long been eschewed due to problems of DEL non-specific binding to other nucleic acids. Embodiments of the invention involve simultaneously decoupling the DNA encoding tag and encoded library member through sub-stoichiometric functionalization of each DEL bead (15) and leverage dual-channel simultaneous counter screening to triage non-selective RNA ligands by way of the base-paired control. This strategy is also highly effective in identifying selective IgG ligands from plasma. (24) Radiometric and high-throughput 2DCS-based RNA simultaneously affords an orthogonal mode of validation and an added layer of statistical hypothesis testing in the form of RNA target abundance analysis of the selection.

The screening approach according to the invention establishes a compelling platform for evaluating novel and designed chemical matter (e.g., compounds defined herein as organic compounds or small molecules) for its suitability to target RNA, a major ongoing initiative in the field of RNA-targeted drug discovery. This DEL-2DCS approach makes it possible to design and synthesize arbitrarily diverse libraries for screening and potentially map whole chemical spaces and their proclivity to bind RNA 3D folds in addition to mapping specific small molecule structures to transcriptomic binding sites of interest as in this study.

According to the present invention, microRNA (miRNA) binding compounds can be developed from diverse libraries of compounds not already known for nucleotide binding properties. An embodiment of the present invention may be developed through use of fluorescence-activated cell sorting (FACS) identified DEL beads that specifically bind to RNA folds. Peptide compounds can be isolated in a FACS analysis of DEL beads (73,728 members) that are incubated with two differentially labeled RNA libraries: i) a library displaying the randomized region in a 3×3 nucleotide internal loop pattern (3×3 ILL; 4,096 members) and ii) a fully base-paired RNA counter screen target. Sequencing and informatic analysis reveal affinity landscapes and candidate target miRNAs for each newly discovered ligand.

Pursuant to the combination of DEL and 2DCS multi-dimensional screening techniques, a group of peptide compounds having strong affinity for oncogenic primary microRNA-27a (pri-miR-27a) and for microRNA-409 have been obtained. Study of the affinities between pri-miR27a and this group of peptide compounds, three peptide compounds with significantly strong selective affinity for pri-miR-27A were identified and the one peptide compound with the strongest selective affinity pri-miR-27a was selected for further examination according to the invention. This peptide compound described herein as pd9S diastereomer is shown to have nanomolar affinity for pri-miR-27A and is shown to inhibit miRNA biogenesis and rescue a migratory phenotype in triple negative breast cancer (TNBC) cells.

The group of peptide compounds demonstrating an ability to selectively target microRNA-27A or microRNA-409 has Formula I:

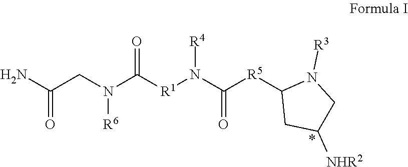

The substituent R⁶ of Formula I is hydrogen, C1-C3 alkyl or propargyl. The propargyl group enables binding to microwell a micro-affinity array through the coupling of the triple bond with an azide to form a triazole ring. The propargyl group is not a part of the targeting peptide compounds apart from its use with the microaffinity array.

The substituent R¹ is —CHR⁷— with R⁷ being phenyl, butyl, isobutyl, 2-cyanophenyl, CH₂CH (CH₂(3-indolyl)). R⁴ is hydrogen. Alternatively, R⁴ together with R⁷ form a —CH₂—CH₂-group bound to the CH of R¹ and N to form an azetidine ring.

Additional R substituents are defined as follows. The substituent R⁵ is a bond between carbonyl and the pyrrolidine ring or is CHR⁸NHCO— wherein R⁸ and R⁴ together form a pyrrolidinone ring provided that R⁴ cannot be both of the azetidine ring and the pyrrolidinone ring. The substituent R³ is hydrogen or —CO—R⁹ and R⁹ is 5-trifluoromethylbenzothiophen-2-yl, 5-chloroindol-3-yl or 2-(4-morpholinylcarbonyl) phenyl provided that R³ is hydrogen when R¹ is CHR⁷ and R⁷ is 2-cyanophenyl or CH₂CH (CH₂(3-indolyl)). The substituent R² is hydrogen or COR¹⁰ and R¹⁰ is tetrahydrofuran-2-yl, or quinazolindion-3-ylmethylenyl, provided that R² is not hydrogen when R³ is hydrogen. The asterisk of Formula I indicates the position of the diastereomeric R and S forms of Formula I.

The five individual peptide compounds of Formula I which display the affinity binding described above include Formula pc1 R and S diastereomers, Formula pc1(S), pc2 (R) diastereomers, Formula pc3(S), pc4(R) diastereomers, Formula pc5(S), pc6(R) diastereomers, Formula pc7(S), pc8(R) diastereomers and/or Formula pc9(S), pc10(R) diastereomers.

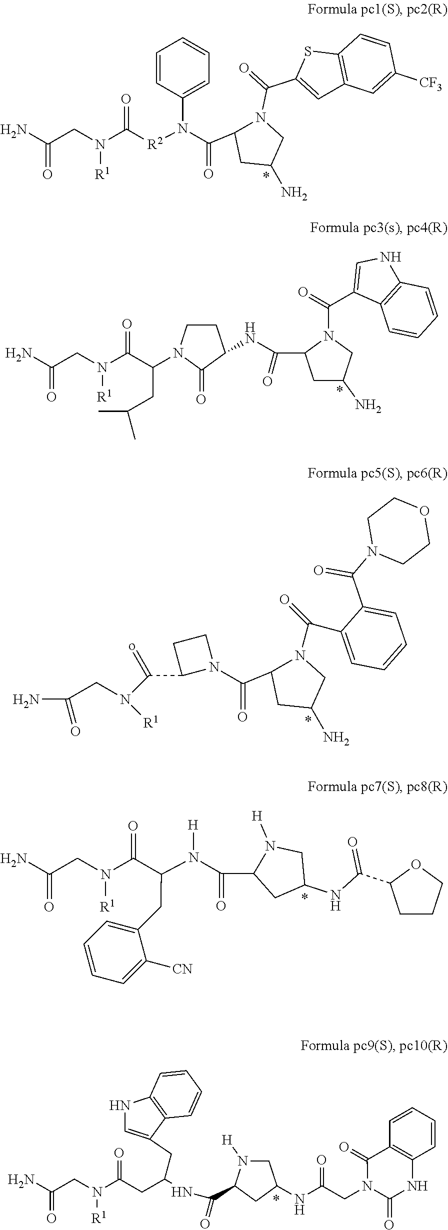

Of these, the S diastereomers of Formulas pc5(S), pc7(S) and pc9(S) are preferred and the S diastereomer of Formula pc9(S) is especially preferred.

Methods according to the present invention include targeting 286 and/or microRNA-409 with a peptide compound of Formula I. The binding inhibits and/or suppresses Drosha nuclease activity upon pri-miR-27a and microRNA-409. The targeting selectively binds the peptide compound with pri-miR-27a and/or microRNA-409 and pc5(S), pc7(S) and pc9(S) selectively bind pri-miR-27a Drosha processing site 5′GAG/3′CCC but does not bind the adjacent 5CAG/3′GCC site.

The peptide compound of Formula pd9(S) diastereomer inhibits and/or suppresses pri-miR-27a activity in MCA-MD-231 cells, MCR-7 cells, LNCaP cells and HeLa cells in cell culture and when these cell lines are present in animals.

A pharmaceutical composition of the peptide compound of Formula pd9(S) diastereomer and a pharmaceutically acceptable carrier may be administered to such an animal for treatment of abnormal activity of such cell lines.

### BRIEF DESCRIPTION OF FIGURES

FIGS. 1A-1D depict schematics showing how RNA-binding ligands are identified using a DNA-encoded library (DEL) and two-dimensional combinatorial screening (2DCS). 1A: A schematic of the solid-phase DNA-encoded library (73,728 unique compounds) screened for binding to a DY647-labeled RNA library with a randomized region in a 3×3 nucleotide internal loop pattern, DY647-3×3 ILL (4,096 unique RNA 3D folds) (SEQ ID NO: 283). The screen was completed in the presence of an orthogonally labeled base-paired control RNA, TAMRA-BP. The DEL was designed to contain molecules with RNA-binding or “antibacterial-like” properties. 1B: sorting of two-color FACS identified beads that specifically bound DY647-3×3 ILL. 1C: shows the FACS histogram collection gate (cyan) highlights beads that selectively bound DY647-3×3 ILL. 1D: the schematic for DNA tag identification of the hit structures that bound selectively to DY647-3×3 ILL library by sequencing their DNA tags (SEQ ID NO: 310).

FIGS. 2A-2C illustrate 2DCS selection to generate transcriptome-wide structure-activity relationships across the human miRNome. 2A: the several structural families were discovered after the 2DCS selection process. 2B: a schematic representation of the construction of 2DCS microarrays, representative microarray image of the 2DCS selection of hit compounds with ³²P-labeled 3×3 ILL, and the affinity landscapes for 5, 7, and 9 (pc5S, pc7S, pc9S, hereinafter 5, 7 and 9). All three compounds bound the 5′GAG/3′CCC internal loop present in pri-miR-27a's Drosha processing site. 2C: a schematic of how the transcriptome-wide mining analysis identified pri-miR-27a as a druggable RNA target, containing the 5′GAG/3′CCC internal loop in its Drosha processing site (SEQ ID NOs: 284-286). Mature miR-27 regulates the expression of PDCD4 and PP4C, the repression of which contributes to migration and oncogenicity.

FIGS. 3A-3D present bar graphs demonstrating the experimental results showing that compound 9 (pd9 (s)) inhibits the biogenesis of pri-miR-27a in MCF-10a (forced expression) and MDA-MB-231 (endogenous) cells. Error bars are reported as SD for all panels. *, P<0.05; **P, <0.01; ***, P<0.001; ****, P<0.0001, as determined by a one-way ANOVA relative to “0” (untreated cells). 3A: the secondary structure of wild type pri-miR-27a contains tandem 9 binding sites (SEQ ID NO: 287). Wild type primary and mature miR-27a expression levels in transfected MCF-10a cells changed significantly and, in a dose-dependent fashion upon treatment with 9 (RT-qPCR, n=3). MCF-10a cells do not otherwise appreciably express miR-27a (Ct >31). “Mock” indicates mock transfected, vehicle-treated cells. 3B: the binding sites for 9 were eliminated in a synthetic base-paired mutant pri-miR-27a (SEQ ID NO: 288). Base-paired mutant primary and mature miR-27a expression levels in transfected MCF-10a cells did not change in response to treatment with 9 (RT-1PCR, n=3). “Mock” indicates mock transfected, vehicle-treated cells. 3C: a bar graph showing how endogenous pri-miR-27a biogenesis and mature miR-27a accumulation in MDA-MB-231 TNBC cells increased and decreased, respectively, in a dose-dependent fashion (RT-qPCR, n=3). 3D: a distribution graph showing that miRNome analysis of MDA-MB-231 cells treated with 9 (1 μM) revealed highly selective attenuation of mature miR-27a levels (FDR, false discovery rate).

FIGS. 4A-4C show the Effect of 9 proteome-wide and on the migratory nature of MDA-MB-231 cells. 4A: a graph of the proteomic analysis of MDA-MB-231 cells treated with 9 (pc9) (LC-MS/MS, fold change relative to vehicle treatment) confirmed the increased abundance of proteins under regulation of miR-27a expression (red squares; n=3). Dotted lines indicate FDR=5% and S0 of 0.1, where S0 is the minimum fold change required for significance; adjusted p=0.05. 4B: a graph of cumulative distribution plots of the fold change of proteins in 9 (pc9)-treated vs. vehicle-treated samples indicated a significant upregulation of only miR-27a targets (red), while no significant change was observed with miR-23a targets (green) and miR-24 (blue), relative to the cumulative distribution of all proteins (black). TargetScanHuman v7.2 was used to predict downstream protein targets whose mRNAs contain conserved sites for miR-27a-3p (n=1421), miR-23a-3p (n=247), and miR-24-3p (n=1342). Expression levels of miR-27a-5p, miR-23a-5p, and miR-24-5p are very low in MDA-MB-231 cells (Cₜ˜ 28-32).] Of these predicted targets, 220 downstream targets of miR-27 were detectable in proteomics analysis (˜15%), while 247 (˜18%) and 122 (˜16%) were detectable for miR-23a, and miR-24, respectively. Note miR-27a is transcribed as part of a cluster with miR-23a and miR-24. P-values between distributions were calculated using a two-tailed Kolmogorov-Smirnov test. 4C: representative micrographs capture the reduction in migrated MDA-MB-231 cells as a function of 9 (pc9). Image analysis indicated dose-dependent attenuation of the migration phenotype normalized to vehicle-treated cells. Error bars are reported as SD; n=3 replicates from 2 independent experiments; 3 fields of view analyzed per sample. ***, P<0.001; ****, P<0.0001, as determined by a one-way ANOVA relative to “vehicle”.

FIG. 5 provides a schematic of the DEL combinatorial synthesis.

FIG. 6 illustrates a schematic of the synthesis of peptide hit compounds on solid support.

FIGS. 7A-7C display SAR plots of molecular properties of all approved CNS-active drugs and antibacterials. The CNS plots are dark gray. The antibacterial plots are light gray. 6A: polar surface area. 6B: rotatable bonds. 6C: hydrogen bond donors. 6D: hydrogen bond acceptors.

FIGS. 8A-8D display SAR plots of molecular properties of DEL1 drugs targeting RNA and DEL2 antibacterials targeting RNA. The DELIs are dark gray. The DEL2s are light gray. 8A: polar surface area. 8B: rotatable bonds. 8C: hydrogen bond donors. 8D: hydrogen bond acceptors.

FIGS. 9A-9D depict gating strategy for 2-color FACS analysis of the binding of SPDEL to an RNA motif library labeled with DY647 and a control, fully paired RNA labeled with TAMRA. The graphs plot relative fluorescence units (RFU) at 582 nm and 670 nm vs. side scatter (SSC), respectively. 9A: the TAMRA control FACS analysis. 9B: the motif library labeled with DY647. 9C: the TAMRA− and TAMRA+analysis. 9D: the DY647− and DY647+analysis.

FIG. 10 depicts the structure deconvolution of the DNA tags from DEL compound beads that bind 3×3 ILL selectively, as determined by Next-Generation Sequencing (NGS).

FIG. 11 plots the structural similarities of hit compounds to previously identified RNA-binding small molecules housed in Inforna, as determined by Tanimoto coefficients. Heat map of the Tanimoto scores calculated for 1-10 as compared to small molecules in Inforna. (1) a database of all known RNA-binding small molecules reported in the literature by our laboratory and others (SEQ ID NO: 295). The average similarity of the DEL hit compounds compared to Inforna small molecules is 0.3+0.01.

FIG. 12 plots affinity landscapes for compounds 1-10 and the motifs in miRNA Drosha and Dicer processing sites that they bind. Affinity landscapes were generated by plotting the RNA motif's rank in the sequencing data as a function of Z_{obs}. RNA 3D folds present in disease-associated miRNAs are highlighted.

FIG. 13 shows plots correlating binding affinity of compound 9 and Z_{obs}, for the 3×3 random RNA loop motifs. Top: a summary plot of binding affinities of 9 for RNA 3D folds as a function of Z_{obs}, measured via competitive MST binding assay using the Cy5-labeled RNA model of miR-27a's Drosha site (FIGS. 3 and 17). Competitive dissociation constants (K_{d}) are reported as average and standard deviation from two independent experiments. Bottom: representative MST binding plots for 9 and individual 3×3 RNA loop motifs with a range of Z_{obs} (SEQ ID NOs: 289-291).

FIG. 14 is a plot showing the number of RNA 3D folds that bind each small molecule with Z_{obs} >4, as determined by Hit-StARTS analysis of the RNA-seq data used to deconvolute the 2DCS selection.

FIGS. 15A-15C provide a LOGOS analysis (2) for the RNA 3D folds with the top 0.5% of Z_{obs} score for each compound, and each nucleotide preference in the randomized region reported as bits. DiffLOGO analysis (3) was also completed on diastereomer pairs and are shown beneath the corresponding LOGOS. The secondary structures of the RNA 3D folds with the four highest Z_{obs} scores are also shown. 15A: a LOGOS analysis of preferred RNA 3D folds for compounds 1, 2, 3 and 4 (pc1S, pc2R, pc3S, pc4R) (SEQ ID NO: 292). 15B: a LOGOS analysis of preferred RNA 3d folds for compounds 5, 6, 7, 8, 9 and 10 (pc5S, pc6R, pc7S, pc8R, pc9S and pc10R) (SEQ ID NO 292). 15C: the SEQ ID NOs for the RNA 3d folds of FIGS. 15A and 15B (SEQ ID NO: 294).

FIG. 16 provides the secondary structures of miRNAs containing 5′GAG/3′CCC internal loops in functional (top) and non-functional (bottom) sites (SEQ ID NOs: 295-303). The SEQ ID NOs of the named miRNA's with loops of FIG. 16 are as follows: miRNA hsa-miR-27a is SEQ ID NO:243. miRNA hsa-miR-409 is SEQ ID NO:244. miRNA hsa-miR-589 is SEQ ID NO:245. miRNA hsa-miR-1249 is SEQ ID NO:246. miRNA hsa-miR-193a is SEQ ID NO:247. miRNA has-miR-606 is SEQ ID NO:248. miRNA hsa-miR-532 is SEQ ID NO:249. miRNA hsa-miR-940 is SEQ ID NO:250. miRNA hsa-miR-1249 is SEQ ID NO: 251.

FIG. 17 depicts representative binding affinity curves for 5, 7, 9, and 10 for the 3D fold in pri-miR-27a's Drosha processing site and for control RNAs SEQ ID NOs: 304-306). Binding affinities were measured by microscale thermophoresis (MST). Error is reported as SD, and the affinity of each interaction is the average of at least 3 independent experiments. mRNA pri-miR-27A precursor depicted on FIG. 17 has SEQ ID NO:252

FIGS. 18A-18D depict the affinity of 9 for pri-miR-27a and a mutant pri-miR-27a. as determined by a competitive binding assay. 18A: the secondary structure of the pri-miR-27a RNA used in a competitive binding experiment with a Cy5-labeled model of its Drosha binding site, SEQ ID NOs: 253 and 295). 18B: the representative binding curve for the competitive binding experiment between 9, unlabeled pri-miR-27a, and the Cy5-labeled WT Drosha site (SEQ ID NO: 307). 18C: the secondary structure of the mutant pri-miR-27a RNA used in a competitive binding experiment with a Cy5-labeled model of WT pri-miR-27a's Drosha binding site, SEQ ID NO: 254. 18D: the representative binding curve for the competitive binding experiment between 9, unlabeled mutated pri-miR-27a, and the Cy5-labeled Drosha site. Error is reported as SD, and Kas are reported as the average of 2 independent experiments.

FIGS. 19A-19B illustrate that pri-miR-27a induces a migratory phenotype in MCF-10a, a cellular model of healthy breast epithelium, that is rescued by 9. 19A: representative microscopic images of the migratory phenotype induced by WT pri-miR-27a and mutant pri-miR-27a in MCF-10a cells and effect of 9-treatment. Compound 9 only reduced migration of MCF-10a cells that express WT pri-miR-27a as its binding site is abolished in the mutant pri-miR-27a. 19B: a graph plot of the quantification of the number of migratory cells with or without treatment from microscopic images, relative to vehicle-treated samples. Error bars are reported as SD; n=3 replicates from 2 independent experiments; 3 fields of view analyzed per sample. ***, P<0.001; ****, P<0.0001, as determined by Student t-test compared to “vehicle”.

FIGS. 20A-20D demonstrate validation of RT-qPCR primers used to measure levels of pri-miR-27a and housekeeping (control) genes GAPDH and 18S by RT-qPCR. 20A: plots of Cₜ values as a function of cDNA dilutions from reverse transcription. 20B: melting curves of RT-qPCR products, supporting that only one species is amplified for each gene. 20C: plots showing that no template control does not amplify by RT-qPCR (C, values >32). 20D: a gel analysis of RT-qPCR product and shows that only one band (species) is generated.

FIGS. 21A-21D demonstrate validation of RT-qPCR primers used to measure levels of mature miR-27a and housekeeping (control) gene U6 by RT-qPCR. 21A: plots of Cₜ values as a function of cDNA dilutions from reverse transcription. 21B: melting curves of RT-qPCR products, supporting that only one species is amplified for each gene. 21C: plots showing that no template control does not amplify by RT-qPCR (C, values >32). 21D: a gel analysis of RT-qPCR product and shows that only one band (species) is generated.

FIGS. 22A-22Bplot a comparison of 5, 7, 9, and 10 for inhibition of pri-miR-27a biogenesis in MDA-MB-231 cells. 22A: the effect of 5, 7, 9, and 10 (negative control) on mature miR-27a levels, as measured by RT-qPCR. 22B: the effect of 5, 7, 9, and 10 (negative control) on pri-miR-27a levels, as measured by RT-qPCR. Error bars represent SD; n=3 biological replicates from 2 independent experiments. *, P<0.05; **, P<0.01; ***, P<0.001, as determined by a one-way ANOVA relative to 0 (untreated).

FIGS. 23A-23C provide plots showing that compound 9 inhibits miR-27a biogenesis in various cancer cell lines. 23A: the effect of 9 on mature and pri-miR-27a levels in MCF-7 cells, as determined by RT-qPCR. 23B: the effect of 9 on mature and pri-miR-27a levels in LNCaP cells, as determined by RT-qPCR. 23C: the effect of 9 on mature and pri-miR-27a levels in Hela cells, as determined by RT-qPCR. For all panels, error bars represent SD; n=3 replicates from 2 independent experiments. *, P<0.05; **, P<0.01; ***, P<0.001; P<0.0001, as determined by a one-way ANOVA relative to 0 (untreated cells).

FIGS. 24A-24B illustrate the specificity of 9 for pri-miR-27a vs. miRNAs in its cluster, pri-miR-23a and pri-miR-24. 24A: the secondary structure of miR-23a/27a/24 cluster wherein pri-miR-23a has SEQ ID NO: 255; pri-miR-27a has SEQ ID NO: 256; pri-miR-24-2 has SEQ ID NOs: 257, 295, 308, 309. 24B: the effect of 9 on mature miRNA levels in MDA-MB-231 TNBC cells, as determined by RT-qPCR (SEQ ID NO: 309). Error bars represent SD; n=3 replicates from 2 independent experiments. **, P<0.01, ***, P<0.001, as determined by a one-way ANOVA relative to 0 (untreated).

FIGS. 25A-25C plot the effect of 9 on protein expression in MDA-MB-231 cells, evaluated by global proteomics analysis. 25A: a volcano plot of the effect of 9 on the proteome of MDA-MB-231 cells, as determined by LC-MS/MS analysis, where the downstream targets of miR-27a are highlighted in dark grey. 25B: a volcano plot of the effect of 9 on the proteome of MDA-MB-231 cells, as determined by LC-MS/MS analysis, where the downstream targets of miR-23a are highlighted in dark grey. 25C: a volcano plot of the effect of 9 on the proteome of MDA-MB-231 cells, as determined by LC-MS/MS analysis, where the downstream targets of miR-24 are highlighted in dark grey. All downstream targets were predicted by TargetScanHuman v7.2. (4) Data in all plots are represented as Logz fold change; dotted lines represent a false discovery rate (FDR) of <5% and an S0 of 0.1 [where S0 is the minimum fold change required to be considered for significance], collectively an adjusted P-value of 0.05.

FIGS. 26A-26C shows Western blots and bar graphs showing treatment of MDA-MB-231 TNBC cells where 9 de-represses three downstream targets of miR-27a, PDCD4, PP4C, and ZBTB 10 26A: a representative Western blot to evaluate the effect of 9 on PDCD4 expression, relative to β-actin, in MDA-MB-231 cells and its corresponding quantification. 26B: a representative Western blot to evaluate the effect of 9 on PP4C expression, relative to β-actin, in MDA-MB-231 cells and its corresponding quantification. 26C: a representative Western blot to evaluate the effect of 9 on ZBTB10 expression, relative to β-actin, in MDA-MB-231 cells and its corresponding quantification. Error bars represent SD; n=2 replicates from independent experiments. *, P<0.05 as determined by a one-way ANOVA relative to 0 (untreated cells).

FIGS. 27A-27B demonstrate that compound 9 reduces the miR-27a-mediated migration of MDA-MB-231 TNBC cells. 27A: representative microscopic images of the effect of 9 at varying doses on the migration of MDA-MB-231, as compared to vehicle-, anti-miR-27a LNA-, and Scramble LNA (negative control)-treated cells. 27B: a plot showing quantification of the number of migratory cells, relative to vehicle-treated samples. Error bars represent SD; n=3 replicates from 2 independent experiments; 3 fields of view analyzed per sample. ***, P<0.001: ****, P<0.0001, as determined by a one-way ANOVA relative to “Vehicle”.

### Definitions

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

The term “and/or” in the context of this application means either one alone as well as both together, for example a substance including A and/or B means a substance including A alone, a substance including B alone and a substance including A and B together. Any one of the three choices standing alone may be made as well as any combination such as A alone as well as A and B together or B alone as well as A and B together or A alone, B alone and A and B together (e.g., all three choices).

It is also to be understood that as used herein and in the appended claims, the singular forms “a,” “an,” and “the” include plural reference unless the context clearly dictates otherwise, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder.

Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

At various places in the present specification substituents of compounds of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, isopropyl, n-butyl, sec-butyl, isobutyl, etc. For a number qualified by the term “about”, a variance of 2%, 5%, 10% or even 20% is within the ambit of the qualified number.

Standard abbreviations for chemical groups such as are well known in the art are used; e.g., Mc=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.”

The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66:1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts.

Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

Each of the terms “halogen,” “halide,” and “halo” refers to —F, —Cl, —Br, or —I.

The term “azide” or “azido” can be used interchangeably and refers to an —N₃ group (—N═N═N) which is bound to a carbon atom and is zwitterionic (carries a + and − charge respectively on the middle nitrogen and the terminal nitrogen). The azide group is a reactant in “click chemistry” which is a copper catalyzed azide-alkyne 1,3 dipolar cycloaddition (Sharpless etal., Angewandte Chemie, 41, 2596 et seq. (2002). A “hydroxyl” or “hydroxy” refers to an —OH group.

Compounds described herein include any small organic molecule including semi-synthetic, synthetic and/or naturally occurring organic compounds such as but not limited to aromatic, heteroaromatic, aliphatic, cyclic organic compounds, optionally substituted with functional groups such as but not limited to carboxyl, amido, ester, urethano, ureido, ether, sulfur, amino, hydroxyl, mercapto, sulfonyl, halogen, unsaturation and similar functional groups, combinations thereof and similar organic structures such as but not limited to terpenes, alkaloids, aromatic compounds, heteroaromatic compounds, nitrogen compounds, antibiotics, organic pharmaceuticals, sugars, peptides, nucleotides, and similar organic agents. Such compounds can exist in various isomeric forms, including configurational, geometric, and conformational isomers, including, for example, cis - or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound of the invention can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stercomerically pure compound having one chiral center will be substantially free of the opposite enantiomer of the compound. A stercomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

The terms “prevent,” “preventing,” and “prevention” refer to the prevention of the onset, recurrence, or spread of the disease in a patient resulting from the administration of a prophylactic or therapeutic agent.

A “patient” or “subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult.

The term miRNA means a microRNA sequence that is non-coding for peptides and functions at least for mRNA silencing and post-translational regulation of gene expression. Complementary base pairing of miRNA with messenger RNA molecules manages translation of the mRNA by up and/or down regulation, inhibition, repression and similar translation effects. Typical pre- and pri-miRNA sequences include structured and unstructured motifs. Groups of miRNAs often cooperate to manage mRNA function. An example is the pri-miRNA-17-92 cluster and the resulting pre-miRNAs and mature miRNAs produced by nuclease action on the cluster and pre-miRNAs respectively.

The terms pri-miRNA and pre-miRNA are the precursor RNA transcripts from which mature miRNA is produced. Transcription of DNA in the cell nucleus produces among other RNA molecules, pri-miRNA, a long RNA sequence which is capped and polyadenylated. Cleavage of the pri-miRNA and RNA chain processing in the nucleus produces the shorter pre-miRNA for export to the cellular cytoplasm. Pre-miRNA is further processed in the cytoplasm by RNase Dicer to produce double stranded short RNA and one of the two strands becomes mature, single strand miRNA for interaction with messenger RNA.

The term RNA motif refers to a targetable internal loop, hairpin loop, bulge, or other targetable nucleic acid structural motif, for example, as described in Batey et al., “Tertiary Motifs in RNA Structure and Folding.” Angew. Chem. Int. Ed., 38:2326-2343 (1999), which is hereby incorporated by reference. Examples of RNA motifs include symmetric internal loops, asymmetric internal loops, 1×1 internal loops, 1×2 internal loops, 1×3 internal loops, 2×2 internal loops, 2×3 internal loops, 2×4 internal loops, 3×3 internal loops, 3×4 internal loops, 4×4 internal loops 4×5 internal loops, 5×5 internal loops, 1base bulges, 2 base bulges, 3 base bulges, 4 base bulges, 5 base bulges, 4 base hairpin loops 5 base hairpin loops, 6 base hairpin loops, 7 base hairpin loops, 8 base hairpin loops, 9 base hairpin loops, 10 base hairpin loops, multibranch loops, pseudoknots, and the like. RNA motifs have known structures. A structured motif of any kind of RNA is a segment of the RNA having a stable three-dimensional structure that is not wholly dependent upon the particular nucleotide sequence of the structure motif. Hairpins, bulges, terminal (internal) loops, multibranch loops, and pseudoknots formed by RNAs are typical structured motifs. RNAs such as but not limited to pri-miRNA, pre-miRNA, tRNA, rRNA, long non-coding RNAs, mRNAs, particularly untranslated regions, viral RNAs, bacterial RNAs, and others typically have structured motifs. A synthesized RNA construct is any kind of RNA prepared by a synthetic technique and typically will be at least a structured motif of any kind of RNA.

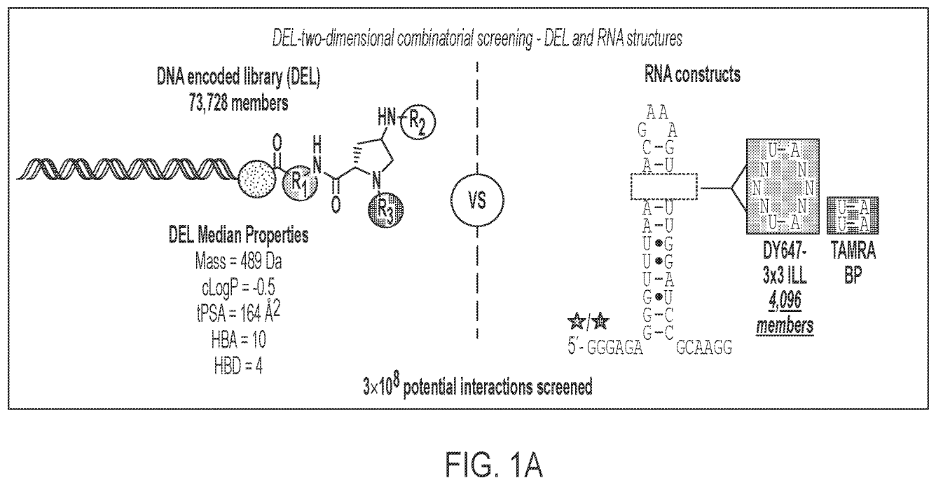

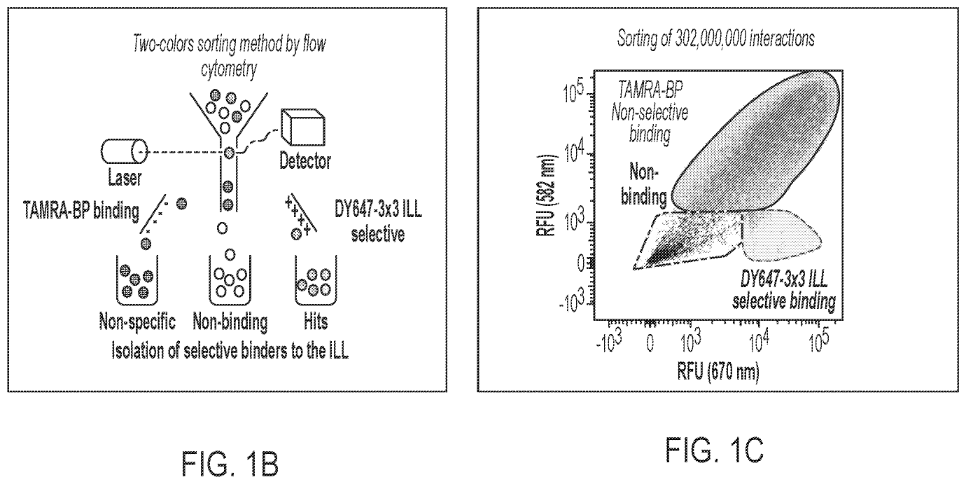

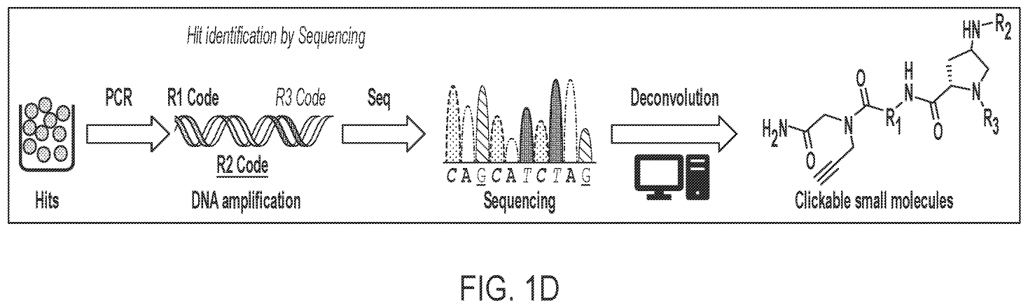

The term 3×3 ILL synthetic RNA construct is synthesized RNA having a 3×3 nucleotide internal loop that can be constructed of a random arrangement of the four RNA phosphorylated nucleotides AUGC so as to provide up to 46 combinations or up to about 4,096 combinations. The 3×3 ILL synthetic RNA construct is described in US 2016/188791 and depicted in FIG. 1 of this '791 publication.

### DETAILED DESCRIPTION OF THE INVENTION

Mathematical solution of two body or variable problems has always been a challenge. Fourier and LaPlace transforms often are applied to provide a series of solutions for these problems. A similar issue arises in the course of determination of structure-activity relationships for biological research when the biological target and the medicinal agent are both variable. Typical research of this kind fixes one of these variables and uses the fixed factor to search for hits among libraries of the other variable. This solution, however, lacks elegance and precise targeting. Hits from among the variable library often result in further, difficult structural synthetic variation to develop a suitable result. The variable library is also an issue because it is typically chosen for its pre-existing relevance to the biological target. The pre-choice eliminates significant structural diversity for hits from the variable library.

A solution for this two body problem in the context of libraries of RNA sequences and compounds including semi-synthetic, synthetic and/or naturally occurring organic compounds, also synonymously designated herein as small molecules, comprises the combination of two screening techniques, the DNA encoded small molecule library technique (DEL) and the two dimensional combinatorial screening technique (2DCS). The DEL technique enables screening of a small molecule library by tagging each small molecule and/or its immobilization site with a unique DNA tag. Reading the tag provides the identity of the small molecule. The 2DCS technique probes RNA and chemical spaces simultaneously.

The combination of these two techniques enables solution of the two body problem. Use of the DEL technique with a tagged small molecule library and a multitude of synthetic RNA sequences displaying all possible nucleotide variations for a discrete structural motif (a 3×3 internal loop or a 6-nucleotide hairpin, for examples) provides small molecule hits targeting the structured motif. Subsequent use of the small molecule hits with a library of RNA sequences displaying ordered variations of the same structured RNA motif identifies small molecules that selectively bind with certain identifiable RNA sequences.

In particular, the DEL method for screening compounds by DNA encoded labeling. comprises the steps of first forming an immobilized compound library of small molecular compounds having DNA labels. Although the present invention is applicable to all kinds of semi-synthetic, synthetic and/or naturally occurring organic compounds, the exemplary compounds used herein are peptides synthesized from natural and synthetic amino acids through use of the Merrifield synthesis technique (See Wikipedia at “en.wikipedia.org/wiki/Peptide_synthesis”). Each microsupport also carries a unique DNA label of from 6 to 12 nucleotides and a PCR amplification sequence. This step of the DEL method is illustrated by the left side of FIG. 1-A.

In the next step of this DEL method, the immobilized compound library is combined with two RNA samples. The first sample is an RNA library comprising synthesized RNA constructs with fluorescent labels wherein the synthesized RNA library construct displays a randomized region of their 3×3 nucleotide internal loop pattern (this RNA library is illustrated by the right side of FIG. 1A). The second sample is an RNA counter construct comprising a fully base paired RNA; that is, the randomized region of their 3×3 nucleotide internal loop pattern is replaced with base paired nucleotides. The fully base-paired RNA is labeled with tetramethylrhodamine to identify and rule out non-selective RNA-binding compounds from the screen.

The compound-RNA binding results produce:
a) a first subgroup of the immobilized compound library bound to the RNA library.
b) a second subgroup of the immobilized compound library bound to the non-selective RNA counter construct, and
c) a third unbound subgroup.

These groups are sorted by flow cytometry using fluorescent detection sorting and isolating the first, second and third subgroups by flow cytometry using fluorescent detection. This sorting method is illustrated by FIG. 1B.

Amplifying the DNA labels of each of compounds of the first subgroup and reading (sequencing) the amplified DNA labels enables identification of each of the compounds of the first subgroup. Compounds of interest are identified as being present as replicate hits in the first subgroup and absent in the other subgroups. A replicate hit is defined as a compound that is described by multiple different DNA sequences that collectively suggest that multiple microsupports displaying the same compound were sorted. This step is illustrated by FIG. 1D.

The 2DCS technique is then used to identify RNA sequences that selectively bind with the small molecular compounds scored as hits by the DEL technique. The DEL compounds scored as hits are immobilized with click chemistry onto an azide-functionalized microarray to produce a microarray of conjugated small molecular compounds. The click chemistry immobilization functions through an acetylene-azide copper catalyzed formation of a triazole. Each of the small molecular compounds is bound to an individual solid microsupport through use of the compound anchor, a propargyl group which forms a triazole ring with azide on the microsupport.

The microarray of conjugated compounds is then combined with the RNA library in the presence of competitor oligonucleotides, according to the procedures of the 2DCS technique: a second radiolabeled RNA library described above except the library is radiolabeled rather than fluorescently labeled and a panel of competitor oligonucleotides. This second, radioactively labeled, RNA library comprises the synthesized RNA constructs of the first library from the DEL technique but without the fluorescent labels. Instead, the RNA constructs of this third library are labeled with radioactive phosphorus labels. The competitor panel comprises nucleotide sequences that mimic regions common to all members of the RNA library as well as fully paired RNA and DNA oligonucleotides (AU, AT, or GC base pairs). The combination of the conjugated compounds, the RNA library, and the panel of competitor oligonucleotides produces a microarray of at least some conjugated compounds bound to certain members of the second RNA library and at least some conjugated compounds bound to certain competitor oligonucleotides, which are silent in the selection as they are unlabeled.

The microarray can be washed to remove unbound synthesized RNA constructs and unbound oligonucleotides. The microarray is then harvested to obtain the bound synthesized RNA constructs of the microarray wells displaying radioactivity. The harvested RNA constructs are sequenced and read to produce a sequence data set. The sequence data set is statistically analyzed to determine if the enrichment of a selected RNA in the RNA-seq analysis is statistically significant, by comparison the RNA library not subjected to the selection process. These statistically significant interactions are true binding events that define the small molecule's affinity landscape.

The statistical significance of enrichment in the 2DCS selection affords a hierarchy of bound RNAs, with those that are most statistically significant being highest affinity. The high statistical ratings of selected RNA sequences are folded in silico to determine their structures. These structures are compared to natural RNAs to determine if they house a targetable structure, that is that they will selectively and significantly bind with a small molecular compound of the first subgroup from the DEL technique.

By application of the combination of the DEL and 2DCS techniques, the peptide compounds of Formula I, and specifically the peptide compounds of Formulas pc1, pc2, pc3, pc4 and pc5 were identified. Details of application of the DEL and 2DSC techniques and biological assay and examination of the peptide compounds illustrates the power of this technique.

### Design of the DNA-Encoded Small Molecule Library and the RNA 3D Fold Library

The DEL was synthesized and validated following published protocols. (15, 24) Each bead contained sites for compound synthesis and sites for enzymatic ligation of the encoding oligonucleotide. During encoded combinatorial synthesis, each chemical building block was assigned a unique short DNA sequence that was ligated to the beads after coupling the building block. The PCR primer binding sites were installed prior to initiating and after completing encoded combinatorial synthesis, yielding DEL beads that display PCR-amplifiable DNA encoding tags (Scheme 1, Tables 1 & 2).

The physical properties of RNA-binding antibacterial natural products, some of which exhibit unconventional physiochemical properties, inspired the DEL design and building block pool composition. Each library member contains one of 96 amino acids (R1), a central Fmoc-Pro (N₃)—OH hub, then one of 192 carboxylic acids. The carboxylic acid was installed after either Fmoc deprotection and acylation of the pendant secondary amine (R2) or Staudinger reduction of the azide and acylation of the pendant primary amine (R3). The remaining pendant Fmoc groups were removed, and azides reduced to yield a 73,728-member DEL of diverse primary or secondary amine-containing compounds. A stoichiometric mixture of two Fmoc-Pro (N₃)—OH diastereomers was used for the central hub coupling, and this position was not encoded. Amino acid and carboxylic acid building blocks were selected that display diverse tertiary amine and hydroxyl functionalities. Thus, DEL members tended to be higher in molecular weight, more hydrophilic, and more functionalized with hydrogen bond donors and acceptors compared to chemical matter contained in previous solid phase DEL screening studies (Table 3, FIGS. 1-A, S1, & S2). (25, 26)

The DEL was screened using a previously described RNA fold library. (22, 27) The RNA structure library displays a randomized region in a 3×3 nucleotide internal loop pattern (4,096 members) fluorescently labeled with DY647 (DY647-3×3 ILL) (FIG. 1A). The randomized region is embedded into a unimolecular hairpin with single-stranded 5′ and 3′ tails that contain primer binding sites for PCR amplification and sequencing. (27) As a counter screen to eliminate molecules that bind RNA non-selectively, we used a tetramethylrhodamine-labeled RNA in which the 3×3 nucleotide randomized region was replaced with base-paired nucleotides (TAMRA-BP; FIG. 1A).

### FACS Analysis of Solid-Phase DEL RNA Structure Library Binding Assay

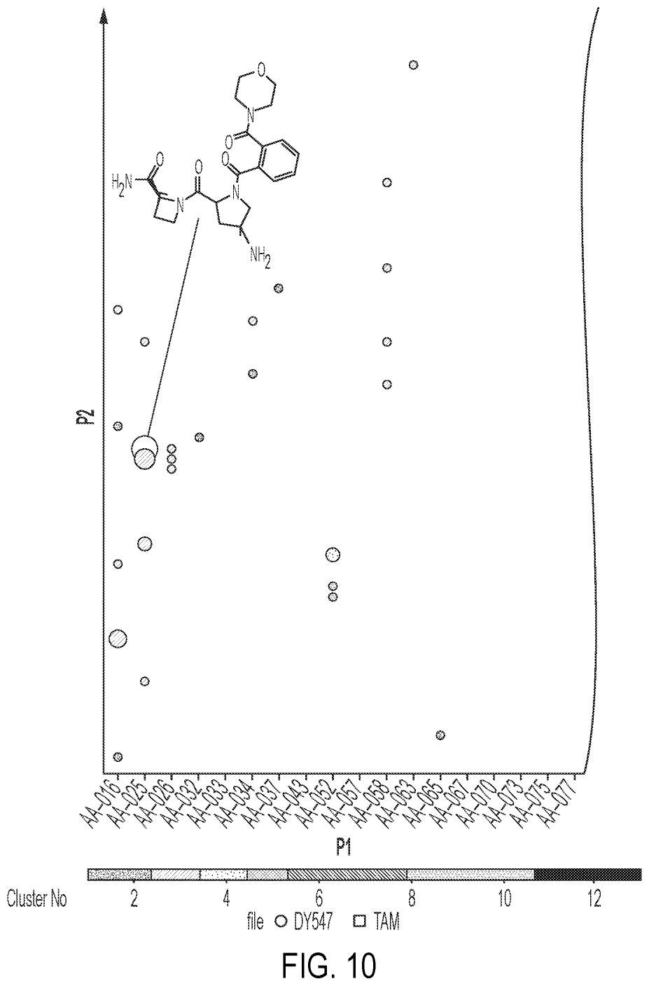

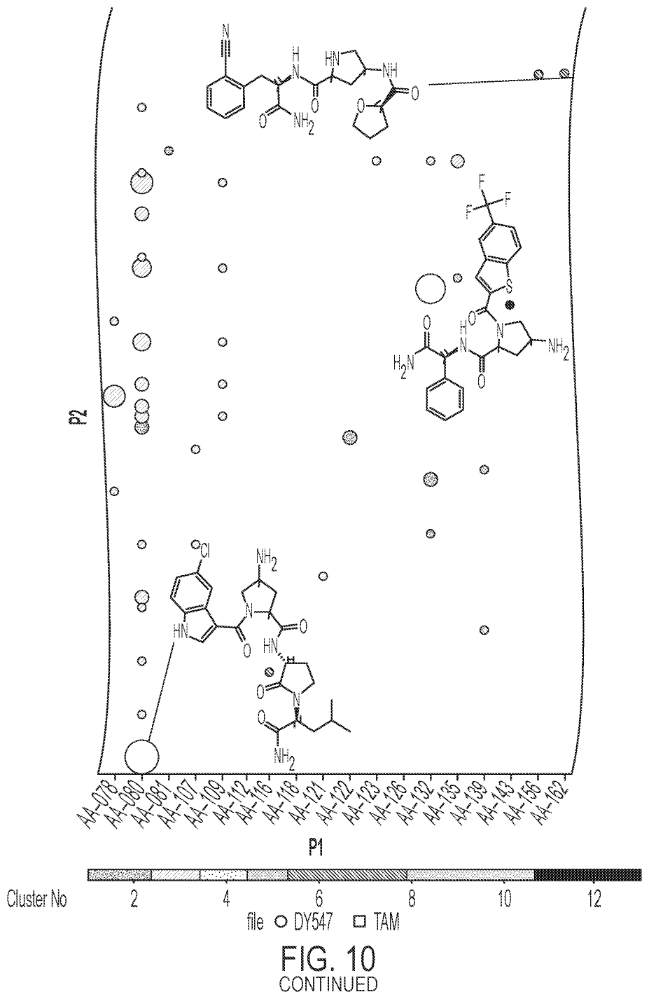

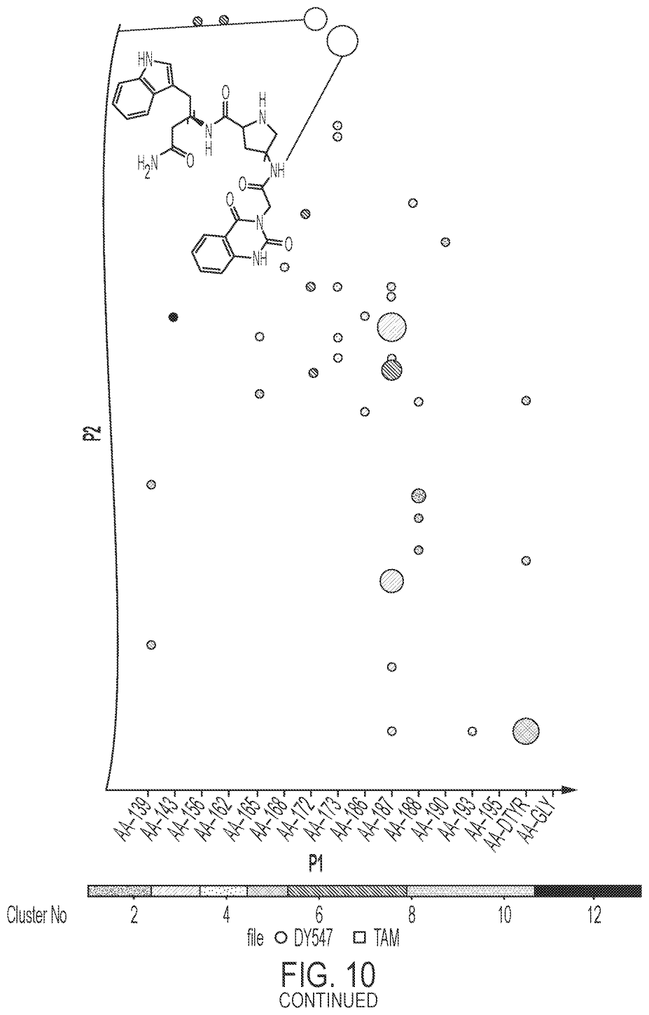

An aliquot of the DEL (˜750,000 beads) was incubated with DY647-3×3 ILL and TAMRA-BP, bovine serum albumin (BSA), and tRNA. Two-color FACS analysis isolated DEL beads that bound specifically to DY647-3×3 ILL by sorting beads with high DY647 fluorescence (λₑₓ/λₑₘ=652/673 nm) and low TAMRA fluorescence (λₑₓ/λₑₘ=555/580 nm) (FIGS. 1-B, 1-C & S3). The analysis yielded 102 DY647 sort events (0.01% hit rate) and 60 TAMRA sort events (0.008% hit rate); most RNA-binding beads bound both DY647- and TAMRA-labeled RNA, i.e., non-selectively (FIG. 10).

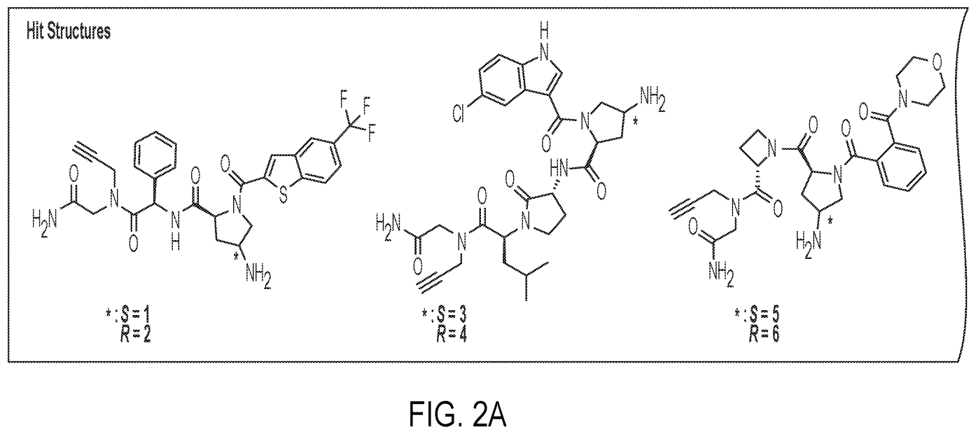

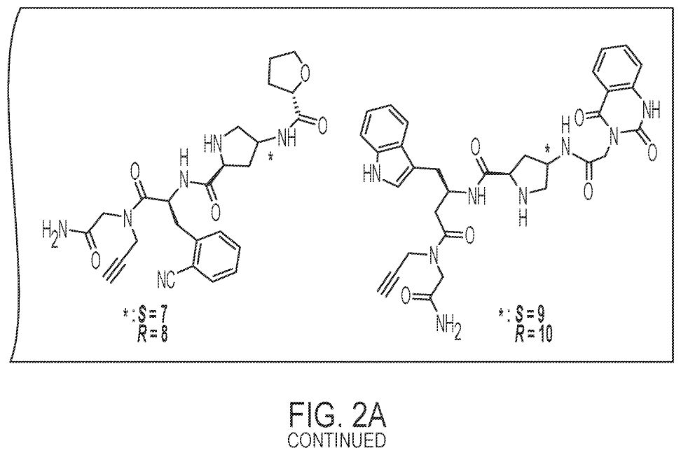

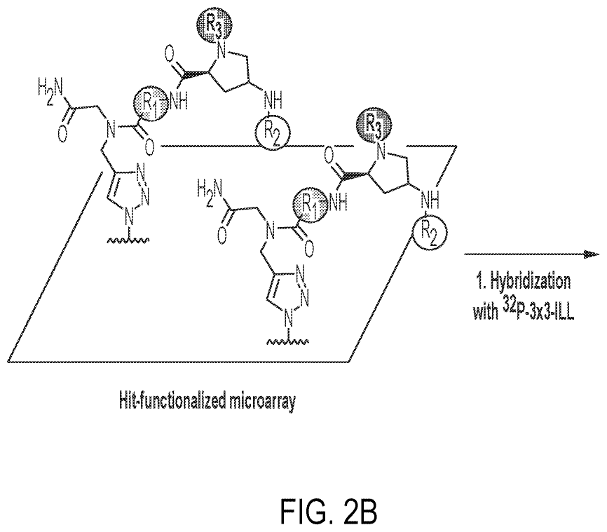

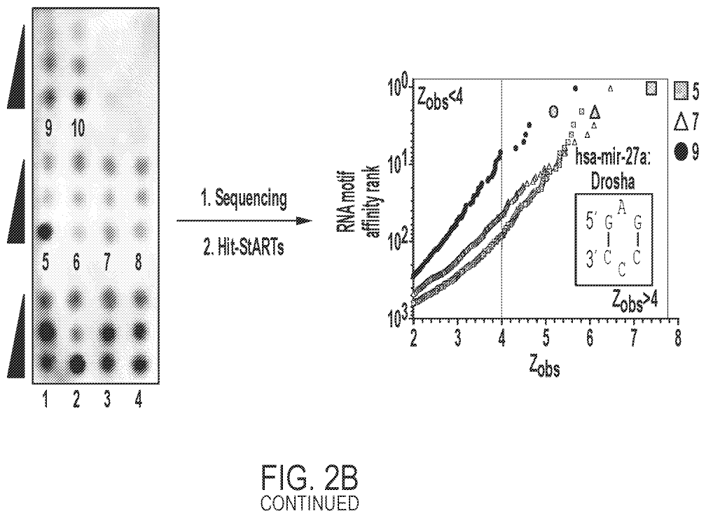

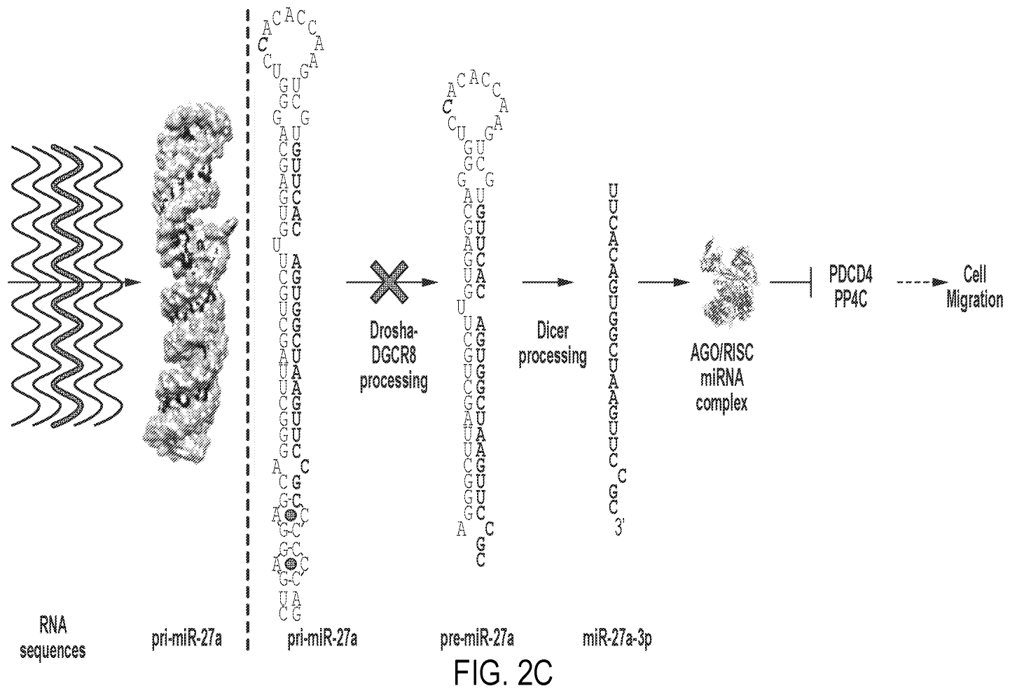

DEL bead hits were pooled, amplified, sequenced, and decoded to identify hit structures for structure-activity analysis and for prioritization for synthesis and subsequent validation. Sequence pattern matching identified unique DY647 and TAMRA hit beads, (25) and hit structures were clustered according to Tanimoto chemical similarity (0.8). (28) Hits were then ranked in each cluster according to their replicate (k) class, that is, the number of times the same compound was observed as a hit on different FACS-sorted beads (FIGS. 1-D & S5). (24, 29) Each library member was screened as a mixture of two Pro (N₃) diastercomers, thus each hit was synthesized as optically pure compounds in diastereomer pairs (e.g., compounds 1 and 2). Hits 1-10 (pc1(S), pc2(R), pc3(S), pc4(R) pc (5S), pc6(R) pc7(S), pc8(R) pc9(S), pc10(R), hereinafter 1-10) are the highest k class representatives of 5 of the top 6 most populous clusters of DY647 hits. Hits 5/6 (k=6) represent the largest hit cluster (16 additional k>1 hit structures). Hits 3/4 (k=9), 7/8 (k=5), and 9/10 (k=8) were also high k class hits and were the representative of small (1- or 2-member) clusters (FIG. 2A).

Several common features of hit structures appeared to be important for selectively binding the RNA structure library, guiding our selection of hits for scaled synthesis and further exploration. The top three represented cycle 1 building blocks were the Freidinger's lactam of hits 3/4, D-threonine, and the azctidine of hits 5/6 (40, 21, and 14 hits of the 181 DY647 hits, respectively). The top represented cycle 2 building blocks included the chloroindole of hits 3/4, the morpholine-carbonyl benzoic acid of hits 5/6, the trifluoromethyl benzothiophene of hits 1/2, the tetrahydrofuran of hits 7/8, and the dioxo-dihydroquinazoline of hits 9/10. The dioxo-dihydroquinazoline representation resulted from a single high k class hit structure, indicating that this structure was important for RNA binding and likely required the specific cycle 1 L-B-homotryptophan context. Overrepresentation in cycle 2 is notable as this is the highest diversity cycle (192 acids). Acylation at the Ca N via Fmoc deprotection was preferred to acylation at the Cy N via azide reduction for ILL-selective ligands (137 vs. 44 DY647 hit beads, respectively). Thus, hit structures collectively explored overrepresented building blocks and acylation position in the context of high k class hit structures.

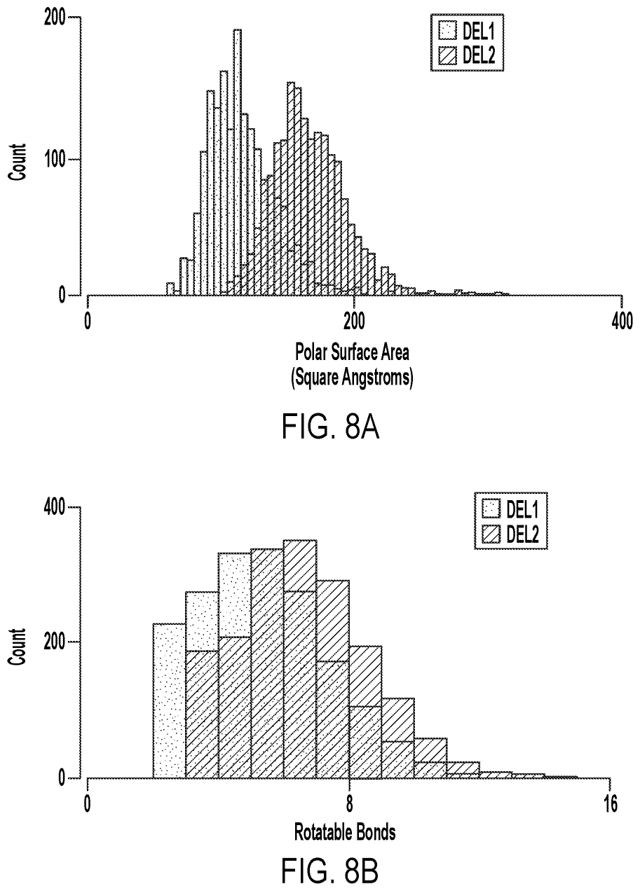

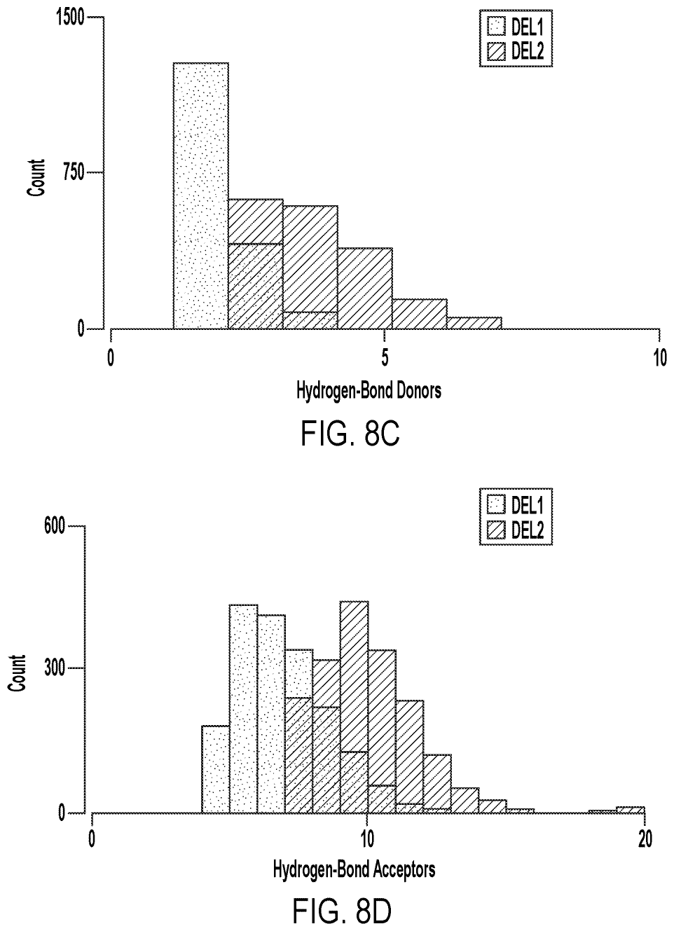

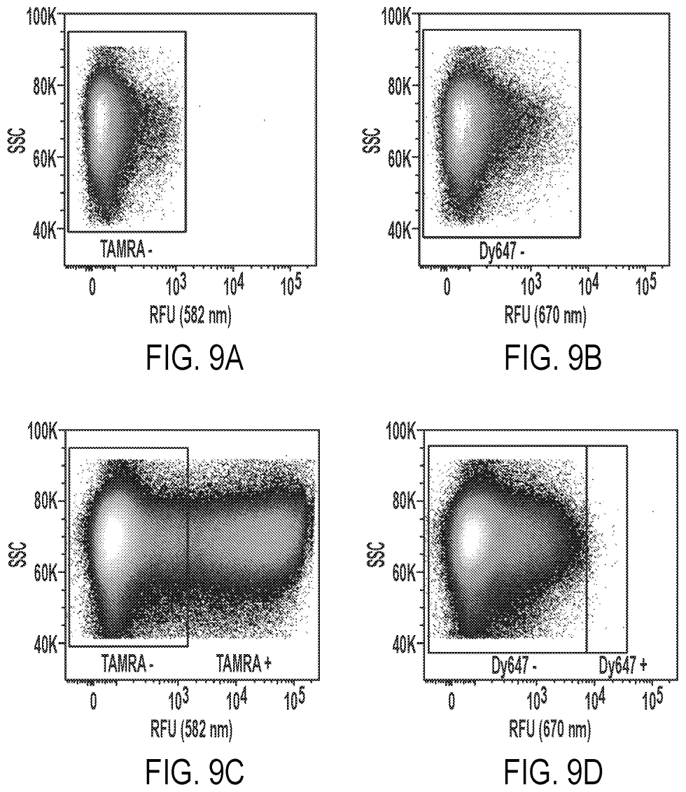

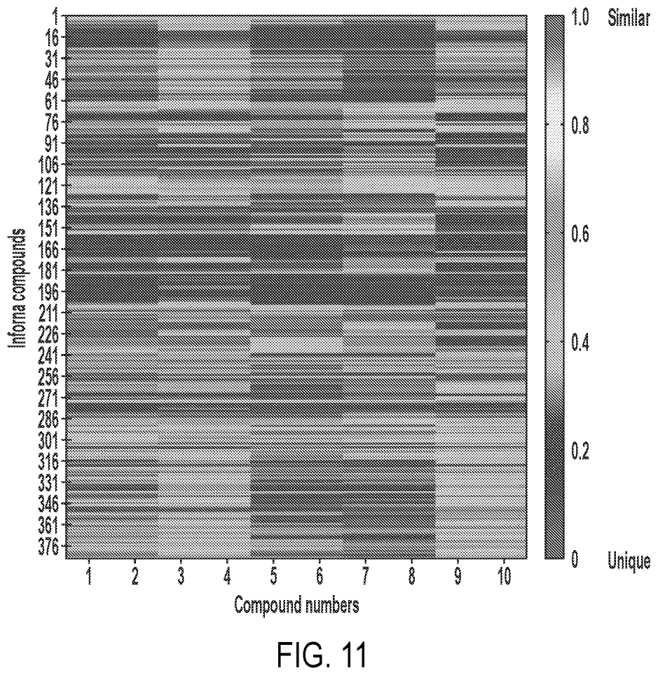

Owing to the DEL design, the physicochemical properties of compounds 1-10 are similar to known RNA-binding small molecules. LogD and LogP tend to be lower and the number of hydrogen bond donors and acceptors tend to be higher compared to DELs that were designed and synthesized to sample canonical Lipinski-Veber drug-like chemical space (FIGS. 8A-8D & Table 4). (2, 3, 25) Despite these similarities, however, 1-10 are chemically dissimilar to known RNA-binding compounds. The average Tanimoto score for each compound against all small molecules housed in Inforna, an expansive database of known ligand-RNA interactions, is 0.3±0.01 (FIG. 11). Therefore, the DEL screen identified new chemotypes that bind RNA 3D folds.

Identification of the RNA 3D Folds that Bind to SPDEL Compounds Via 2DCS

The interaction landscape for the ten representative DEL compounds emerging from the FACS screen were then defined by 2DCS (22). In 2DCS, small molecules are site-specifically conjugated to a microarray surface and then probed for binding to an RNA library under highly stringent conditions (FIGS. 2-B & S6). That is, the array is co-incubated with a radioactively labeled RNA library and an excess of competitor oligonucleotides that mimic regions common to all library members. (30) as compared both to the total number of moles of compound delivered to the array surface and the RNA library. To enable site-specific immobilization of compounds 1-10, a C-terminal alkyne handle was installed for CuAAC click (30) coupling to azide-displaying array surfaces. Installation of the alkyne was accomplished by coupling of bromoacetic acid onto rink amide resin followed by nucleophilic displacement with propargylamine prior to the synthesis of the SPDEL compounds (FIG. 2A).

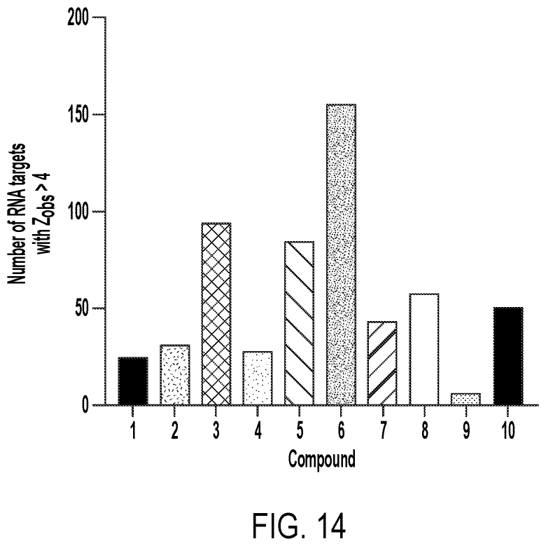

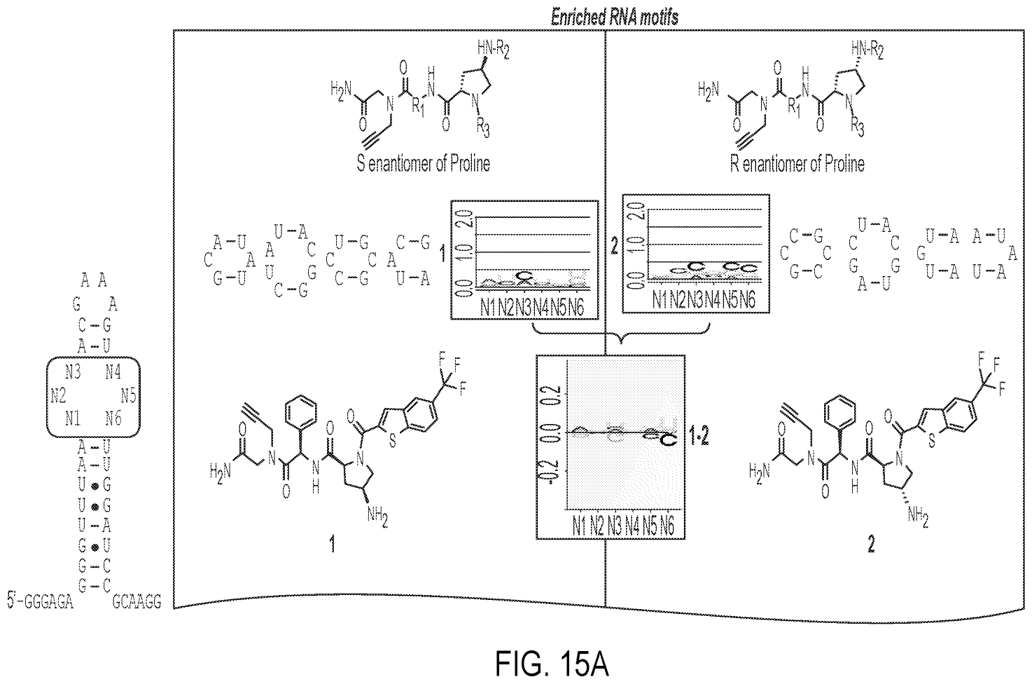

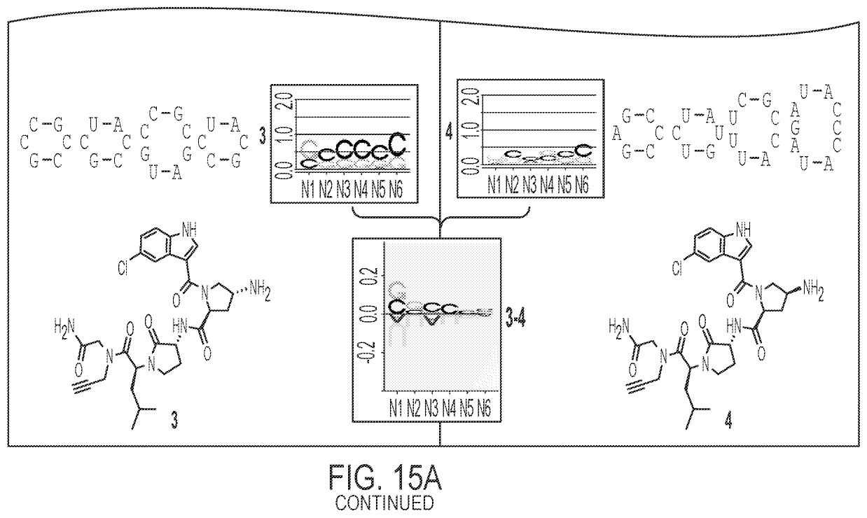

All ten compounds bound to the 3×3 ILL under the highly stringent conditions described above, almost all dose-dependently (FIG. 2B). The RNAs bound to each small molecule were harvested, reverse transcribed into DNA, and sequenced by RNA-seq. The resulting data set was statistically analyzed by using High Throughput Structure-Activity Relationships Through Sequencing (Hit-StARTS) (31) in which the relative frequency of each selected RNA is compared to the frequency of that RNA in the starting library (pooled population comparison). (31) The statistical significance of the enrichment of selected RNAs is quantified by Z_{obs}, which correlates with affinity and selectivity. (31) Here, a cut-off of Z_{obs} >4 was employed, which was empirically determined from measured affinities via evaluation of compound 9 binding with potential RNA motifs extracted from the 2DCS (FIGS. 2-B, 13, & 14). Notably the direct correlation between Z_{obs} and binding affinities was demonstrated (FIG. 14). This cut-off afforded, on average, 60±40 (range: 6-155) preferred, or privileged, RNA 3D folds for each compound. Compound 6 bound to largest number of RNA targets (n=155) while compound 9 bound the fewest (n=6) (FIG. 14). Interestingly, each compound bound a set of RNAs that is unique, ranging from 2 (compounds 4 and 9) to 41 motifs (compound 3). Altogether, 1-10 bound 212 unique 3D folds of which 31 motifs (14%) previously had no known ligand binding partner. Of the 212 new motifs, none is present in human transfer RNA (tRNAs), and only 24 are present in human ribosomal RNA (rRNA). (32)

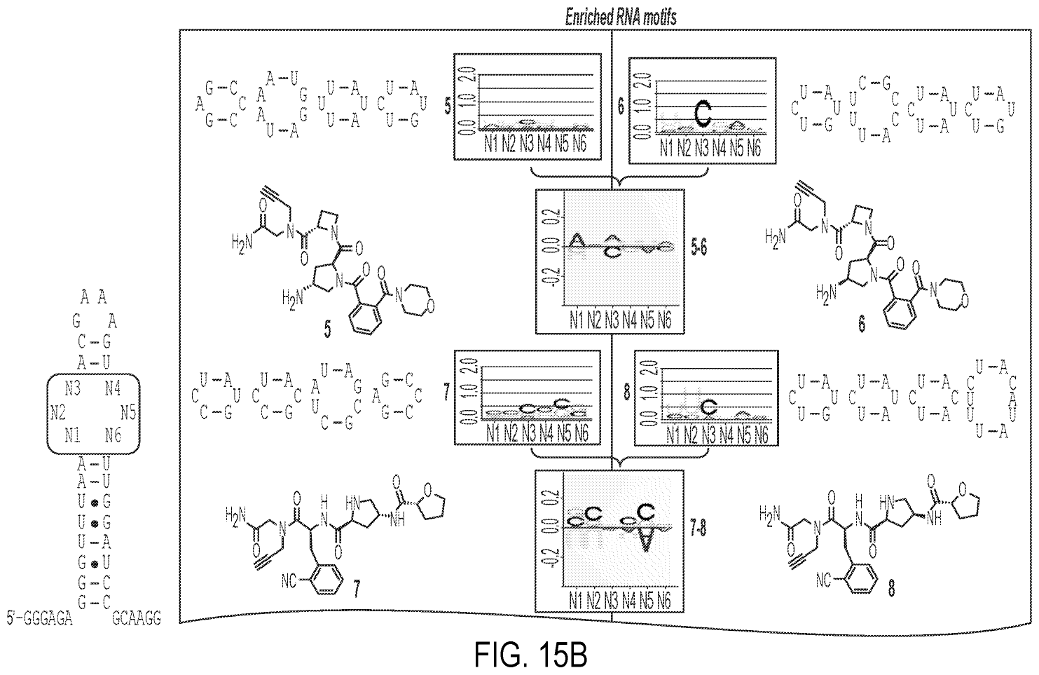

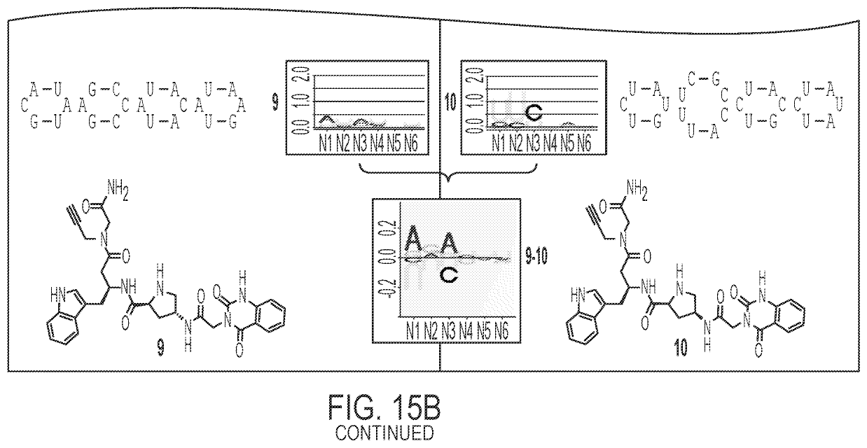

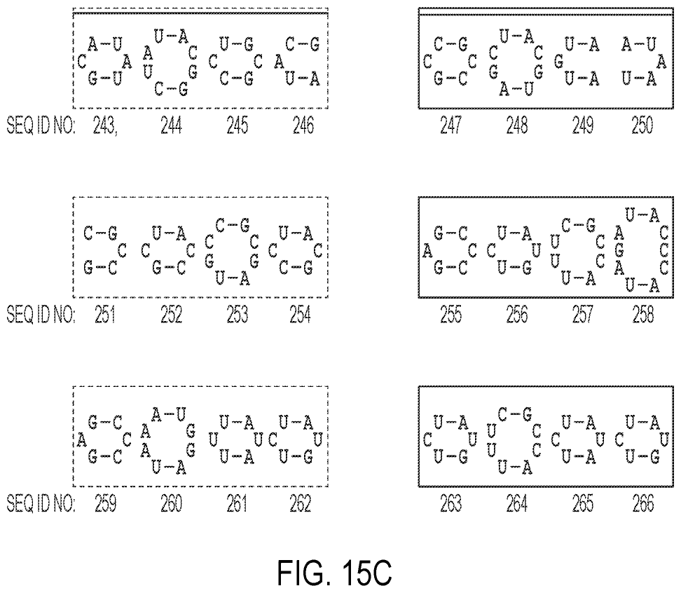

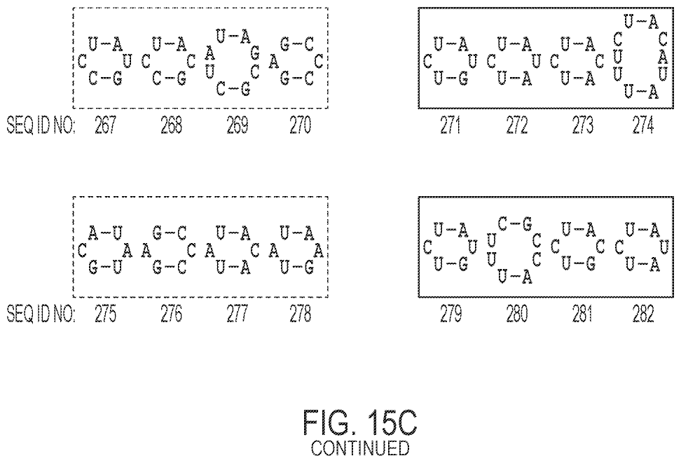

LOGOS, which graphically depicts sequence preferences as “bits” of information, were generated for each DEL-derived ligand (FIG. 15). (33) DiffLOGO (34) revealed that some diastereomers of the same compound (1 and 2; 3 and 4, etc.; odd numbers are the S diastereomer, even numbers are the R diastereomer) are partial to similar sequences while others are very different (FIG. 15). For example, 9 prefers internal loops with A's while 10 selects pyrimidine-rich loops. In contrast, the four loops with the highest Z_{obs} scores for 1 and 2 are very similar, and 3 and 4 both selected pyrimidine-rich loops, particularly cytosines (FIG. 14). Interestingly, three of the R diastercomers, 6, 8, and 10, have similar LOGOS with strong preferences for U in positions 1 and 2 and C in position 3. Further, these trends are not observed in their R diastereomers (which generally have less sequence preference throughout all positions of the randomized region), suggesting that the proline stereochemistry influences its preferred RNA 3D folds (FIG. 15). Thus, these data suggest the stereochemistry of a ligand influences its molecular recognition fingerprints for RNA targets.

### SAR on the Human Transcriptome

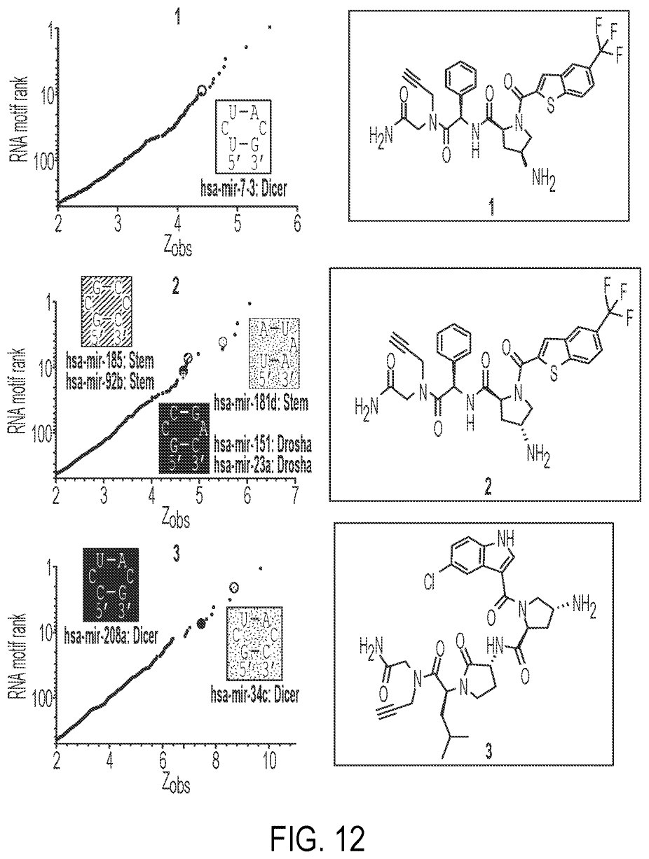

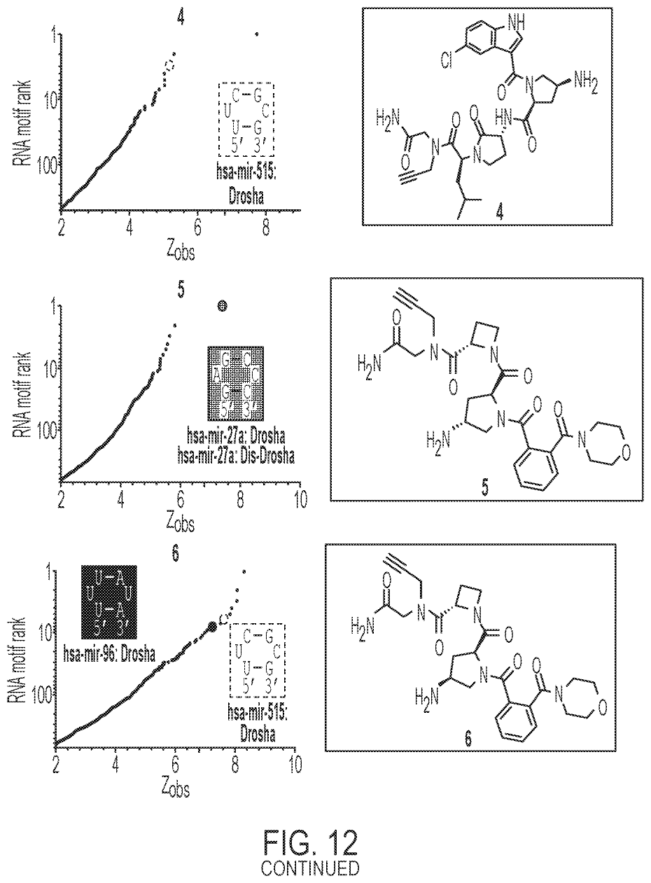

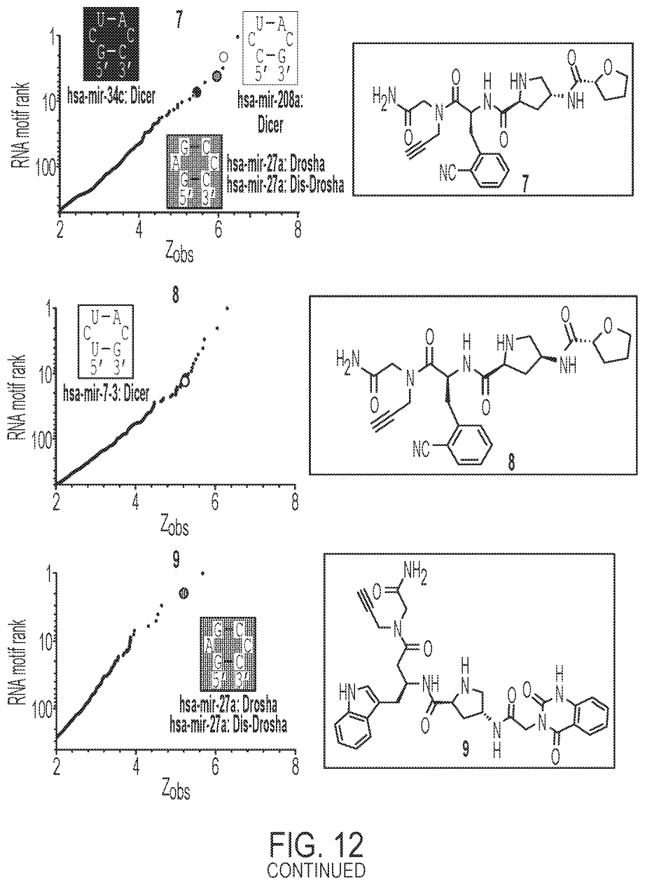

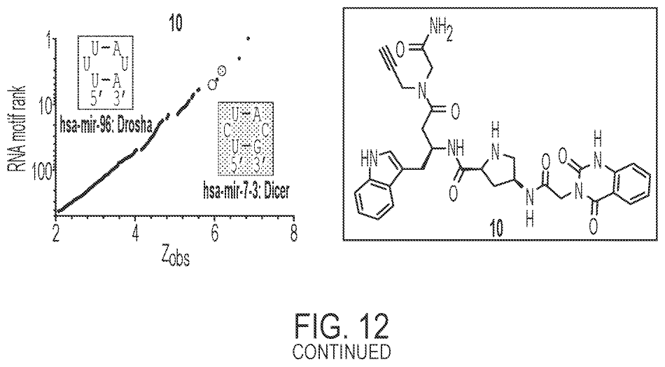

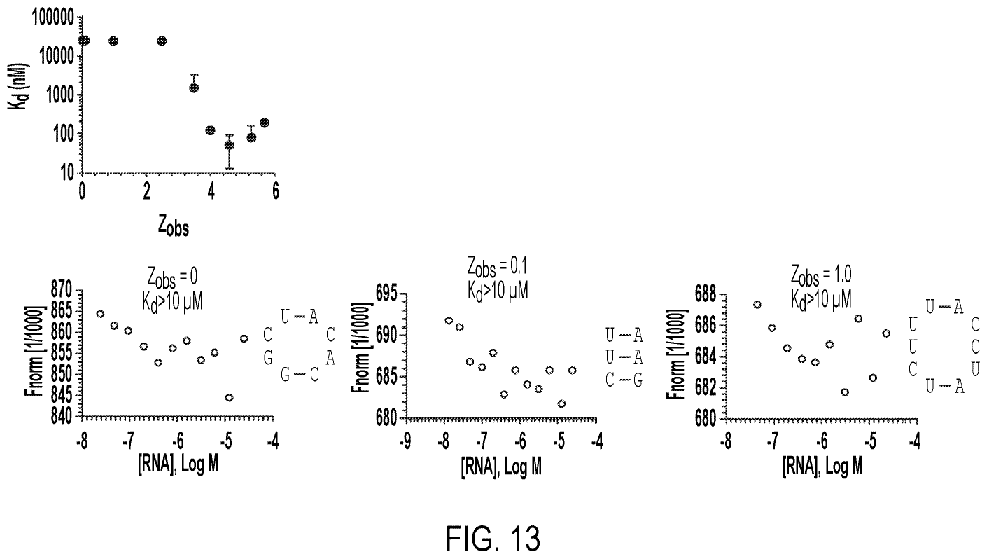

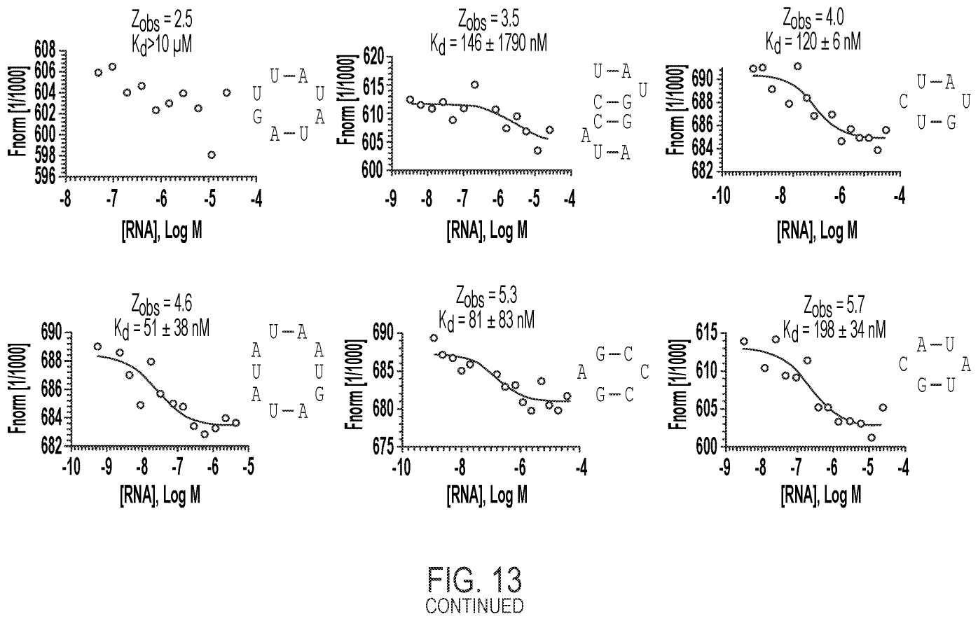

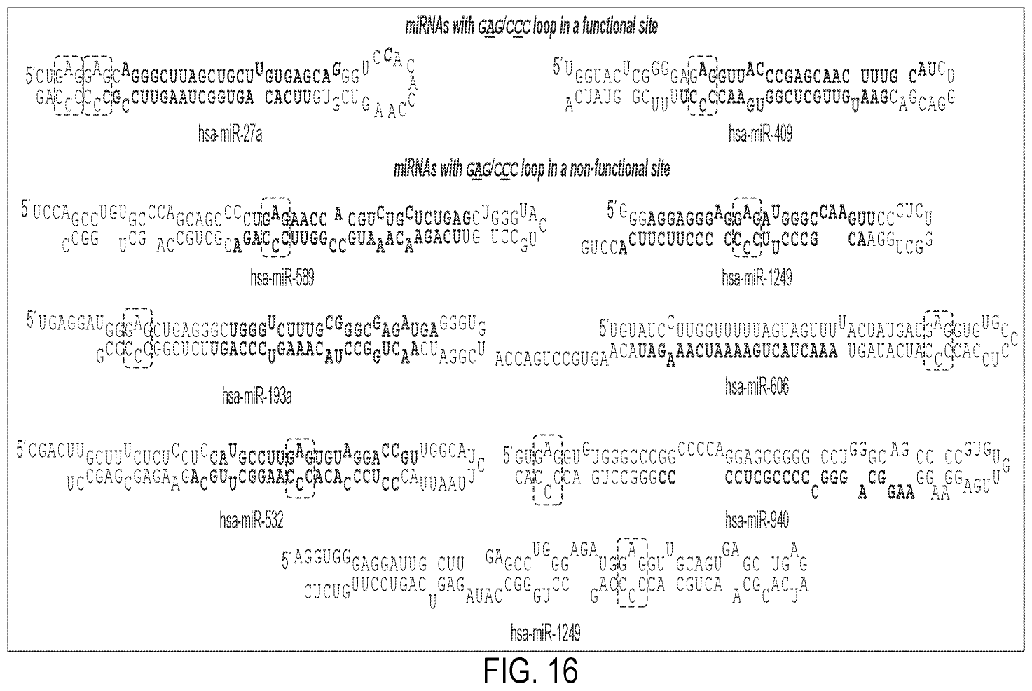

Inforna provides unprecedented ability to define SAR on the human transcriptome. A compound's selectivity is a function of the number and type of RNA folds that it binds, the frequency of the folds in the transcriptome, and the expression level of the RNAs in which they reside. Therefore the human miRNome in miRBase (35) was mined for the 212 new RNA motifs with Z_{obs}>4.0 that bound 1-10. Of these motifs, 123 (58%) were present in human miRNome, and eight were in Drosha or Dicer processing sites of a disease-associated miRNA (FIG. 12). Interestingly, three compounds, 5, 7, and 9, were predicted to bind to the 5′GAG/3′CCC internal loop present in the Drosha processing site of pri-miR-27a, albeit with different avidities based on their Z_{obs} scores; miR-27a is an oncogenic miRNA with established roles in breast and prostate cancers. (36-38) In addition to the 5′GAG/3′CCC loop present in pri-miR-27a's Drosha site, a second copy of the loop is also present nearby (FIG. 16). Binding of its Drosha site with a ligand might inhibit miR-27a biogenesis and ablate oncogenic phenotypes in cancer cell lines (FIG. 2C). Although the 5′GAG/3′CCC motif of interest is present in nine human miRNAs, it is only present in processing sites of miR-27a and miR-409 (Drosha) and, as such, would only be predicted to affect the processing of these targets (FIG. 16). Of the two, only miR-27a is significantly expressed across various cell lines/types, as indicated by the number of reads per million from the sequencing data available in the miRBase 22.1 (October 2018); (35) miR-27a is expressed at ˜220-fold greater levels on average than miR-409 (for miR-27a: 85,900 reads per million, 3554414 total reads analyzed over 159 experiments; for miR-409a: 391 reads per million, 19718 reads analyzed over 128 experiments). (35) Therefore, further studies on the small molecule-targeting of pri-miR-27a with 5, 7, and 9 were undertaken.

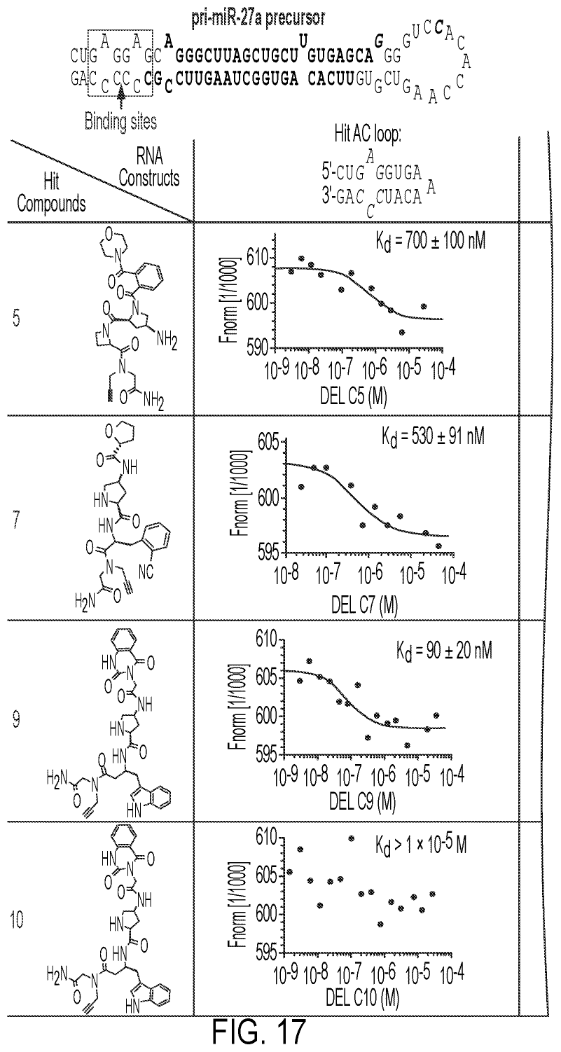

First, the in vitro binding affinity of these compounds towards the putative preferred binding motif, 5′GAG/3′CCC, by microscale thermophoresis was measured (FIG. 17). Compound 9 demonstrated the highest binding affinity with K_{d} of 90±20 nM, followed by 5 and 7 with K_{d}s of 700±100 nM and 530±90 nM, respectively. To study the potential importance of the closing base pairs, the affinity landscape was mined for related internal loops that did not bind the small molecules. None of the molecules were predicted to bind an AC internal loop in which the 5′ closing base pair was mutated from GC to CG (indicated in bold), that is 5′CAG/3′GCC. Interestingly, this loop is adjacent to the 5′GAG/3′CCC that binds 5, 7, and 9 in pri-miR-27a (FIG. 2C). Saturable binding was not observed for any of the three compounds for the mutated loop (K_{d} >50 μM) or for a fully paired RNA (FIG. 17). Moreover, 10, a stereoisomer of 9, binds to none of the three RNAs, indicating the crucial role of 9's stercochemistry for binding (FIG. 17).

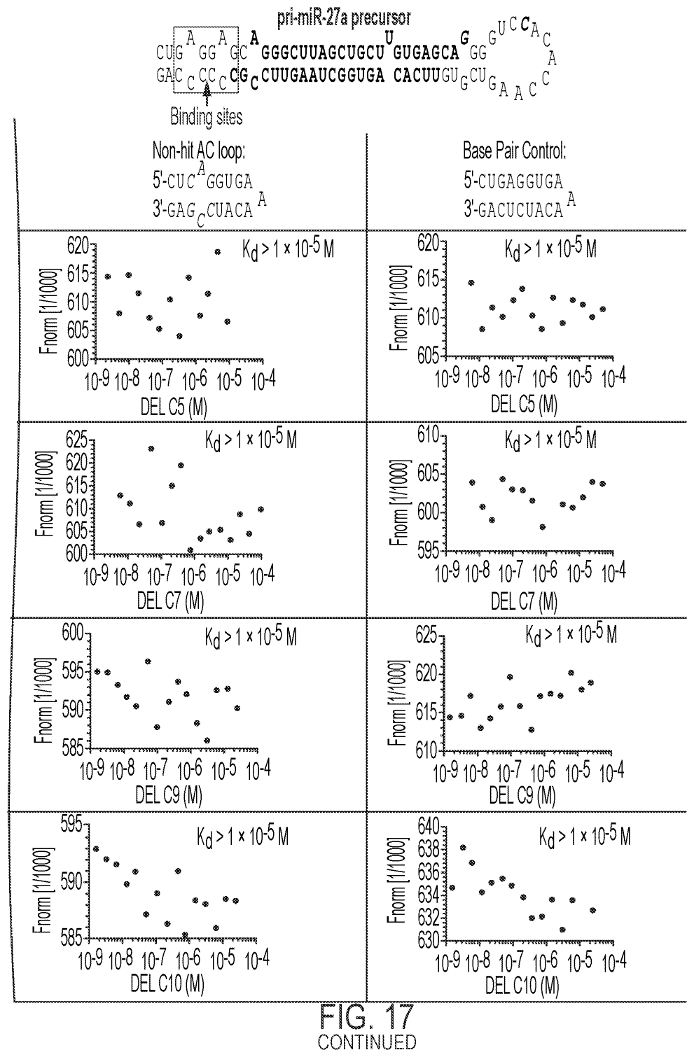

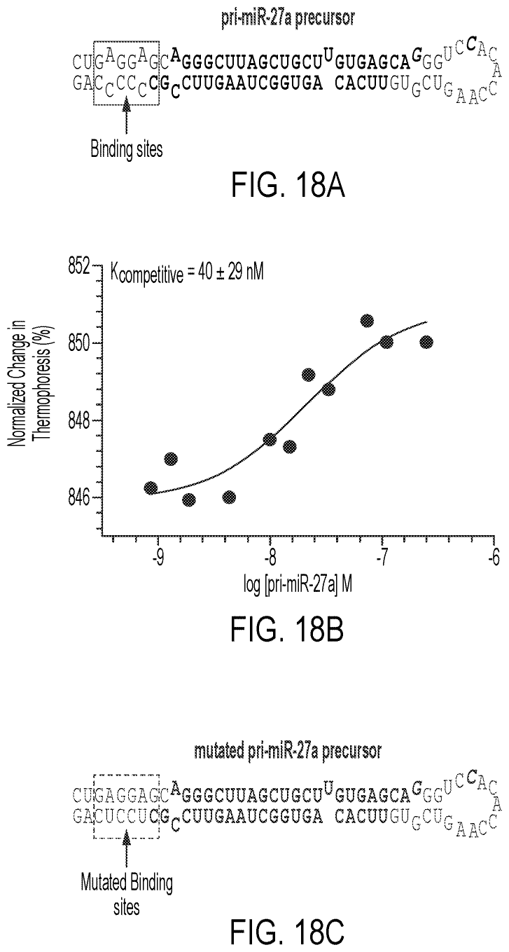

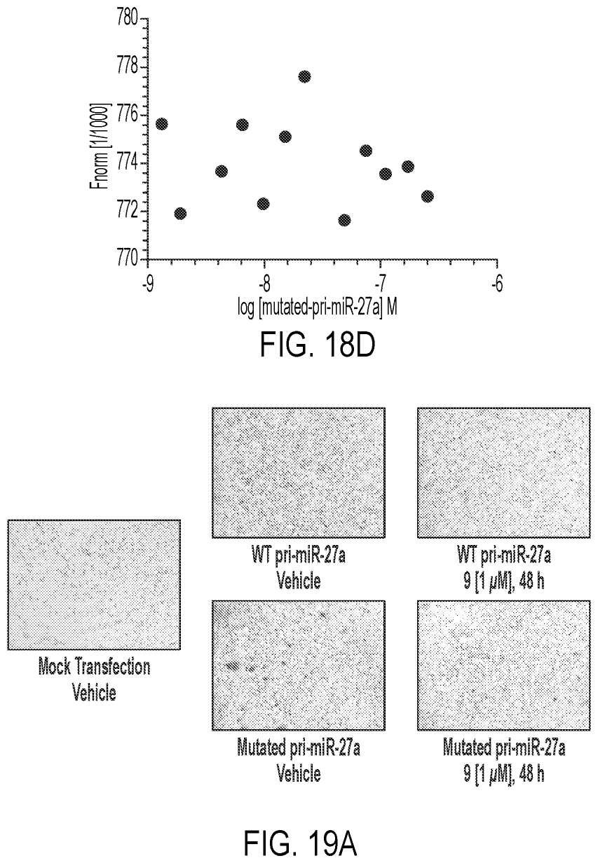

To measure the affinity of 9 for the 5′GAG/3′CCC loop in the context of pri-miR-27a, a competitive binding assay was used with a constant concentration of compound (100 nM) and the Cy5-labeled model of pri-miR-27a's Drosha site, and varying concentrations of unlabeled miR-27a precursor as reported by miRBase. (35) That is, the miR-27a precursor competes with the Cy5-labeled model of pri-miR-27a's Drosha site for binding 9. In this assay, 9 bound to pri-miR-27a with a K_{d} of 40±30 nM; binding was abolished when the two 5′GAG/3′CCC loops were mutated to base pairs (FIG. 18). Thus, the compounds identified by using DEL-2DCS can bind avidly to biologically relevant RNA targets identified by Inforna.

Compound 9 inhibits the cellular processing of pri-miR-27a.

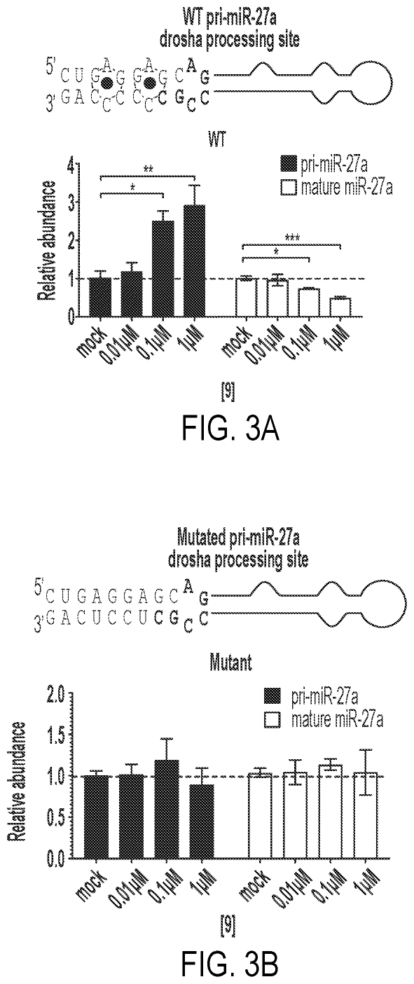

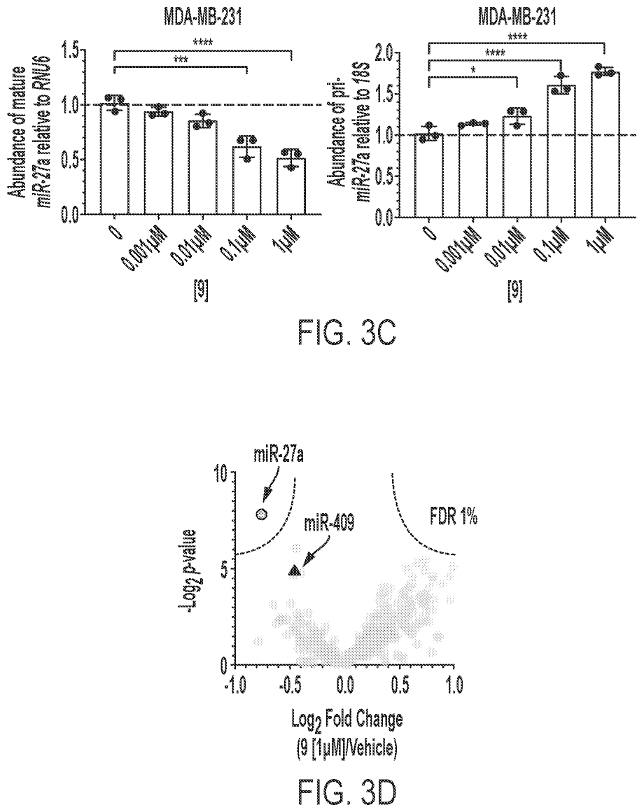

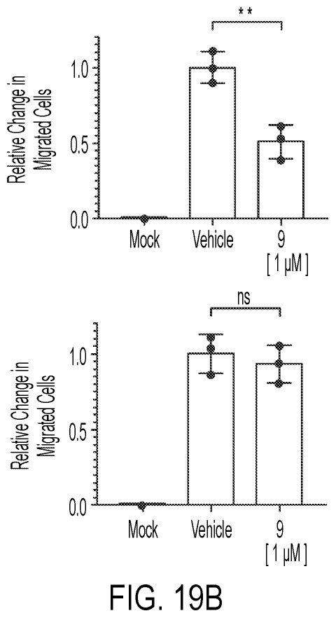

To gauge the specificity of 9 for pri-miR-27a, inhibition of biogenesis was first studied in a cellular model of healthy breast epithelium, MCF-10a, in which miR-27a expression is not detectable (Cₜ>31). MCF-10a cells were transfected with a plasmid encoding WT pri-miR-27a or mutant in which the two 5′GAG/3′CCC internal loops at and nearby the Drosha processing site were mutated to base pairs. In the absence of compound treatment, both primary miRNA transcripts were processed similarly, generating mature miR-27a, as determined by RT-qPCR. Notably, expression of both WT pri-miR-27a and the mutant conferred migratory characteristics to MCF-10a cells, the same phenotype observed in TNBC cells that is caused by miR-27a overexpression (FIG. 19). (36) Importantly, 9 only inhibited the biogenesis of WT pri-miR-27a, as evidenced by reduction of mature miR-27a levels and an increase of pri-miR-27a levels, as determined by RT-qPCR (FIG. 3A). In contrast, 9 did not inhibit biogenesis of the mutated pri-miR-27a in MCF-10A (FIG. 3B) or rescue the migratory phenotype induced by its forced expression (FIG. 19). Collectively, these studies in MCF-10a show that 9 specifically binds the 5′GAG/3′CCC internal loop present in pri-miR-27a's Drosha processing site.

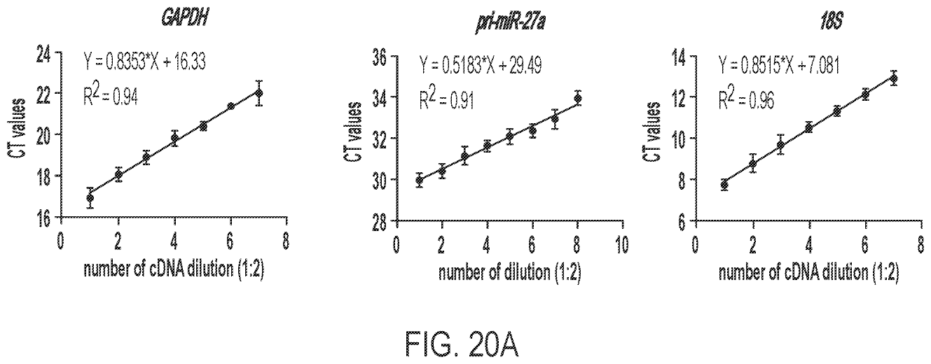

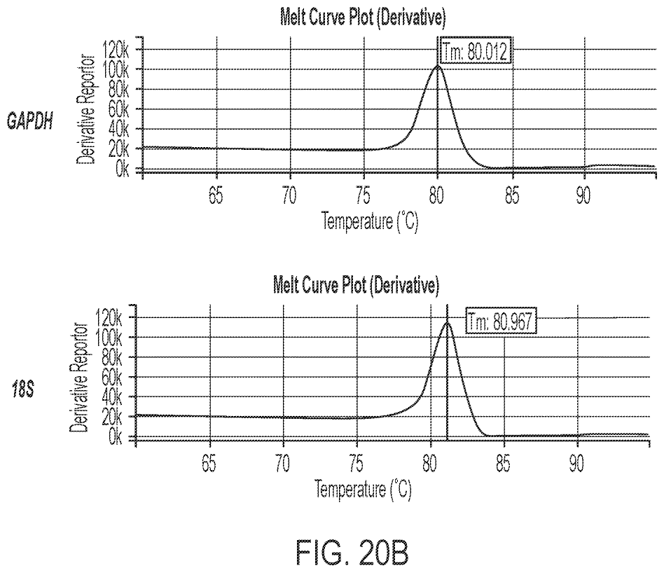

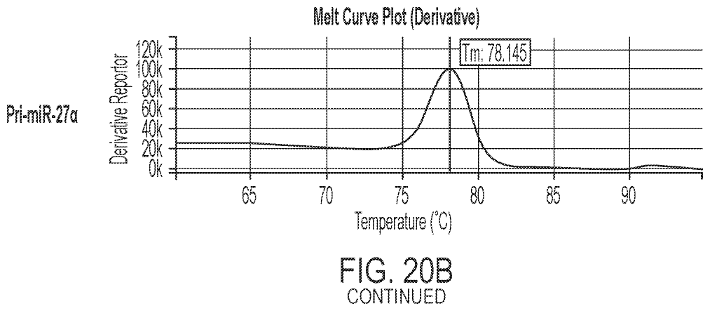

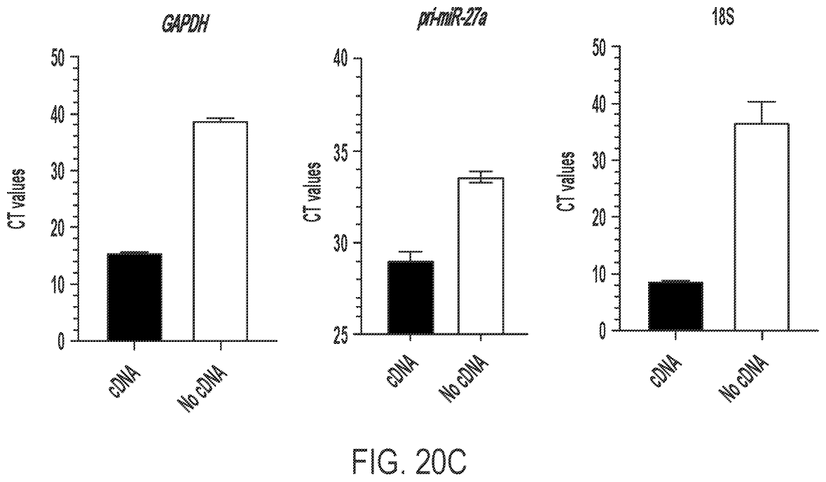

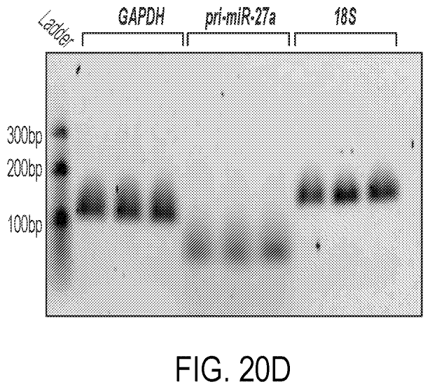

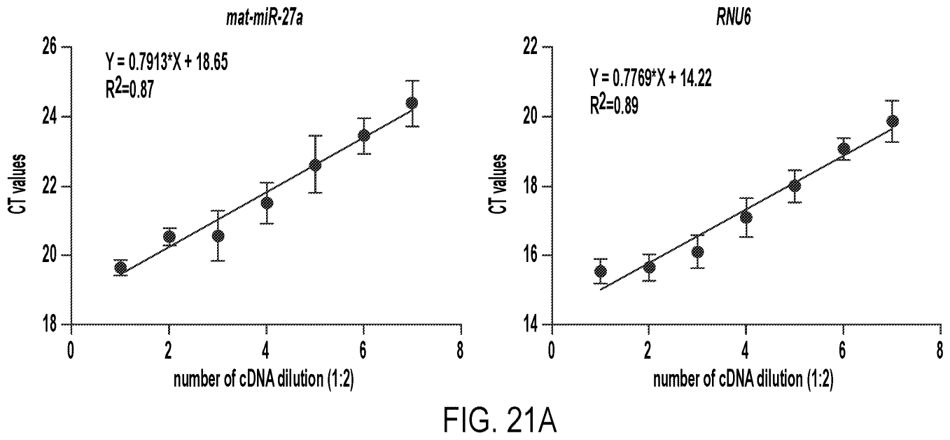

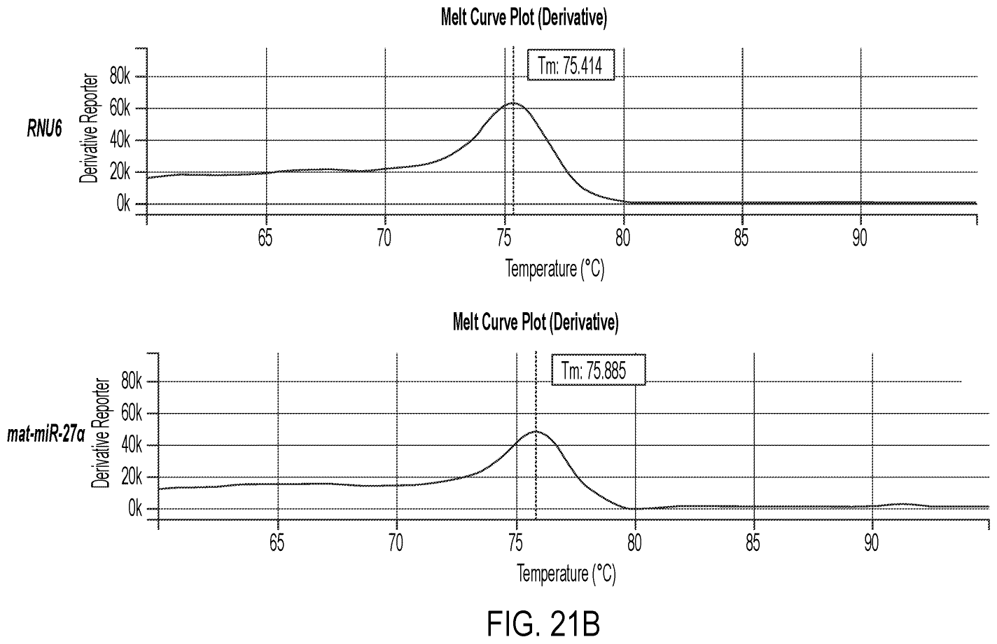

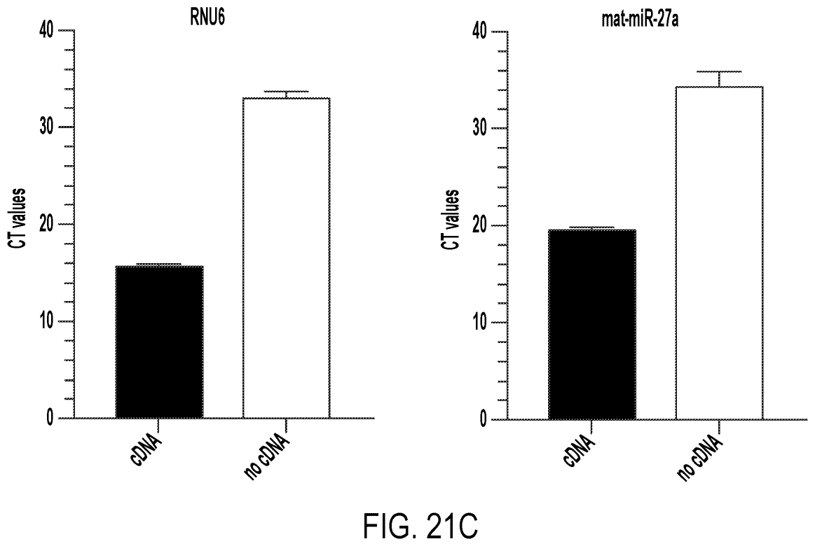

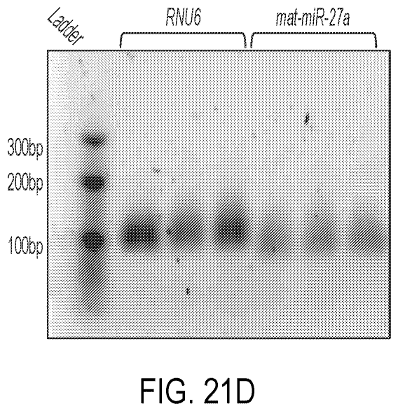

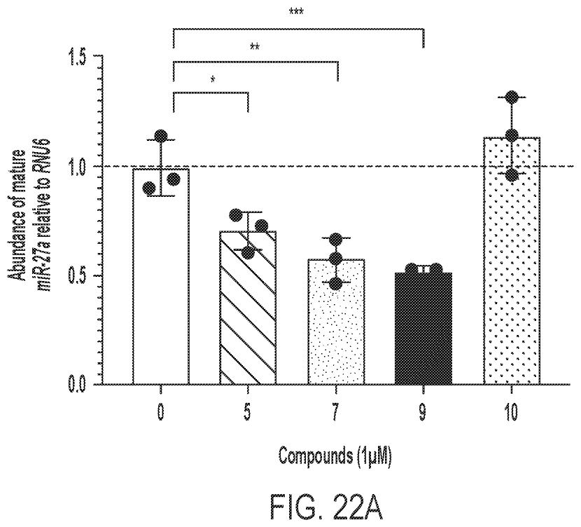

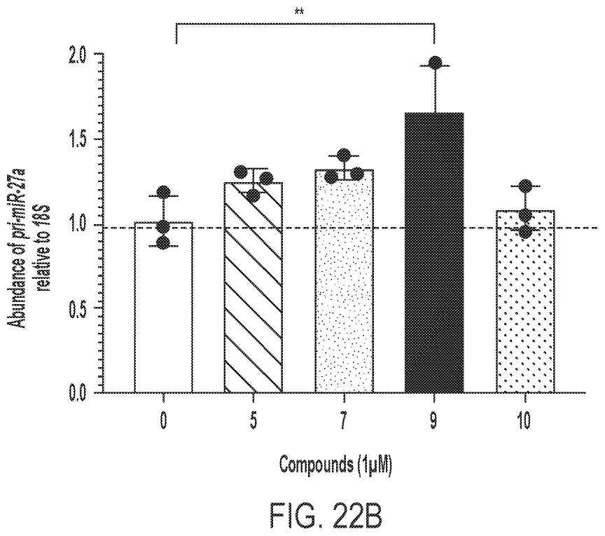

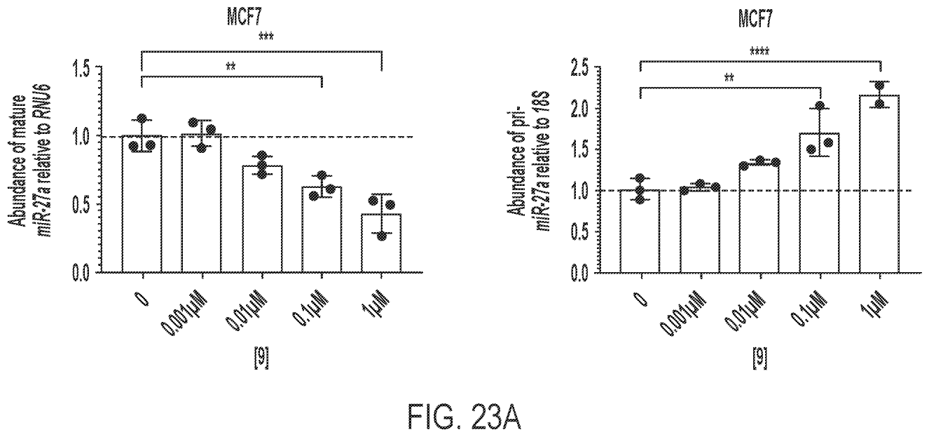

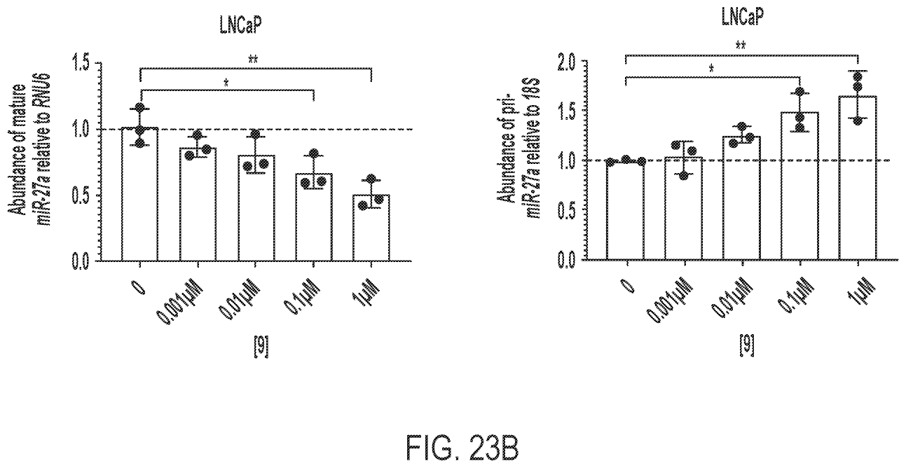

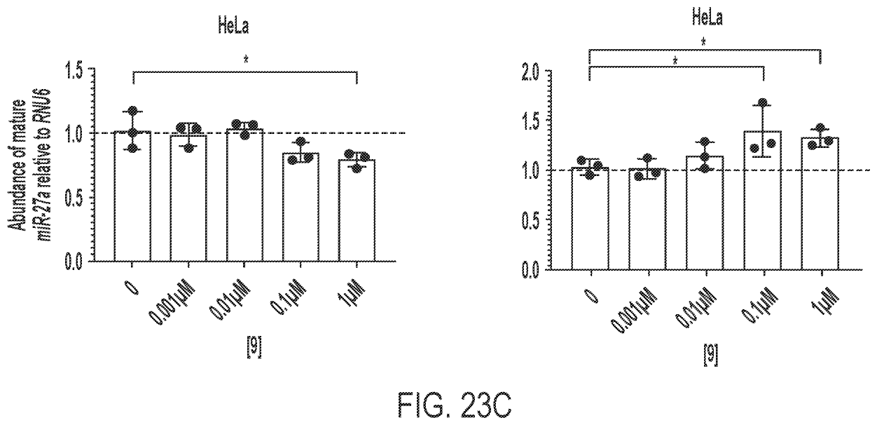

Given these favorable results in MCF-10a cells, a study was undertaken whether 9 could inhibit pri-miR-27a biogenesis, i.e., reduce mature miR-27a abundance, in MDA-MB-231 TNBC cells, in which its overexpression is tied to oncogenesis. (36) In agreement with its putative mode of action, 9 dose dependently reduced mature miR-27a levels with an IC₅₀ of ˜1 μM (FIG. 3C) and increased pri-miR-27a levels, as determined by RT-qPCR (FIG. 3C). Further, in agreement with their in vitro binding affinities, 9 inhibited miR-27a to a greater extent than 5 and 7 while 9's diastereomer, 10, was inactive (FIGS. 20-22). Notably, 9 inhibited miR-27a biogenesis dose dependently as assessed by its effect on pri- and mature miR-27a levels in three other cancer cell lines in which miR-27a is aberrantly expressed: MCF-7 (breast cells), (39) LNCaP (prostate adenocarcinoma). (37) and HeLa (cervical cancer) (40) (FIG. 23). Significant inhibition of miR-27a processing was observed at a dose as low as of 100 nM in all three cancer cell lines (FIG. 23).

Specificity of 9 for the Functional Inhibition of miR-27a

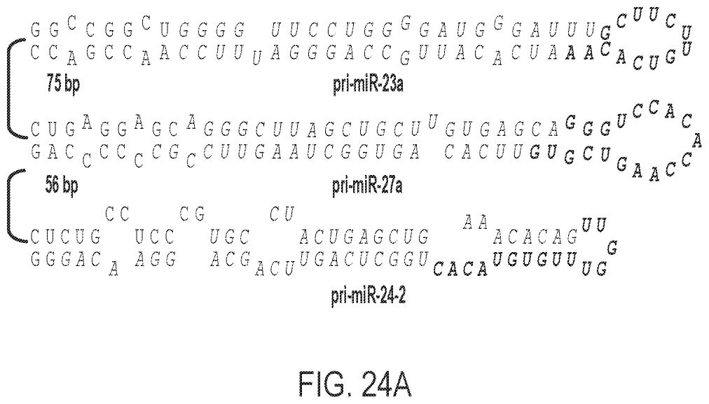

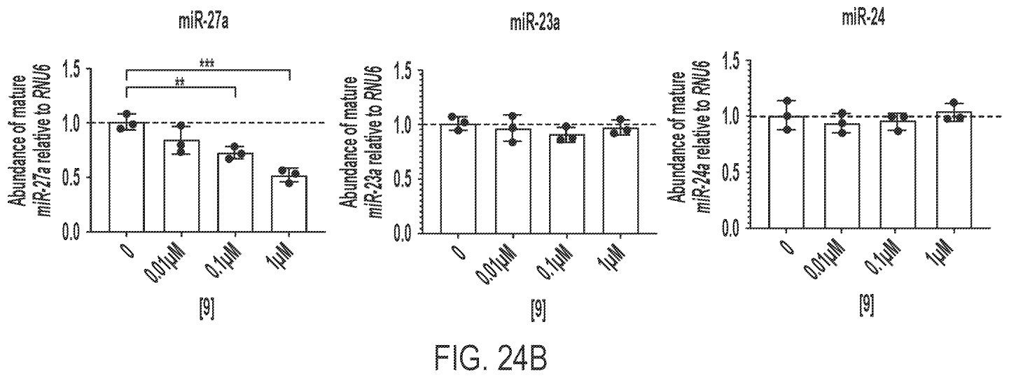

Interestingly, pri-miR-27a is part of a larger pri-miRNA cluster, transcribed as a single transcript along with pri-miR-23a and pri-miR-24, which may act individually or cooperatively to regulate gene expression. (41) The processing of each transcript appears to occur independently as varying expression levels have been observed in different cell types, and miR-27a can be downregulated independently of the other two. (42) For example, previous studies showed that forced expression of the cluster in HEK293T cells increased levels of miR-27a and miR-24-2 but not miR-23a, indicating a block in the processing of pri-miR-23a; (41) this block was not observed in HeLa or P19 (mouse teratocarcinoma) cells. (43) As expected from these previous studies, 9 had no effect on the levels of mature miR-24-2 or miR-23a upon treatment of MDA-MB-231 cells (FIG. 24).

A broader view of 9's selectivity across the miRNome was afforded by profiling the levels of all miRNAs detectable in MDA-MB-231 cells, represented as a volcano plot, a logarithmic plot of fold change as a function of statistical significance (FIG. 3D). The only statistically significant effect observed upon treatment with 1 μM of 9 (false discovery rate (FDR)<1%; P<0.01) was the downregulation of miR-27a. Notably, the levels of miR-409, which has the same Drosha processing site as miR-27a, was not affected, likely due to its significantly lower expression levels in MDA-MB-231 cells (128-fold less abundant) and hence lower cellular occupancy by 9. Furthermore, the abundance of other oncogenic miRNAs, such as miR-96, miR-155, and miR-21 known to be also correlated with TNBC, were not affected (FIG. 3D). (44-46)

Effect of 9 on the proteome

As the miRNome-wide studies indicated that 9 is specific for inhibition of miR-27a biogenesis, an investigation was undertaken whether these effects translated to the proteome. Notably, miR-27a has been implicated in the regulation of various pathways, including oncogenesis. (47, 48) In particular, Zinc Finger And BTB Domain Containing 10 (ZBTB10 aka RINZF) is known to be down-regulated by miR-27a in breast cancer cells, particularly MDA-MB-231 cells. (41, 48) Additionally, TargetScan (49) predicts that Protein Phosphatase 4 Catalytic (PP4C) and Programmed Cell Death 4 (PDCD4) are direct targets of miR-27a, both of which have been directly correlated with migratory functions of breast cancer cell lines. (50, 51)

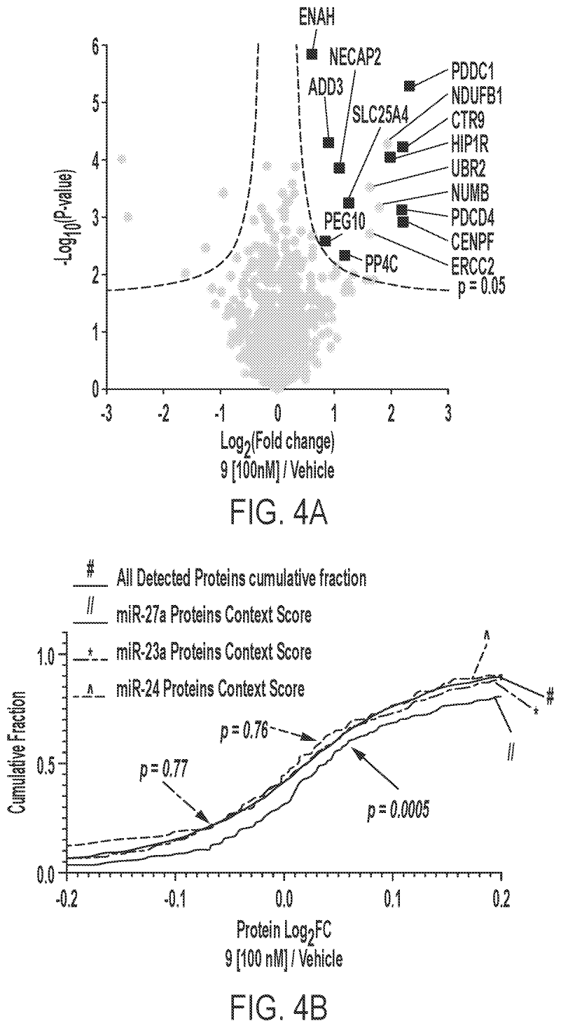

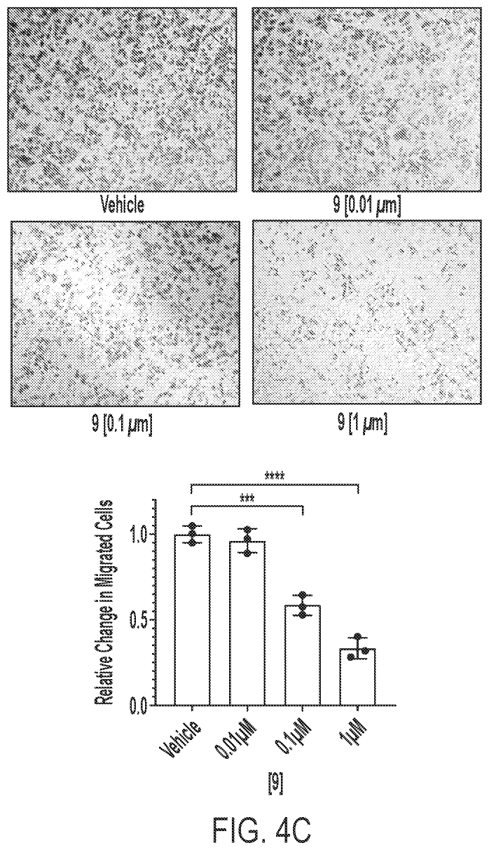

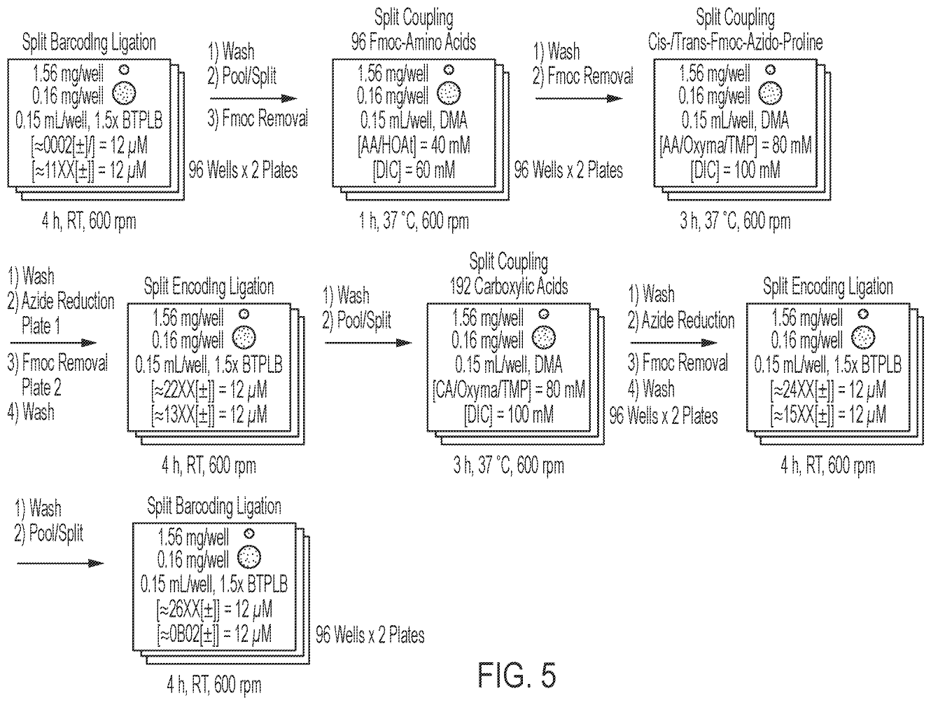

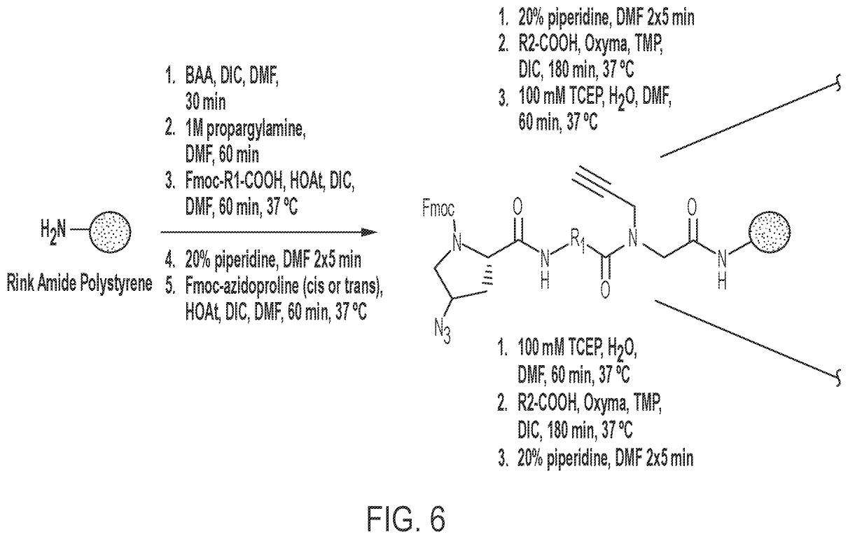

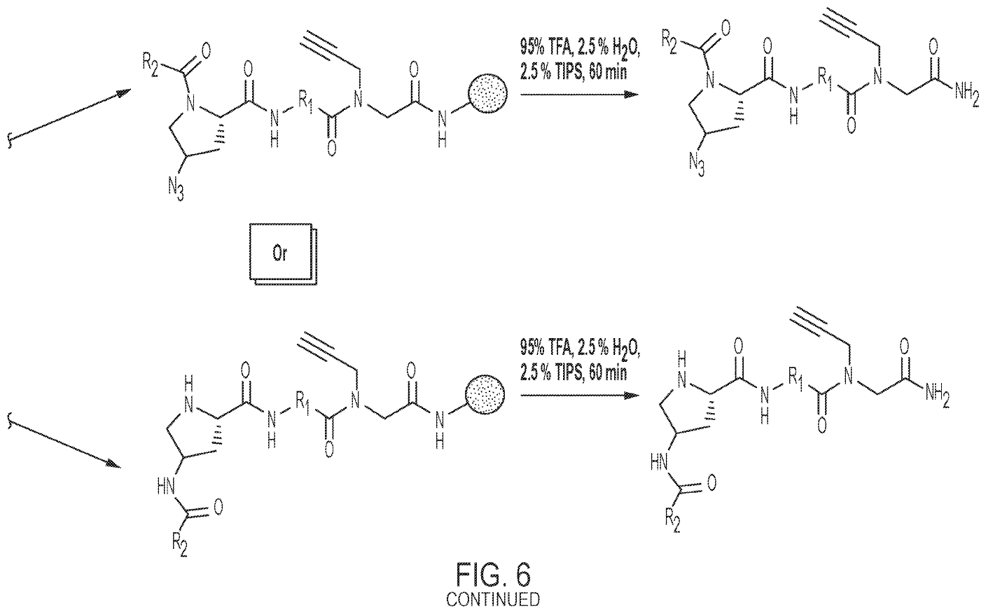

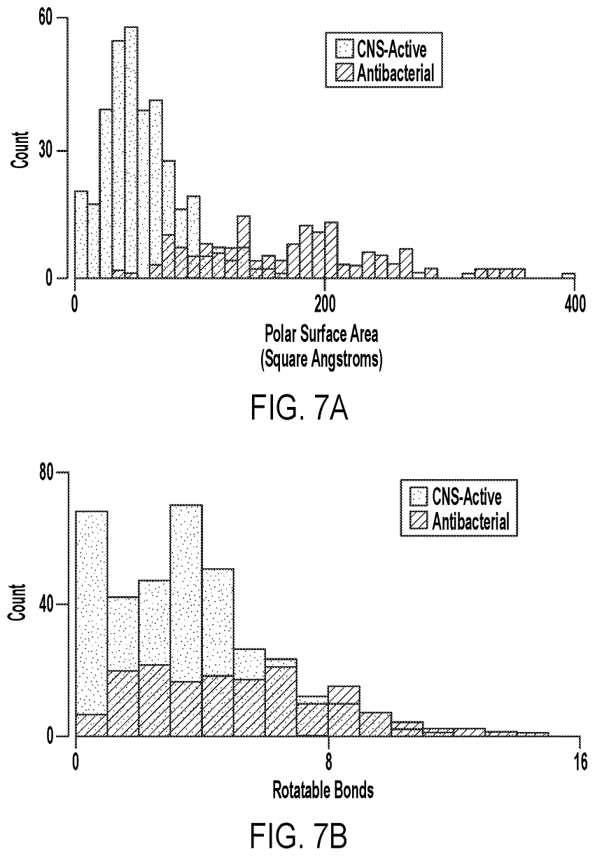

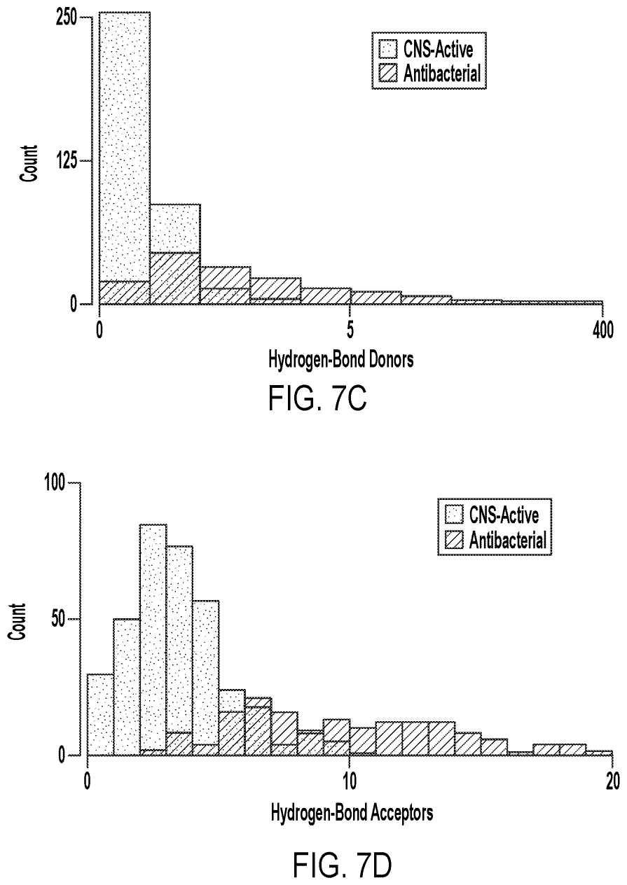

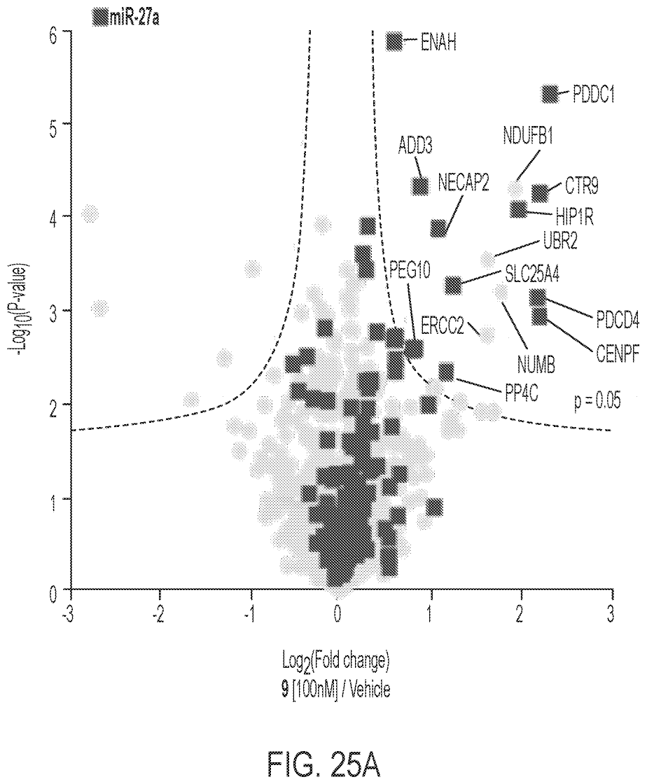

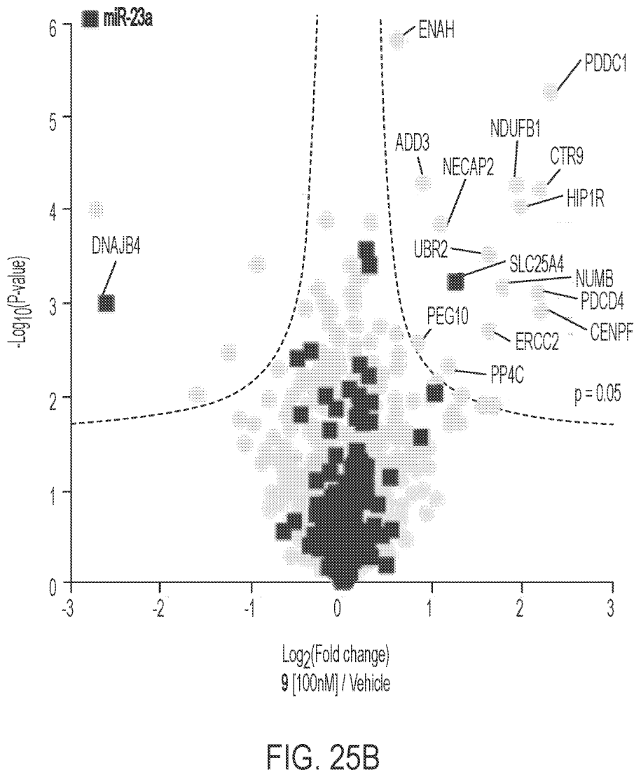

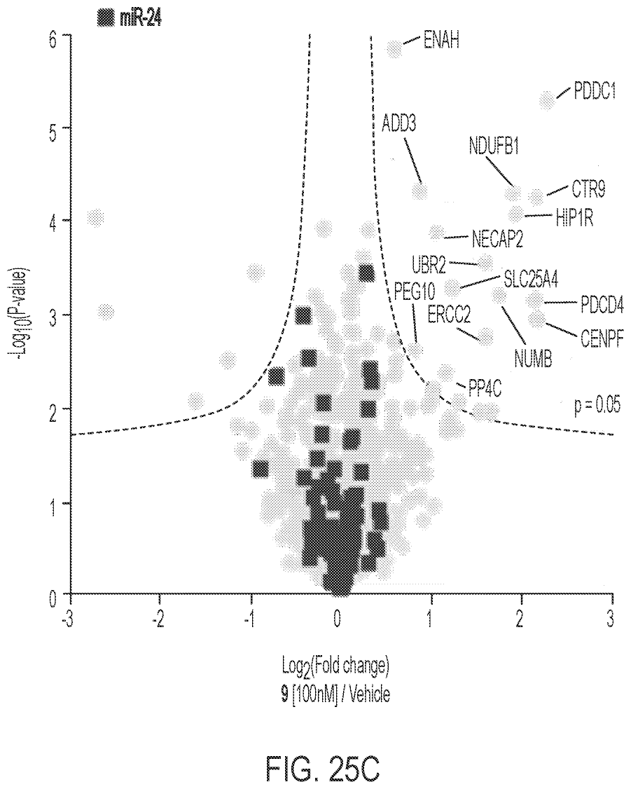

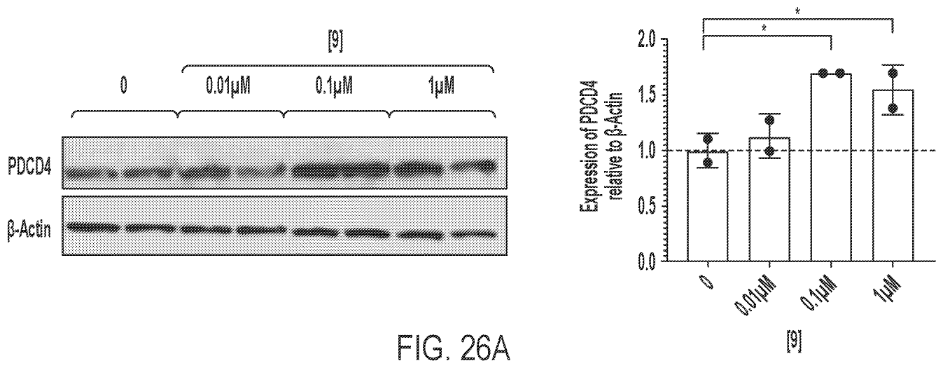

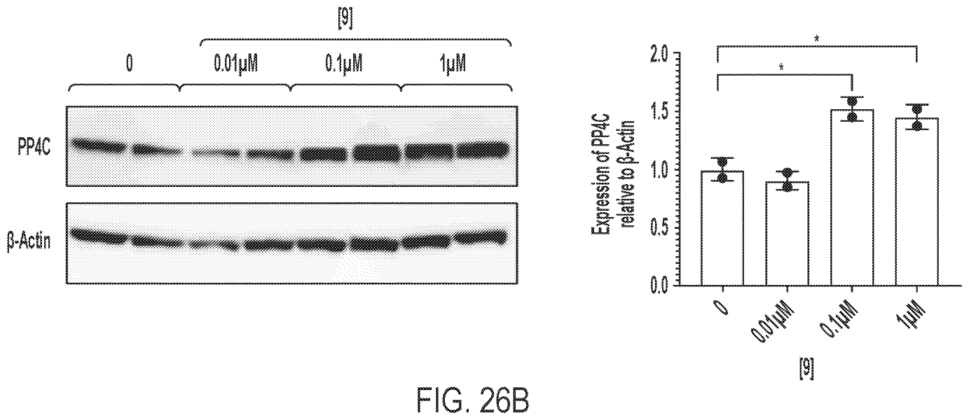

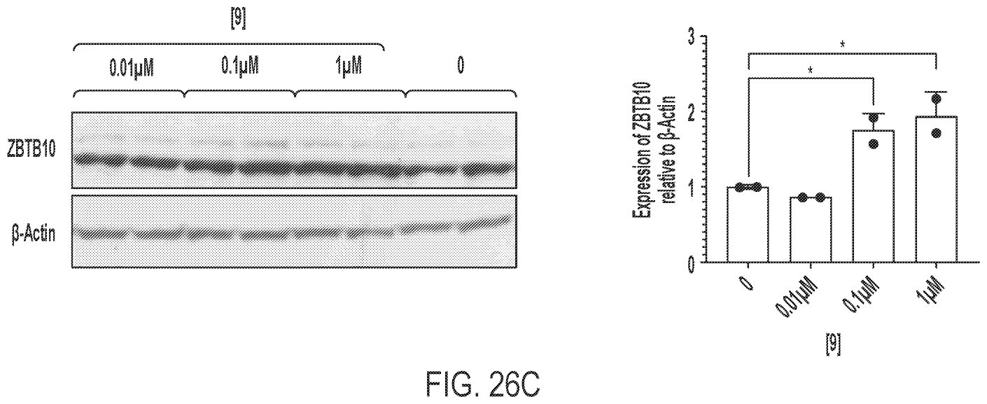

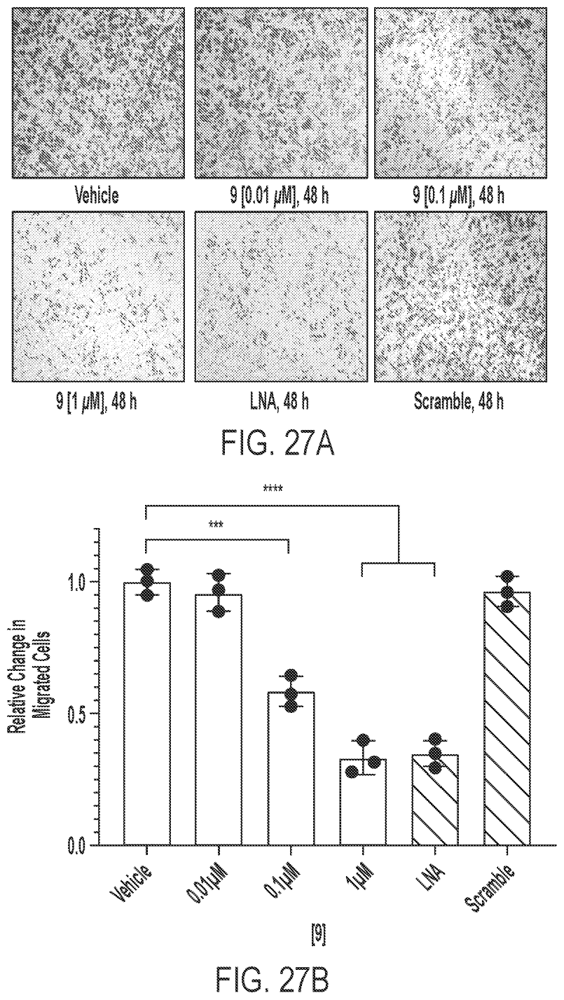

To investigate the overall effect of 9 on the proteome including miR-27a's direct targets, MDA-MB-231 cells were treated with 100 nM of the compound and subjected to global proteomics analysis as previously described. (52) Of the 3,127 detectable proteins, only 15 were significantly upregulated and five significantly downregulated (FDR <5%; FIG. 4A). Eleven of the 15 upregulated proteins are putative direct targets of miR-27a [as per TargetScan (49)] including ENAH Actin Regulator (ENAH), Adducin 3 (ADD3), NECAP Endocytosis Associated 2 (NECAP2), Paternally Expressed 10 (PEG10), PP4C, and PDCD4. Only one of these upregulated proteins is directly targeted by miR-23a (Solute Carrier Family 25 Member 4; SLC25A4) or miR-24 (FIG. 25), however this protein is also directly regulated by miR-27a (FIG. 25). The upregulation of PP4C, PDCD4, and ZBTB 10 was confirmed by Western blotting (FIG. 26), which mirrored the dose dependent reduction of mature miR-27a levels.

Next the changes in expression levels were analyzed across the entire proteome to quantify the effects of 9 on protein levels encoded by mRNAs that are regulated by miR-27a, miR-24, or miR-23a, as determined by the corresponding context score reported by TargetScan. (53) This analysis showed that the changes observed proteome-wide were correlated with the targets of miR-27a but not with the targets of miR-24 or miR-23a (FIGS. 4-B & 25).

As the proteomics data indicated a specific effect on miR-27a regulated proteins, the effect of 9 on phenotype, namely the migratory mature of MDA-MB-231 cells, was assessed. A dose dependent reduction in the number of migrated cells was observed upon treatment with 9, with statistically significant phenotype reduction using as little as 100 nM small molecule (FIGS. 4-C and D, & 27). In conjunction with studies in MCF-10a cells forced to express WT or mutant pri-miR-27a (FIG. 3), these studies collectively demonstrate rescue of the miR-27a-mediated oncogenic phenotype by 9.

The above described screening results according to the invention demonstrate that the DEL-2DCS approach delivers deliver a trove of structures with unexpected propensity to bind nucleic acids functionally in cells. Many RNA-binding molecules have tended to be flat, interacting with nucleic acids via x stacking interactions, although there are many examples of groove binders as well. The present invention shows that these properties are not necessarily dominant considerations for RNA binding. Although many of the DEL hits according to the invention contained heavily conjugated and electron rich x systems evocative of nucleic acid stains, the most prevalent building block, the Freidinger's lactam, lacks such features. Furthermore, it has been found that small molecule diastereomer pairs exhibit markedly different RNA target binding fingerprints.

According to the invention, the combinatorial approach of DEL and RNA targets yielded novel bioactive ligands that inhibited processing of miR-27a, an important oncogenic miRNA associated with various cancers. The lead molecule, 9, (pc9(S)) bound miR-27a with nanomolar affinity, targeting an internal loop pocket present twice within the Drosha processing site. Compound 9 significantly decreased miR-27a expression in four different cancer cell lines at nanomolar concentrations with high selectivity across the miRNome. The inhibition of its biogenesis selectively induced the de-repression of associated proteins and the diminution of the migratory phenotype associated with breast cancer disease.

### Mechanism of Action and Medical Treatment

In certain embodiments, the invention is directed to methods of inhibiting, suppressing, derepressing and/or managing biolevels of pri-miR-27a present in oncologic cell lines and in animals and humans having such oncologic cells and. The peptide compounds pc5 (s), pc7(S) and pd9 (s) and especially the peptide compound pd9 (s) as embodiments of the invention for use in the methods disclosed herein bind to the above identified RNA entity as well in the above identified cell lines, animals and humans.

Embodiments of the Compounds applied in methods of the invention and their pharmaceutical compositions are capable of acting as “inhibitors”, suppressors and or modulators of the above identified RNA entities which means that they are capable of blocking, suppressing or reducing the expression of the RNA entities. An inhibitor can act with competitive, uncompetitive, or noncompetitive inhibition. An inhibitor can bind reversibly or irreversibly.

The compounds useful for methods of the invention and their pharmaceutical compositions function as therapeutic agents in that they are capable of preventing. ameliorating, modifying and/or affecting a disorder or condition. The characterization of such compounds as therapeutic agents means that, in a statistical sample, the compounds reduce the occurrence of the disorder or condition in the treated sample relative to an untreated control sample, or delays the onset or reduces the severity of one or more symptoms of the disorder or condition relative to the untreated control sample.

The ability to prevent, ameliorate, modify and/or affect in relation to a condition, such as a local recurrence (e.g., pain), a disease known as a polycystic disease including but not limited to polycystic kidney disease or an oncologic disease such as but not limited to breast cancer and/or prostate cancer or any other neoplastic and/or oncologic disease or condition, especially having etiology similar to breast and/or prostate cancer may be accomplished according to the embodiments of the methods of the invention and includes administration of a composition as described above which reduces, or delays or inhibits or retards the oncologic medical condition in a subject relative to a subject which does not receive the composition.

The compounds of the invention and their pharmaceutical compositions are capable of functioning prophylactically and/or therapeutically and include administration to the host/patient of one or more of the subject compositions. If it is administered prior to clinical manifestation of the unwanted condition (e.g., disease or other unwanted state of the host animal/patient) then the treatment is prophylactic, (i.e., it protects the host against developing the unwanted condition), whereas if it is administered after manifestation of the unwanted condition, the treatment is therapeutic, (i.e. it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated, e.g., at a reasonable benefit/risk ratio applicable to any medical treatment.

### Administration

Compounds of the invention and their pharmaceutical compositions prepared as described herein can be administered according to the methods described herein through use of various forms, depending on the disorder to be treated and the age, condition, and body weight of the patient, as is well known in the art. As is consistent, recommended and required by medical authorities and the governmental registration authority for pharmaceuticals, administration is ultimately provided under the guidance and prescription of an attending physician whose wisdom, experience and knowledge control patient treatment.

For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route or other similar transmucosal route, they may be formulated as drops or ointments.

These formulations for administration orally or by a transmucosal route can be prepared by conventional means, and if desired, the active ingredient may be mixed with any conventional additive or excipient, such as a binder, a disintegrating agent, a lubricant, a corrigent, a solubilizing agent, a suspension aid, an emulsifying agent, a coating agent, a cyclodextrin, and/or a buffer. Although the dosage will vary depending on the symptoms, age and body weight of the patient, the gender of the patient, the nature and severity of the disorder to be treated or prevented, the route of administration and the form of the drug, in general, a daily dosage of from 0.0001 to 2000 mg, preferably 0.001 to 1000 mg, more preferably 0.001 to 500 mg, especially more preferably 0.001 to 250 mg, most preferably 0.001 to 150 mg of the compound is recommended for an adult human patient, and this may be administered in a single dose or in divided doses. Alternatively, a daily dose can be given according to body weight such as 1 nanogram/kg (ng/kg) to 200 mg/kg, preferably 10 ng/kg to 100 mg/kg, more preferably 10 ng/kg to 10 mg/kg, most preferably 10 ng/kg to 1 mg/kg. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those excipients, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutical Compositions Incorporating Compounds pc5 (s), pc7 (s) and pc9(S) and Especially pc9(S)

The pharmaceutical compositions of the invention incorporate embodiments of Compounds pc5 (s), pc7 (s) and pc9(S) useful for methods of the invention and a pharmaceutically acceptable carrier. The compositions and their pharmaceutical compositions can be administered orally, topically, parenterally, by inhalation or spray or rectally in dosage unit formulations. The term parenteral is described in detail below. The nature of the pharmaceutical carrier and the dose of these Compounds depend upon the route of administration chosen, the effective dose for such a route and the wisdom and experience of the attending physician.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
(3) humectants, such as glycerol;
(4) disintegrating agents, such as agar-agar, calcium carbonate, potato or tapioca starch, alginic acid, certain silicates, and sodium carbonate;
(5) solution retarding agents, such as paraffin;
(6) absorption accelerators, such as quaternary ammonium compounds;
(7) wetting agents, such as, for example, acetyl alcohol and glycerol monostearate;
(8) absorbents, such as kaolin and bentonite clay;
(9) lubricants, such a talc, calcium stearate, magnesium stearate, solid polyethylene glycols, sodium lauryl sulfate, and mixtures thereof; and
(10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using. for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound useful for application of methods of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound useful for practice of methods of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) useful for application of the methods of the invention may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compound(s) useful for application of methods of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) useful for application of methods of the invention in the pharmaceutical compositions of this invention may be varied to obtain an amount of the active ingredient which is effective to achieve the desired therapeutic response for a particular patient, composition, and mode of administration, without being toxic to the patient.

The concentration of a compound useful for application of methods of the invention in a pharmaceutically acceptable mixture will vary depending on several factors, including the dosage of the compound to be administered, the pharmacokinetic characteristics of the compound(s) employed, and the route of administration.

In general, the compositions useful for application of methods of this invention may be provided in an aqueous solution containing about 0.1-10% w/v of a compound disclosed herein, among other substances, for parenteral administration. Typical dose ranges are those given above and may preferably be from about 0.001 to about 500 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

### Experimental Section Methods

Abbreviations. ACN: acetonitrile, BP: base paired, BSA: bovine serum albumin, COMU: 1-Cyano-2-ethoxy-2-oxoethylidenaminooxy) dimethylamino-morpholino-carbenium hexafluorophosphate, DCM: dichloromethane, DIC: diisopropylcarbodiimide, DIEA: N,N-diisopropylethylamine, DI H₂O: deionized water, DMA: dimethylacetamide, DMEM: Gibco Dulbecco's Modified Eagle Medium, DMF: N, N-dimethylformamide, DMSO: dimethylsulfoxide, DPBS: Dulbecco's phosphate buffered saline, EDTA: ethylenediaminetetraacetic acid, FA: formic acid, FBS: fetal bovine serum, Fmoc: fluorenylmethyloxycarbonyl, FDR: false discovery rate, HATU: 1-[Bis(dimethylamino)methylene]-1H-1,2,3-triazolo[4,5-b]pyridinium 3-oxide hexafluorophosphate, HCCA: α-cyano-4-hydroxycinnamic acid, HDNA: DNA headpiece, HEPES: 4-(2-hydroxyethyl)-1-piperazineethanesulfonic acid, HOAt: 1-Hydroxy-7-azabenzotriazole, HPLC: high-performance liquid chromatography, HRMS: high-resolution mass spectrometry, ILL: internal loop library, LC: liquid chromatography, LC-MS/MS: liquid chromatography coupled with tandem mass spectrometry, MeOH: methanol, MS: mass spectrometry, MST: microscale thermophoresis, NGS: next generation sequencing, OP stock: oligonucleotide paired stock, PAGE: polyacrylamide gel electrophoresis, PBS: phosphate buffered saline, PCR: polymerase chain reaction, PFA: paraformaldehyde, PTFE: polytetrafluoroethylene, QC: quality control, RPMI: Roswell Park Memorial Institute formulation. SDS: sodium dodecyl sulfate, TBS: tris buffered saline, TBST: tris buffered saline with 0.05% (v/v) Tween-20, TCEP: tris(2-carboxyethyl) phosphine, THPTA: Tris(3-hydroxypropyltriazolylmethyl)amine, TIPS: triisopropylsilane, TMP: 2,4,6-trimethylpyridine, TFA: trifluoroacetic acid, UMI: unique molecular identifier.

Mass Spectrometry. Matrix-assisted laser desorption ionization time-of-flight (MALDI-TOF) mass spectrometry was performed on an AB SCIEX 4800 Plus MALDI-TOF/TOF instrument using α-cyano-4-hydroxycinnamic acid as matrix. Spectra were acquired using the 4000 Series Explorer software (AbSciex, v 3.2.3) and analyzed using open-source software mMass (v 5.5.0). HRMS spectra were collected by internal calibration using the Mass Standards Kit for Calibration of AB Sciex TOF/TOF TM Instruments (P/N 4333604).

Analytical HPLC. HPLC analyses were conducted on a system composed of Waters 2487 Dual Absorbance Detector and Waters 1525 Binary HPLC Pump equipped with a Sunfire C18 4.8×150 mm column. Analyses were conducted with a flow rate of 1 mL/min with a gradient of 0-100% MeOH (+0.1% TFA) in water (+0.1% TFA) over 40 min followed by 5 min at 100% MeOH (+0.1% TFA).

Preparative HPLC. HPLC analyses were conducted on a system composed of Waters 2487 Dual Absorbance Detector, Waters 1525 Binary HPLC Pump and Waters Fraction Collector III. The system was equipped with a Sunfire PREP C18 19×150 mm. Analyses were conducted with a flow rate of 5 mL/min with a gradient of 0-100% MeOH (+0.1% TFA) in water (+0.1% TFA) over 40 min followed by 5 min at 100% MeOH (+0.1% TFA).

### Solid-Phase Del Synthesis & Screening

Materials Sources. All reagents were obtained from MilliporeSigma (St. Louis, MO) unless otherwise specified: 1.3-Bis[tris(hydroxymethyl)methylamino] propane (Bis-Tris), trifluoroacetic acid (TFA), triisopropylsilane (TIPS), tris(2-carboxyethyl) phosphine (TCEP), α-cyano-4-hydroxycinnamic acid (HCCA) (Life Technologies, Carlsbad, CA), N,N′-diisopropylcarbodiimide (DIC, Acros Organics, Fair Lawn, NJ), 1-hydroxy-7-azabenzotriazole (HOAt, Accela ChemBio Inc., San Diego, CA), 2,4,6-trimethylpyridine (TMP), ethyl cyanohydroxyiminoacetate (Oxyma), dimethylformamide (DMF, Thermo Fisher Scientific, Waltham, MA), dichloromethane (DCM, Thermo Fisher Scientific), N,N-diisopropylethylamine (DIEA, Thermo Fisher Scientific), acetonitrile (can, Thermo Fisher Scientific), dimethyl sulfoxide (DMSO, AMRESCO Inc., Solon, OH), (4-Fmoc-2-methoxy-5-nitrophenoxy) butanoic acid (Fmoc-PC-OH, Santa Cruz Biotechnology Inc., Dallas, TX), N-α-Fmoc-N-8-7-methoxycoumarin-4-acetyl-L-lysine (N-α-Fmoc-K (Mca)-OH), Nα-Fmoc-Nω-(2,2,4,6,7-pentamethyldihydrobenzofuran-5-sulfonyl)-L-arginine (N-α-Fmoc-R (Pbf)-OH, Thermo Fisher Scientific), sodium acetate, (GPR, CPC Scientific, Sunnyvale, CA), calcium chloride, Taq DNA polymerase (Taq. New England Biolabs, Ipswich, MA), and 2′-deoxyribonucleotide triphosphate (dNTP, set of dATP, dTTP, dGTP, dCTP, Promega Corp., Milwaukee, WI), were used as provided. Solvents used in solid-phase synthesis were dried over molecular sieves (3 Å, 3.2 mm pellets).

Oligonucleotides (Integrated DNA Technologies, Inc. Coralville, IA) were purchased as desalted lyophilate and used without further purification. Oligonucleotide ligation substrates were 5′-phosphorylated (/5Phos/). The amino-modified precursor DNA piece used for conjugation on beads (NH₂-HDNA; /5Phos/GAGTCA/iSp9//iUniAmM//iSp9/TGACTCCC iSp9 indicates a 9 atom triethylene glycol spacer and iUniAmM indicates an amino-modified six carbon aliphatic spacer) was HPLC purified by the manufacturer and used without further purification. [Note: the HDNA is transformed to a clickable N₃-modified oligonucleotide via acylation w/azidopentanoic acid.]

Buffers. Bis-Tris propane Wash Buffer (BTPWB, 50 mM NaCl, 0.04% Tween-20, 10 mM Bis-Tris, pH 7.6); 10× Bis-Tris propane Ligation Buffer (BTPLB, 500 mM NaCl, 100 mM MgCl₂, 10 mM ATP, 0.2% Tween-20, 100 mM Bis-Tris propane, pH 7.6); 10× PCR Buffer (2 mM dATP, 2 mM dGTP, 2 mM dCTP, 2 mM dTTP, 15 mM MgCl₂, 500 mM KCl, 100 mM Tris, pH 8.3), 1× GC-PCR Buffer (1× PCR buffer, 8% (v/v) DMSO, 1 M betaine), and Crush and Soak buffer (C&S, 50 mM NaCl, 10 mM Tris —HCl pH 7.5, 1 mM EDTA). For DEL synthesis, buffers were prepared in deionized water. Otherwise, buffers were prepared in Nanopure H₂O.

Bifunctional library resin synthesis and characterization. The azido-modified headpiece DNA (N₃-HDNA) was prepared as previously described. (5) Linker synthesis proceeded via iterative cycles of solid-phase synthesis. All spin-column wash and reaction volumes were identical (0.4 mL) unless noted. All fritted-syringe wash and reaction volumes were identical (3.0 mL) unless noted. All filtration microplate wash and reaction volumes were identical (0.15 mL) unless otherwise noted.

Quality-control (QC) TentaGel rink amide resin (160 μm, 0.40 mmol/g, 50 mg, Rapp-Polymere, Tuebingen, Germany) was transferred to a fritted spin-column (Mobicol, large filter. 10-μm pore size), swelled in solvent (DMF, 16 h, room temperature, 8 rpm), and washed (3×DMF). Fmoc was removed (20% piperidine in DMF, 1×5 min, 1×15 min, room temperature, 8 rpm), and the resin was washed (3×DMF, 3×DCM, 3×DMF). Then, N-α-Fmoc-K (Mca)-OH (60 μmol) was activated (2 min, room temperature) with COMU/DIEA (60/120 μmol) in DMF, added to resin, and the resin was incubated (30 min, 50° C. 8 rpm, 2×). After washing the resin (3×DMF, 3×DCM, 3×DMF), N-α-Fmoc-R (Pbf)-OH (60 μmol; 2 min, room temperature) activated with COMU/DIEA (60/120 μmol) in DMF was added to resin, and the resin was incubated (30 min, 50° C., 8 rpm). The resin was washed (3×DMF, 3×DCM, 3×DMF), unreacted sites were acetylated (20% acetic anhydride in DMF, 15 min, 50° C., 8 rpm), and the resin was washed again (6×DMF, 6×DCM, 3×DMF).

Synthesis resin (amino-functionalized, 10 μm dia., 0.29 mmol/g, 300 mg, Rapp-Polymere) and the aforementioned 160-μm QC resin (30 mg) were transferred to a syringe (6 mL) equipped with a frit (10-μm polyethylene, 13 mm dia., Biotage, Charlotte, NC), swelled in solvent (DMF, 16 h, room temperature, 8 rpm), and washed (3×DCM, 3×DMF). Subsequent amino acid coupling cycles consisted of: (1) Fmoc removal (20% piperidine in DMF. 1×5 min, 1×15 min, room temperature, 8 rpm); (2)N-α-Fmoc-amino acid (1 mmol) activation with DIC/Oxyma/DIEA (1/1/2 mmol, 2 min, room temperature); (3) addition of activated N-α-Fmoc-amino acid to resin and incubation (1 h, 50° C., 8 rpm). The N-substituted glycine coupling cycle consisted of: (1) Fmoc removal (20% piperidine in DMF, 1 ×5 min, 1×15 min, room temperature, 8 rpm); (2) bromoacetic acid (1 mmol) activation with DIC (2 mmol, 2 min, room temperature); (3) coupling of activated bromoacetic acid to resin (1 h. 50° C., 8 rpm); (4) displacement (1 M propargylamine, 3 h, 50° C., 8 rpm). Unless specified otherwise, following each Fmoc removal and building block coupling step, resin was washed (3×DMF, 3×DCM, 3×DMF). N-α-Fmoc-Gly-OH, bromoacetic acid/propargylamine, and N-α-Fmoc-Gly-OH were coupled sequentially as described above, but without removing Fmoc from the pendant glycine. Fmoc-PC-OH (0.5 mmol) was activated with DIC/Oxyma/TMP (0.75/0.5/0.5 mmol), added to resin, incubated (1×2 h, 1×1 h, 37° C., 8 rpm), and resin was washed (3×DMF, 3×DCM, 3×DMF). Mixed-scale bifunctional-HDNA library resin was prepared and characterized as previously described. (5)

DNA-encoded library resin barcoding. All library synthesis and library handling was performed in a UV-free room. A general protocol for DNA-encoded solid-phase synthesis (DESPS) has previously been described. (5) Oligonucleotides are indicated in bold with the “≈” designation. Numeric identifiers were described previously. (5) Sequences used for DEL construction are listed in Table 2. Oligonucleotide paired (OP) stock solutions of complementary oligonucleotides (60 μM [+], 60 M [−], 50 mM NaCl, 1 mM Bis-Tris pH 7.6) were heated (5 min, 60° C.) and cooled to ambient (5 min, room temperature) before each use. OP stocks bear the [±] designation, indicating “double-stranded.” DEL barcoding and encoded combinatorial synthesis procedures are summarized in Scheme 1.

Ligation of ≈0002 and barcoding ≈11XX oligonucleotide. Mixed-scale bifunctional HDNA library resin was split into 192 wells (0.16 mg 160-μm resin, 63 nmol; 1.56 mg 10-μm resin, 450 nmol) of pre-wetted (3×DCM, 3×DMF) filtration microplates (2×, Millipore MultiScreen Solvinert 0.45 μm Hydrophobic PTFE), washed (3×DMF, 3×1:1 DMF: BTPWB, 3×BTPWB), resuspended (BTPWB), covered with adhesive foil (VWR International, Radnor, PA), incubated (1 h, room temperature, 600 rpm), washed (3×BTPWB, 1×BTPLB), resuspended (BTPLB, 0.1 mL), and incubated while the encoding oligonucleotide ligation mixtures were prepared (˜30 min, room temperature). An encoding oligonucleotide ligation mixture containing ≈0002 [±] (370 nmol), and T4 DNA ligase (550 μg) in 2.25X BTPLB (20.3 mL) was prepared and aliquoted into all plate wells (100 μL) along with DI H₂O (38 μL). OP stocks of ˜11XX [±] (1.8 nmol, 12 μL) were added to the appropriate wells, and the plate was sealed with adhesive foil and incubated (4 h, room temperature, 600 rpm). Resin was washed (3×BTPWB), resuspended (BTPWB, 0.15 mL) and incubated (16 h, room temperature, 600 rpm).

DNA-encoded solid-phase combinatorial library synthesis. Barcoded mixed-scale library resin was retrieved, washed (3×BTPWB, 3×1:1 DMF: BTPWB, 3×DMF), resuspended (DMF, 0.1 mL), pooled into a reservoir, split into 192 wells (0.16 mg 160-μm resin, 63 nmol; 1.56 mg 10-μm resin, 450 nmol) of 2 fresh pre-wetted (3×DCM, 3×DMF) filtration microplates, and washed (2×DMF). Fmoc was removed (20% piperidine in DMF, 1×5 min, 1×15 min, room temperature, 600 rpm), washed (3×DMF, 3×DCM, 3× DMA), resuspended (DMA, 0.1 mL), incubated (30 min, room temperature, 600 rpm), and washed (1×DMA) prior to the first building block coupling. Library synthesis proceeded in eight steps: acylation, Fmoc removal, acylation with Fmoc-azido-proline, Fmoc removal or azide reduction, encoding oligonucleotide ligation, acylation, encoding oligonucleotide ligation, and global Fmoc removal/azide reduction.

Building block couplings. The first and second building block couplings consisted of acylation with an N-Fmoc-protected amino acid, while the third coupling comprised acylation with a carboxylic acid. In the first coupling, resin was resuspended (DMA, 0.15 mL) with building block/HOAt/DIC (6/6/8.5 μmol, respectively). Plates were covered with adhesive foil and incubated (1 h, 37° C., 600 rpm). Resin was washed (3×DMA, 3×DCM, 3 ×DMF), resuspended (DMF, 0.1 mL), and incubated (16 h, room temperature, 600 rpm). Resin was retrieved, washed (2×DMF), Fmoc was removed (20% piperidine in DMF, 1×5 min, 1×15 min, room temperature, 600 rpm), washed (3×DMF, 3×DCM, 3×DMA), resuspended (DMA, 0.1 mL), and incubated (30 min, room temperature, 600 rpm). The second and third building block couplings proceeded identically except that the second coupling step entailed simultaneous coupling of the cis - and trans-Fmoc-azido-proline isomers. Otherwise, resin was resuspended (DMA, 0.15 mL) with building block/Oxyma/TMP/DIC (12/12/12/15 μmol, respectively). Plates were covered with adhesive foil and incubated (3 h, 37° C., 600 rpm). Resin was washed (3×DMA, 3×DCM, 3×DMF), resuspended (DMF, 0.1 mL), and incubated (30 min, room temperature, 600 rpm). After the second building block coupling, resin was washed (2×DMF), Fmoc was removed from half the resin (20% piperidine in DMF, 1×5 min, 1×15 min, 600 rpm, room temperature) while azide was reduced on the other half of the resin (100 mM TCEP in DMF, 1 h, 37° C., 600 rpm), resin was washed (3×DMF, 3×DCM, 3×DMF, 3×1:1 DMF: BTPWB, 3×BTPWB), resuspended (BTPWB, 0.1 mL), and incubated (1 h, room temperature, 600 rpm). After the third building block coupling, an identical deprotection procedure was followed, except that all resin was subjected to both the Fmoc removal and azide reduction conditions as above.

Ligation of ≈22XX and ≈13XX encoding oligonucleotides. Resin was retrieved, washed (2×BTPWB, 1×BTPLB), resuspended (BTPLB, 0.1 mL), and incubated (30 min, room temperature, 600 rpm). An encoding oligonucleotide ligation mixture containing T4 DNA ligase (540 μg) in 2.25X BTPLB (20 mL) was prepared and aliquoted into all plate wells (0.1 mL) along with DI H₂O (26 μL). OP stocks of ˜22XX [±] (1.8 nmol, 12 μL) and ≈13XX [±] (1.8 nmol, 12 μL) were then added to the appropriate wells, the plate was sealed with adhesive foil, and incubated (4 h, room temperature, 600 rpm). Resin was washed (3×BTPWB, 3×1:1 DMF: BTPWB, 3×DMF), resuspended (DMF. 0.1 mL) and incubated (16 h, room temperature, 600 rpm). Resin was pooled into a reservoir, split into 192 wells (0.16 mg 160-μm resin, 63 nmol; 1.56 mg 10-μm resin, 450 nmol) of two fresh pre-wetted (3×DCM, 3×DMF) filtration microplates, and washed (3×DMF. 2×DMA) prior to coupling the third building block set.

Ligation of ≈24XX and ≈15XX encoding oligonucleotides. Ligation proceeded identically to above, except that OP stocks of ≈24XX [±] and ≈15XX [±] were used. Following ligation, pooling, and splitting, resin was washed (3×BTPWB, 3×1:1 DMF: BTPWB, 3×DMF), resuspended (BTPWB, 0.1 mL), and incubated (1 h, room temperature, 600 rpm) prior to the final ligation step.

Ligation of barcoding ˜26XX and ˜0B02 encoding oligonucleotides. An encoding oligonucleotide ligation mixture containing ˜0B02 [±] (360 nmol) and T4 DNA ligase (550 μg) in 2.25X BTPLB (20 mL) was prepared and aliquoted into plate wells (0.1 mL) along with DI H₂O (38 μL). OP stocks of ˜26XX [±] (1.8 nmol, 12 μL) were added to the appropriate wells, the plates were sealed with adhesive foil, and incubated (4 h, room temperature, 600 rpm). Resin was washed (3×BTPWB, 3×1:1 DMF: BTPWB, 3×DMF), resuspended (DMF, 0.1 mL), pooled, 160-μm QC beads were isolated by filtration (CellTrics 150 μm mesh, Sysmex Partec, Lincolnshire, IL), and stored in the dark (DMF, 4° C.).

Solid-phase DEL QC. An aliquot of 10-μm resin (0.06 mg) was transferred to a 1.5-mL tube, washed (BTPWB, 3×0.5 mL), and resuspended (BTPWB, 0.5 mL). The 10-μm bead concentration was determined by hemocytometer and diluted (1.2 beads/μL). The 160-μm QC resin was washed (10×BTPWB), resuspended (BTPWB, 1 mL), and an aliquot (3 mg) was separated for analysis.

Resin cleavage and MALDI-TOF MS analysis. Individual 160-μm beads (MeOH, 0.1 mL) were dried in vacuo (60° C.). A cleavage cocktail (90% TFA, 5% TIPS, 5% DCM, 10 μL) was added to dried single 160-μm bead samples, incubated (2 h, room temperature, 100 rpm), and dried in vacuo (60° C.). Compound was resuspended (50% ACN, 0.1% TFA in H₂O; 6 μL), a diluted (1:10) aliquot (1 μL) was co-spotted onto a MALDI-TOF MS target plate with HCCA matrix solution, dried, and analyzed via MALDI-TOF MS (Microflex, Bruker Daltonics, Inc., Billerica, MA) (Table 3).

DEL FACS screening for specific binding to DY-647-3×3 ILL: An aliquot of approximately 7.5×10⁶ DEL beads (100-fold greater than the number of unique compounds in the DEL) was filtered and then washed (i) once with N,N-dimethylformamide (DMF); (ii) once with 1:1 DMF: water; (iii) three times with Nanopure water; and (iv) three times with BTPWB. The beads were then equilibrated in BTPWB for 30 min at room temperature. The beads were counted using a hemocytometer under a microscope, diluted to a final concentration of 1×10⁶ beads/mL in 1× Blocking Buffer (BTPWB+200 nM BSA+200 nM bulk yeast tRNA+1% (v/v) Tween-20), and incubated overnight with shaking (300 rpm) at room temperature. The beads were then briefly vortexed and separated in two samples, a blank sample to establish a negative gate and establish thresholds (2.5×10⁶ beads) and a screening sample to screen for RNA binding (5×10⁶ beads).

Stocks of DY647-3×3 ILL (20 μM) and TAMRA-BP (200 μM) in Nanopure water were folded by heating at 60° C. for 5 min and cooled at room temperature for 5 min prior to use. To the screening sample, 5 μL of the TAMRA-BP stock was added to a final concentration of 200 nM, and the beads were briefly agitated. Then, 5 μL of the DY647-3×3 ILL stock was added to a final concentration of 20 nM. An equal volume of Nanopure water (10 μL) was added to the blank sample used for negative gating. The samples (blank and screening) were then incubated for 2 h with gentle shaking (300 rpm) at room temperature. They were filtered by using M1002 Mobicol Classic columns (Boca Scientific), washed three times with BTPWB, and resuspended in BTPWB at final concentration of 5×10⁶ beads/mL. Finally, the beads were filtered into a 5 mL round-bottom tube through a cell-strainer cap and subjected to FACS on the BD FACSAria3 (BD Biosciences). At least 8×10⁵ beads were analyzed for sorting to cover the library diversity with a redundancy of 10. Beads showing an increase of DY647 fluorescence without any increase of TAMRA fluorescence were isolated and pooled together in a 1.6 mL tube. Beads were washed five times with water, with centrifugation at 5000 rpm for 5 min and careful removal of the supernatant between washes.

qPCR analysis. qPCR analysis proceeded as previously described with modifications to the 160-μm bead DNA amplification procedure. qPCR matrix for 10-μm beads contained Taq DNA Polymerase (0.05 U/μL), oligonucleotide primers 5′-GCCGCCGCCTTCGTCCTTCTCAGCGAC-3′ SEQ ID NO: 1 and 5′-/5AmMC6/GTGGCACAACAACTGGCGGGCAAAC-3′ SEQ ID NO:2 (0.3 μM each), SYBR Green (0.2×, Life Technologies), and GC-PCR buffer (1×). Diluted (1.2 beads/μL) and undiluted (100 beads/μL) 10-μm library beads (BTPWB, 1 μL) were added to separate amplification wells containing qPCR matrix (20 μL, 33 and 10 replicates, respectively). The supernatant for each resin sample (1 μL) was added to separate amplification wells (20 μL, 2 replicates). Template standard solutions (1 fmol, 100 amol, 10 amol, 1 amol, 100 zmol, 10 zmol, 1 zmol, 100 ymol, and 10 ymol, each in 1 μL BTPWB) were added to separate amplification reactions (20 μL). Reactions were thermally cycled (96° C., 10 s; [95° C., 8s; 72° C., 24 s]×32 cycles; 72° C., 2 min); with fluorescence monitoring (channel 1, CFX96 Real-Time System, Bio-Rad) and quantitated (CFX Manager, Version 3.1, Bio-Rad, baseline subtracted). The number of amplifiable tags per bead was calculated by dividing the qPCR result by the number of beads per well (confirmed using a stereo zoom microscope).

Amplification proceeded identically for 160-μm beads, except that individual beads were added to separate wells for amplification, the qPCR matrix contained 0.6 μM 5′-/5AmMC6/GTGGCACAACAACTGGCGGGCAAAC-3′ SEQ ID NO:3, and the reaction thermal cycling was modified (96° C., 10 s; [95° C., 8s; 72° C., 120 s] x 29 cycles; 72° C., 2 min).

Amplification and sequencing. Single 160-μm resin beads (33) were retrieved via pipet from PCR plate wells and deposited into a 96-well microplate (MeOH, 0.1 mL). Each 160-μm library bead PCR sample (5 μL) was purified by native PAGE (6%, 1×TBE, 12 W, 30 min). Gel slices containing 182-nt DNA products were excised and eluted prior to amplification (C&S, 0.1 mL, 16 h, room temperature, 8 rpm). PCR matrix contained Taq DNA Polymerase (0.05 U/μL), oligonucleotide primers 5′-GTTTTCCCAGTCACGAC-3′ SEQ ID NO:4 (0.3 μM) and 5′-GTGGCACAACAACTG-3′SEQ ID NO:5 (0.28 μM) and 5′-CGCCAGGGTTTTCCCAGTCACGACCAACCACCCAAACCACAAA CCCAAACCCCAAACCCAACACACAACAACAGCCGCCGCCTTCGTCCTTCTCAGCG AC-3′ SEQ ID NO:6 (0.02 UM, FOX primer), and GC-PCR buffer (1×). PAGE-purified PCR products (2 μL) were added to separate amplification reactions (50 μL) and thermally cycled (95° C., 2 min; [95° C., 20 s; 52° C., 15 s; 72° C., 20 s] x 34 cycles; 72° C., 2 min). PCR products were purified (QIAquick PCR purification kit, QIAGEN, Valencia, CA) and sequenced using the primer 5′—CGCCAGGGTTTTCCCAGTCACGAC-3′ SEQ ID NO:7. Sequencing reads were trimmed to remove all called bases prior to the opening primer sequence (5′-GCCGCCCAGTCCTGCTCGCTTCGCTAC-3′ SEQ ID NO:8). Sequences were aligned to a degenerate reference sequence (5′-ATGGNNNNNNNNTCANNNNNNNNGTTNNNNNNNNCTANNNNNNNNTTCNNNNN NNNCGCNNNNNNNNGCCTCCCAAACNNNNNNNNGTT-3′ SEQ ID NO:9) and the encoding regions (5′—NNNNNNNN-3′ SEQ ID NO:10) were matched to the building block alpha-numeric identifier lookup table to assign the synthesis history for each compound.

Hit amplification and preparation for NGS. Samples for NGS analysis were prepared as previously described. (5, 6) The qPCR matrix was prepared containing Taq DNA polymerase (0.05 U/μL), oligonucleotide primers 5′-GCCGCCGCCTTCGTCCTTCTCAGCGAC-3′ SEQ ID NO:11 (0.3 μM) and 5′-/5AmMC6/GTGGCACAACAACTGGCGGGCAAAC-3′ SEQ ID NO: 12 (0.6 UM), SYBR Green (0.2×, Life Technologies), and GC-PCR buffer (1×). qPCR matrix was added to 0.2 mL tubes (40 μL). Template standard solutions (100 amol, 10 amol, 1 amol, 100 zmol, 10 zmol, 1 zmol, 100 ymol, and 10 ymol, each in 1 μL BTPWB) were added to separate amplification reactions (40 μL). Hit beads (n=˜ 130; 0.017% hit rate) were washed (2×200 μL BTPWB, 1×200 μL PCR buffer) and resuspended in qPCR matrix (40 μL). Reactions were thermally cycled (96° C., 10 s; [95° C., 8s; 72° C., 120 s] x 29 cycles; 72° C., 2 min). Samples were centrifuged (5 s, 2,000 rcf), then the supernatant was collected and diluted (10,000-fold). PCR matrix contained Taq DNA polymerase (0.05 U/μL), oligonucleotide primer 5′-CCTCTCTATGGGCAGTCGGTGATGCCGCCGCCTTCGTCCTTCTCAGCGAC-3′ SEQ ID NO: 13 (0.3 μM), sequencing barcode oligonucleotide primer 5′-CCATCTCATCCCTGCGTGTCTCCGACTCAGNNNNNNNNNNGATGCCGCCCAGTCC

TGCTCGCTTCGCTAC-3′ SEQ ID NO: 14 (0.3 μM), SYBR Green (0.2×, Life Technologies) DMSO (6%), betaine (1 M), MgCl₂ (1 mM) and PCR buffer (1×). Amplicon supernatant (2 μL) and a corresponding sequencing barcode oligonucleotide primer (5′-CCATCTCATCCCTGCGTGTCTCCG ACTCAGNNNNNNNNNNGATGCCGCCCAGTCCTGCTCGCTTCGCTAC-3′ SEQ ID NO: 15. (0.3 μM) were added to separate amplification wells (40 μL). Reactions were thermally cycled ([95° C., 8 s; 70° C., 24 s; 72° C., 16 s]×20 cycles; 72° C., 2 min). Amplicons were pooled and purified (25 μL) by native PAGE (6%, 1×TBE, 8 W, 30 min) with SYBR Gold staining (Life Technologies, Inc.). Gel bands containing the 210-bp DNA products were excised, eluted (DI H2O, 0.1 mL, 16 h, room temperature, 8 rpm), and used for standard DNA sequencing library preparation and analysis (Ion Proton, Life Technologies, Inc.).

NGS data processing. Sequence trimming, pattern matching, and UMI aggregation proceeded as previously described. (5, 6) Sequences were ranked by UMI mean string distance and UMI count. Beads with mean string distance <5 and UMI count <10 were not considered. Compound replicates were calculated as the sum of remaining sequences having identical structure-encoding regions but distinct bead-specific barcodes. (7)

Cheminformatic analysis. All in silico combinatorial library and hit cheminformatic analysis was performed using open-access software (Data Warrior v 4.7.2). (8) Hits were clustered by chemical similarity (T >0.75).

Synthesis of the hit compounds: Hit compounds emerging from the FACS screen were synthesized in parallel on Rink Amide Polystyrene resin (50 mg, 27.5 μmol). The resin was swollen in mL DMF for 10 min, filtered, and deprotected with 1 mL of 20% piperidine in DMF twice for 5 min each. The resin was washed (5×DMF) followed by bromoacetylation with 1 mL of a cocktail containing 20% DIC and 80% 1.2M bromoacetic acid in DMF for 30 min. After washing (5×DMF), bromide displacement was accomplished by treatment with 1 mL of 1 M propargylamine dissolved in DMF for 1 h, followed by additional washing (5×DMF).

The first building block (Fmoc-Xaa-OH) coupling was performed in DMF (160 mM, 1 mL) in the presence of HOAt (160 mM) and DIC (114 mM) for 1 h at 37° C. After washing (5×DMF), the resin was treated with 1 mL of 20% piperidine in DMF twice for 5 min each, then washed again (5×DMF). Fmoc-protected azidoproline (3S or 3R) was coupled as described for the previous coupling. Then, resin was either treated with A) 20% piperidine in DMF twice for 5 min each or B) TCEP (100 mM, 80% DMF and 20% water) for 1 h at 37° C., depending on the deconvoluted regiochemistry, then washed (A: 5×DMF; B: 2×H₂O, 5×DMF).

The last building block (R—COOH) was coupled in DMF (320 mM, 1 mL) in the presence of Oxyma (320 mM), DIC (200 mM) and TMP (160 mM) for 1 h at 37° C. After washing (5×DMF), the resin was either treated with A) 20% piperidine in DMF twice for 5 min each or B) TCEP (100 mM, 80% DMF and 20% water) for 1 h at 37° C., depending on the deconvoluted regiochemistry, then washed (A: 5×DMF, 5×DCM; B: 2×H₂O, 5×DMF, 5×DCM). The compounds were cleaved from the resin with a cocktail containing 95% TFA. 2.5% water, and 2.5% TIPS. The cleavage cocktail was evaporated under a stream of nitrogen to afford the crude compounds. Compounds were solubilized and purified by preparative HPLC as described above. Fractions containing the expected mass were pooled and evaporated to dryness under vacuum prior to full characterization.

Compound 1 was synthesized using the general procedure above following deconvoluted synthesis history: cas #111524-95-9, cas #263847 Aug. 1, piperidine, cas #244126-64-5, TCEP. After HPLC purification 5.8 μmol of 1 was obtained (21% yield). (C₂₈H₂₆F₃N₅O₄S) calculated [M+H]⁺: 586.1731 Da; found: 586.1646 Da (14 ppm); tR: 36.0 min.

Compound 2 was synthesized using the general procedure above following deconvoluted synthesis history: cas #111524-95-9, cas #702679-55-8, piperidine, cas #244126-64-5, TCEP. After HPLC purification 14.9 μmol of 1 was obtained (54% yield). (C₂₈H₂₆F₃N₅O₄S) calculated [M+H]⁺: 586.1731 Da; found: 586.1566 Da (28 ppm); tR: 35.9 min.

Compound 3 was synthesized using the general procedure above following deconvoluted synthesis history: cas #145484-45-3, cas #263847 Aug. 1, piperidine, cas #10406-05-0, TCEP. After HPLC purification 3.8 μmol of 1 was obtained (14% yield). (C₂₉H₃₆CIN₇O₅) calculated [M+H]⁺: 598.2539 Da; found: 598.2488 Da (8 ppm); tR: 34.8 min.

Compound 4 was synthesized using the general procedure above following deconvoluted synthesis history: cas #145484-45-3, cas #702679-55-8, piperidine, cas #10406-05-0, TCEP. After HPLC purification 11.4 μmol of 1 was obtained (41% yield). (C₂₉H₃₆CIN₇O₅) calculated [M+H]⁺: 598.2539 Da; found: 598.2350 Da (32 ppm); tR: 34.0 min.

Compound 5 was synthesized using the general procedure above following deconvoluted synthesis history: cas #136552 Jun. 2, cas #263847 Aug. 1, piperidine, cas #73728-40-2, TCEP. After HPLC purification 16.0 μmol of 1 was obtained (62% yield). (C₂₆H₃₂N₆O₆) calculated [M+H]⁺: 525.2456 Da; found: 525.2299 Da (30 ppm); tR: 17.9 min. Compound 6 was synthesized using the general procedure above following deconvoluted synthesis history: cas #136552 Jun. 2, cas #702679-55-8, piperidine, cas #73728-40-2, TCEP. After HPLC purification 17.7 μmol of 1 was obtained (64% yield). (C₂₆H₃₂N₆O₆) calculated [M+H]⁺: 525.2456 Da; found: 525.2225 Da (44 ppm); tR: 17.9 min. Compound 7 was synthesized using the general procedure above following deconvoluted synthesis history: cas #401933-16-2, cas #263847 Aug. 1, TCEP, cas #87392-05-0, piperidine. After HPLC purification 3.2 μmol of 1 was obtained (12% yield). (C₂₅H₃₀N₆O₅) calculated [M+H]⁺: 495.2228 Da; found: 495.2228 Da (25 ppm); tR: 22.8 min.

Compound 8 was synthesized using the general procedure above following deconvoluted synthesis history: cas #401933-16-2, cas #702679-55-8, TCEP, cas #87392-05-0, piperidine. After HPLC purification 5.7 μmol of 1 was obtained (21% yield). (C₂₅H₃₀N₆O₅) calculated [M+H]⁺: 495.2228 Da; found: 495.2242 Da (22 ppm); tR: 22.2 min.

Compound 9 was synthesized using the general procedure above following deconvoluted synthesis history: cas #353245-98-4, cas #263847 Aug. 1, TCEP, cas #78754-94-6, piperidine. After HPLC purification 6.5 μmol of 1 was obtained (24% yield). (C₃₂H₃₄Ng06) calculated [M+H]⁺: 627.2639 Da; found: 627.2639 Da (5.5 ppm); tR: 31.3 min.

Compound 10 was synthesized using the general procedure above following deconvoluted synthesis history: cas #353245-98-4, cas #702679-55-8, TCEP, cas #78754-94-6, piperidine. After HPLC purification 8.1 μmol of 1 was obtained (29% yield). (C₃₂H₃₄N₈O₆) calculated [M+H]⁺: 627.2639 Da; found: 627.2833 Da (25 ppm); tR: 29.9 min.

### IN VITRO METHODS

2DCS: Preparation of small molecule microarrays and RNA selection: Preparation of azide functionalized glass slides: Briefly, a 2 mL aliquot of 1% (w/v) molten agarose solution (prepared in Nanopure water) was applied to a silane-coated glass slide, and the agarose was allowed to dry overnight. The slides were then immersed in 20 mM NaIO4 and gently shaken at room temperature for 30 min. The slides were washed with Nanopure water twice for 15 min each and then immersed in 10% (v/v) ethylene glycol and shaken at room temperature for 1.5 h. After washing the slide twice in Nanopure water twice for 15 min each, they were immersed in 20 mM azido propylamine prepared in 0.1 M NaHCO₃ and shaken at room temperature overnight. The slides were then reduced by immersing the slides in a solution of 100 mg NaBH₃CN in 10 mL ethanol and 40 mL 1×PBS for 30 min at room temperature. The slides were then washed twice with Nanopure water for 15 min each and dried completely on the benchtop.

Conjugation of compounds to azido-functionalized microarrays: Compounds at varying concentrations (0.5 μL in DMSO) were combined with an equal volume of “Click Reaction Mixture” comprised of CuSO₄ (10 mM, 0.1 μL), THPTA (50 mM, 0.1 μL), sodium ascorbate (250 mM, 0.1 μL) and phosphate buffer (0.2 μL, 20 mM sodium phosphate, pH 7.5). This mixture was then spotted onto the array surface, and the array was incubated at 37° C. for 3 h in a humidity chamber. After 3 h, the slides were washed with Nanopure water twice and allowed to dry completely on the benchtop.

2DCS selection: The 3×3 ILL was 5′-end labeled with ³²P and purified as previously described. (9) The RNA library was folded in 1× Binding Buffer (BB1; 8 mM Na2HPO4, pH 7.0, 185 mM NaCl, 1 mM EDTA) by heating at 60° C. for 10 min followed by cooling to room temperature on the bench top. All competitor oligos (C1-C8), each in an amount equivalent to the number of total compound delivered to the array surface, were folded separately in 1× AB1 as described for 3×3 ILL. The folded oligos were mixed together with 5′-³²P labeled 3×3 ILL followed by addition of MgCl₂ (1 mM) and bovine serum albumin (BSA, 120 μg/mL) in a total volume of 600 μL. The array surface was preequilibrated with 1× BB2 (1× BB1 supplemented with 1 mM MgCl₂ and 120 μg/mL BSA) for 5 min, after which the excess buffer was removed. The mixture of 3×3 ILL and competitor oligonucleotides was then applied to the surface, and the array was incubated for 20 min at room temperature. The glass slide was then washed with 1× AB2 three times and dried for 1 h. The array was imaged by using Molecular Dynamics Typhoon variable mode phosphorimager.

Reverse transcription and PCR amplification to install barcodes to encode each compound were performed as previously described. (10) The DNA thus obtained was purified using native 8% polyacrylamide gel electrophoresis (PAGE) and its purity confirmed via bioanalyzer. The bar-coded samples were mixed in equimolar amounts and sequenced using an Ion Proton deep sequencer. The sequencing data obtained were statistically analyzed for enrichment according to previously reported protocol, affording Z_{obs} for each small molecule-RNA interaction. (10)

Binding affinity measurements: Binding affinities were measured by microscale thermophoresis (MST), performed on a Monolith NT.115 system (NanoTemper Technologies) with Cy5-labeled RNAs. These RNAs include: the 5′GAG/3′CCC SEQ ID NO: 16 loop at pri-miR-27a's Drosha site (Sequence:/5′-Cy5/rCrUrGrArGrGrUrGrArArArCrArUrCrCrCrArG SEQ ID NO: 17; Dharmacon), a related internal loop not selected by 9. 5′CAG/3′GCC SEQ ID NO: 17 loop (Sequence:/5′-Cy5/rCrUrCrArGrGrUrGrArArArCrArUrCrCrGrArG SEQ ID NO: 16; Dharmacon), and an RNA in which the loop is mutated to a base pair (Sequence:/5-Cy5/rCrUrGrArGrGrUrGrArArArCrArUrCrUrCrArG SEQ ID NO: 16; Dharmacon).

Briefly, Cy5-labeled RNA (10 nM) was prepared in 1× Binding Buffer and folded by heating at 60° C. for 5 min and then slowly cooling to room temperature. After cooling, Tween-20 was added to a final concentration of 0.1% (v/v). Compound solutions were prepared separately in 20 μL of 1× Binding Buffer at a final concentration of 5 μM (1% (v/v) DMSO), followed by 1:1 serial dilutions in 1× Binding Buffer. RNA and compound solutions were then mixed 1:1 by volume to a total of 20 μL.

Samples were incubated for 20 min at room temperature and then loaded into premium capillaries (NanoTemper Tech, Cat #MO-K025). The following parameters were used for MST measurements: 5-20% LED power (adjusted to keep fluorescence intensity between 2000 and 8000), 80% MST power, Laser-On time=30 s, Laser-Off time=25 s. The resulting data were analyzed by calculating the change in thermophoresis as a function of compound concentration and fitted by Equation 1, a one site binding model, in NanoTemper Tech's MST analysis software to yield the dissociation constant (K_{d}):

$$f ( c ) = unbound + \frac{bound - unbound}{2} * ( F + c + {K}_{d} - \sqrt{( {( F + c + {K}_{d} )}^{2} - 4 * F * c ) )} \qquad ( Eq 1 )$$

Where F is the concentration of fluorescently labeled RNA; unbound and bound refer to the thermophoresis signal at completely unbound and bound state of RNA, respectively; c is the concentration of the compound; f (c) is the thermophoresis signal at compound concentration of c; and K_{d} is the dissociation constant.

For competitive binding assays, both WT and mutant pri-miR-27a RNAs were transcribed as previously described (see Table S6 for sequences). (11) The RNA was purified by gel electrophoresis and folded in 1× Binding Buffer at final concentration of 750 nM by heating at 60° C. for 5 min and then slowly cooling to room temperature. This RNA was aliquoted (10 μL) and diluted with an equal volume of 1× Binding Buffer. To each pri-miR-27a sample was added 10 μL of folded Cy5-labeled 5′GAG/3′CCC SEQ ID NO: 18 loop displayed in pri-miR-27a's Drosha site (folded as described above; 5 nM final concentration). To each tube was then added 10 μL of 750 nM of 9 (final concentration of 100 nM), prepared in1× Binding Buffer containing 0.05% (v/v) Tween-20. The samples were incubated at room temperature for 20 min, followed by thermophoresis analysis as described above. The resulting data were analyzed by SigmaPlot and fitted by Equation 2 to yield the competitive dissociation constant (K_{d} competitive).

$$f ( c ) = a * ( \frac{1}{2 [ F ]} ) * {( {K}_{c} + ( \frac{{K}_{c}}{{K}_{d}} ) * c + 2 [ F ] - {( {K}_{c} + ( \frac{{K}_{c}}{{K}_{d}} ) * c + 2 [ F ] )}^{2} - {4 [ F ]}^{2} )}^{0.5} + A \qquad ( eq 2 )$$

where F is the concentration of fluorescence-labeled RNA; unbound and bound refers to the thermophoresis signal at completely unbound and bound state of RNA, respectively; c is the concentration of the compound; f (c) is the thermophoresis signal at compound concentration of c; K_{d} is the dissociation constant. K_{c} is the competitive dissociation constant.

### Cellular Methods

Cell lines. Compounds were tested in MDA-MB-231 TNBC cells (HTB-26, ATCC), LNCaP metastatic prostate cancer cells (gifted from Junli Luo lab), MCF-7 (HTB-22, ATCC) breast cancer cells, HeLa cervical adenocarcinoma cells (CCL-2, ATCC), and MCF-10a, a model of healthy breast epithelial cells (CRL-10317, ATCC).

Cell culture and compound treatment. All cells were maintained at 37° C. with 5% CO₂. MDA-MB-231 and LNCaP cells were cultured in RPMI 1640 medium with L-glutamine & 25 mM HEPES (Corning) supplemented with 10% (v/v) fetal bovine serum (FBS; Sigma) and 1× (v/v) Antibiotic-Antimycotic solution (Corning). MCF-7 and HeLa cells were cultured in DMEM medium with 4.5 g/L glucose (Corning), supplemented with 10% FBS, 1× (v/v) Glutagro (Corning), and 1× (v/v) Antibiotic-Antimycotic solution. MCF10a cells were cultured in DMEM/F_{12 50/50} with L-glutamine & 15 mM HEPES (Corning), supplemented with 10% FBS, 20 ng/mL human epidermal growth factor (Pepro Tech Inc.), 0.5 mg/mL hydrocortisone (Pfaltz & Bauer), 100 ng/mL cholera toxin (Sigma-Aldrich), 10 μg/mL insulin (Sigma-Aldrich), and 1× (v/v) Antibiotic-Antimycotic solution.

MCF-10a cells were transfected with plasmids to express pri-miR-27a and mutant pri-miR-27a in 12- or 24-well plates with Lipofectamine 2000 per the manufacturer's protocol. For treatment of compounds, stocks were diluted in growth medium and added to cells for 48 h. The miRCURY miR-27a LNA inhibitor (QIAGEN, Cat #339121) was used as a positive control by diluting in growth medium to a final concentration of 1 nM. The miRCURY LNA Negative Control (QIAGEN, Cat #YI00199007-DDA) was used as a negative control, which has also diluted in growth medium to a final concentration of 1 nM.

The plasmids encoding pri-miR-27a and mutant pri-miR-27a were custom synthesized by GenScript. Wild type miR-27a hairpin plasmid (Cat #SC1692) was produced via VectorArk Vector MR04 with cloning direction consistent to promoter. Mutant miR-27 hairpin plasmid (Cat #SC1441) was generated by mutagenesis on the wild type plasmid.

Analysis of mRNA abundance: Each cell line was grown as a monolayer in 12- or 24-well plates and treated as described in “Cell culture and compound treatment”. After 48 h, the cells were lysed, and total RNA was harvested using a Zymo Quick RNA Miniprep Kit per the manufacturer's protocol. To measure the abundance of mature miRNAs, approximately 250 ng of total RNA was reverse transcribed using the High Flex Buffer provided in a miScript II RT Kit (Qiagen) per the manufacturer's protocol (10 μL total reaction volume). For pri-miRNAs and mRNA, approximately 300 ng of total RNA was reverse transcribed using a qScript cDNA synthesis kit (10 μL total reaction volume, Quanta BioSciences). For all types of RNAs, 2 μL of the RT reaction was used for qPCR using SYBR Green Master Mix and a QuantStudio™ 5 Real-Time PCR System. Relative abundance of mature miRNAs was calculated by normalizing to RNU6, and relative abundance for pri-miRNAs and mRNAs was calculated by normalizing to 18S ribosomal RNA, using the AAC, method. (12)

Western Blotting: MDA-MB-231 cells were grown in 6-well plates to ˜80% confluency in complete growth medium and then incubated with 9 at the indicated concentrations for 48 h. Total protein was extracted using M-PER Mammalian Protein Extraction Reagent (Pierce Biotechnology) using the manufacturer's protocol and quantified using a Micro BCA Protein Assay Kit (Pierce Biotechnology). Approximately 50 μg of total protein was separated on a 10% SDS-polyacrylamide gel, and then transferred to a PVDF membrane. The membrane was washed with 1× Tris-buffered saline (TBS) and then blocked in 1×TBST (1×TBS containing 0.1% (v/v) Tween-20) containing 5% (w/v) milk for 1 h at room temperature. After washing with 1×TBST, the membrane was incubated with a 1:1000 dilution of rabbit anti-PDCD4 (Cell Signaling Technology: D29C₆), a 1:2000 dilution of rabbit anti-ZBTB10 (catalog number: ab117786; Abcam), or a 1:2000 of rabbit anti-PP4C (catalog number: ab227267; Abcam) in 1×TBST containing 5% milk overnight at 4° C. The membrane was then washed with 1×TBST and incubated with 1:5000 anti-rabbit IgG horseradish-peroxidase secondary antibody conjugate (catalog number: 7076; Cell Signaling Technology) in 1×TBS for 2 h at room temperature. The membrane was again washed with 1×TBST, and protein expression was quantified using SuperSignal West Pico Chemiluminescent Substrate (Pierce Biotechnology) per the manufacturer's protocol.

To quantify b-actin expression, used for normalization, the membrane was stripped using 1× Stripping Buffer (200 mM glycine, pH 2.2 and 0.1% SDS) followed by washing in 1×TBST. The membrane was blocked and probed for b-actin as described above using a 1:5000 dilution of mouse anti-b-actin antibody (8H10D10; catalog number: 3700S; Cell Signaling Technology) at room temperature for 2 h and 1:5000 anti-mouse IgG horseradish-peroxidase secondary antibody conjugate (catalog number: 7074; Cell Signaling Technology) in 1×TBS for 2 h at room temperature. The fold change of the target protein expression (PP4C or PDCD4) was calculated by normalizing its band intensity to b-actin band intensity using ImageJ.

Migration assay: MDA-MB-231 cells were grown in 60 mm diameter dishes and treated as described in “Cell culture and compound treatment” for 12 h. The medium was then removed and replaced with growth medium lacking FBS but with the same concentration of compound. After 12 h of serum starvation, the cells were detached and seeded to ThinCert™ (GBO) 24-well hanging inserts with 8 μm pores (˜5×10⁴ cells per insert). Fresh growth medium with FBS was dispended in a 24-well plate (600 μL each well), and the ThinCert™ hanging inserts containing cells were then placed into the wells. After incubating for 24 h, the growth medium in the inserts was removed, and the remaining cells were washed twice with 1×DPBS. Cells were fixed by addition of 3% (w/v) paraformaldehyde (PFA) prepared in 1×DPBS at room temperature for 20 min. The PFA solution was removed, and cells were washed twice with 1×PBS. The cells were then stained with Crystal Violet (10 mg/mL; 4:1 H₂O: MeOH) at room temperature for 20 min. A cotton swab was used to gently remove non-migrating cells from the top side of the filter. The remaining cells were then imaged by microscopy and counted for quantification (3 fields of view per sample).

### Proteomics

Global proteomics profiling using LC-MS/MS: Treated cell pellets were resuspended in 1×PBS, lysed by sonication, and protein concentration was determined by using a Bradford assay (Bio-Rad). Samples (30 μg) were denatured in 6 M urea in 50 mM NH₄HCO₃, pH 8, reduced for 30 min with 10 mM TCEP, and alkylated for 30 min in the dark with 25 mM iodoacetamide. Samples were diluted to 2 M urea with 50 mM NH₄HCO₃, pH 8, and the proteins were digested with trypsin (1 μL of 0.5 μg/μL) in the presence of 1 mM CaCl₂) for 12 h at 37° C. The samples were acidified by adding acetic acid to a final concentration of 5% (v/v). After desalting the samples over a self-packed C₁₈ spin column, they were dried and analyzed by LC-MS/MS (see below). The resulting MS data were processed with MaxQuant as described below.

LC-MS/MS analysis: Peptides were dissolved in water containing 0.1% formic acid (FA) and analyzed using an EASY-nLC 1200 nano-UHPLC connected to a Q Exactive HF-X Quadrupole-Orbitrap mass spectrometer (Thermo Scientific). A 50 cm long, 75 μm i.d. chromatography column packed with ReproSil-Pur 120 C18-AQ 2.4 μm beads (Dr. Maisch GmbH) and capped by a 5 μm tip was used. Water with 0.1% FA in water (Buffer A) and 90% acetonitrile (MeCN): 10% water with 0.1% FA (Buffer B) were used as liquid chromatography solvents. A flow rate of 300 nL/min over a 240 min linear gradient (5-35% Buffer B) at 65° C. was used to clute peptides into the mass spectrometer. Data-dependent acquisition (top-20, NCE 28, R=7,500) after full MS scan (R=60,000, m/z 400-1,300) with a dynamic exclusion of 10 seconds was performed. Peptide match to prefer and isotope exclusion were selected and enabled.

MaxQuant analysis: The MaxQuant software (13) (V1.6.1.0) was used to analyzed the MS data and searched against the human proteome (Uniprot) and a common list of contaminants (included in MaxQuant). Peptide search tolerance was set to 20 ppm for the first search and 10 ppm for the main search while 0.02 Da was used for the fragment mass tolerance. The false discovery rate (FDR) for peptides, proteins and sites identification was set to 1%. Peptide length was set to at least 6 amino acids and peptide re-quantification was enabled. Label-free quantification (MaxLFQ) and “match between runs” were activated. Number of peptides per protein was set to ≥2. Searched modifications were methionine oxidation (variable modification) and carbamidomethylation of cysteines (fixed modification).

TargetScan analysis: TargetScanHuman v7.2 was used to predict downstream protein targets of miR-27a-3p (n=1421), miR-23a-3p (n=1342) and miR-24-3p (n=761) containing conserved sites, all proteins with a context score≤0 were included to the analysis per TargetScan recommendation. Approximately 15% of miR-27a-3p targets (220/1421), ˜18% of miR-23a-3p targets (247/1342), and ˜16% of miR-24-3p targets (122/761) were detectable in the global proteomics analysis. Cumulative distribution plots of the fold change of proteins in 9-treated vs. vehicle-treated samples indicated a significant upregulation of only miR-27a-3p targets (red), while no significant change was observed with miR-23a-3p targets (green) and miR-24-3p (blue), relative to the cumulative distribution of all proteins (black) (FIG. 4B). Targets of miR-23a-3p and miR-24-3p were used as comparison as the two miRNAs are expressed at similar levels as miR-27a-3p in MDA-MB-231 cells and are located in the same cluster. P values between distributions were calculated using a two-tailed Kolmogorov-Smirnov test.

TABLES
Table 1. Sequences of oligonucleotides overhangs,
and PCR primers used to construct the DEL.

| Overhang | | |
|---|---|---|
| ID | Overhang [+] | Overhang [−] |
| ≈X1XX[+] | /5Phos/ATGG | /5Phos/TGA |
| ≈X2XX[+] | /5Phos/TCA | /5Phos/AAC |
| ≈X3XX[+] | /5Phos/GTT | /5Phos/TAG |
| ≈X4XX[+] | /5Phos/CTA | /5Phos/GAA |
| ≈X5XX[+] | /5Phos/TTC | /5Phos/GCG |
| ≈X6XX[+] | /5Phos/CGC | /5Phos/AGGC |
| PCR Primer | | |
| ID | PCR Primer [+] | PCR Primer [−] |
| ≈0002 | /5Phos/GCCGCCGCC | 5'/5Phos/CCATG |
| | TTCGTCCTTCTCAGCG | TCGCTGAGAAGGAC |
| | AC SEQ ID NO: 18 | GAAGGCGGCGGCGG |
| | | SEQ ID NO: 19 |
| ≈0B02 | /5Phos/GCCTCCCAA | GTTTGGG |
| | ACNNNNNNNNGTTTGC | SEQ ID NO: 21 |
| | CCGCCAGTTGTTGTGC | |
| | CAC SEQ ID NO:20 | |

Phos indicates phosphate; N indicates any nucleotide (A, G, C, or T)

TABLE 2
DNA tags used to decode DEL building blocks.

| | | SEQ | | SEQ | | | SEQ | | SEQ |
|---|---|---|---|---|---|---|---|---|---|
| Code 1 ID | Code 1 [+] | ID: | Code 1 [−] | ID: | Code 2 ID | Code 2[+] | ID: | Code 2 [−] | ID: |
| ≈1X17 | ACAAGAAA | 22 | TTTCTTGT | 63 | ≈2X01 | CCTCCTAA | 105 | TTAGGAGG | 165 |
| ≈1X18 | ACAAGGCT | 23 | AGCCTTGT | 64 | ≈2X02 | AACCTCAA | 106 | TTGAGGTT | 166 |
| ≈1X19 | ACAGGGTA | 24 | TACCCTGT | 65 | ≈2X03 | AATCCCAT | 107 | ATGGGATT | 167 |
| ≈1X20 | ACGAAAGA | 25 | TCTTTCGT | 66 | ≈2X04 | AACCCTAC | 108 | GTAGGGTT | 168 |
| ≈1X21 | ACGAGATT | 26 | AATCTCGT | 67 | ≈2X05 | ATCCTCTC | 109 | GAGAGGAT | 169 |
| ≈1X22 | ACGAGGGC | 27 | GCCCTCGT | 68 | ≈2X06 | CATTTCAA | 110 | TTGAAATG | 170 |
| ≈1X23 | ACGGAATC | 28 | GATTCCGT | 69 | ≈2X07 | CGCCTTCA | 111 | TGAAGGCG | 171 |
| ≈1X24 | ACGGGAAG | 29 | CTTCCCGT | 70 | ≈2X08 | CGTTCCTG | 112 | CAGGAACG | 172 |
| ≈1X25 | AGAAGACC | 30 | GGTCTTCT | 71 | ≈2X09 | TTCTTCAT | 113 | ATGAAGAA | 173 |
| ≈1X26 | AGGAAGGG | 31' | CCCTTCCT | 72 | ≈2X10 | TCCTCTTA | 114 | TAAGAGGA | 174 |
| ≈1X27 | AGGGAAAT | 32 | ATTTCCCT | 73 | ≈2X11 | AACCTTCG | 115 | CGAAGGTT | 175 |
| ≈1X28 | ATAAGGGA | 33 | TCCCTTAT | 74 | ≈2X12 | AACTCCCG | 116 | CGGGAGTT | 176 |
| ≈1X29 | ATAGAGCC | 34 | GGCTCTAT | 75 | ≈2X13 | AACTCTTT | 117 | AAAGAGTT | 177 |
| ≈1X30 | CAAAGACT | 35 | AGTCTTTG | 76 | ≈2X14 | AATCCTCA | 118 | TGAGGATT | 178 |
| ≈1X31 | CAAAGGAC | 36 | GTCCTTTG | 77 | ≈2X15 | AATCTCCC | 119 | GGGAGATT | 179 |
| ≈1X32 | CAAGAAGA | 37 | TCTTCTTG | 78 | ≈2X16 | AATCTTGT | 120 | ACAAGATT | 180 |
| ≈1X33 | CAAGAGTC | 38 | GACTCTTG | 79 | ≈2X17 | AATTCCGA | 121 | TCGGAATT | 181 |
| ≈1X34 | CAGAAGGA | 39 | TCCTTCTG | 80 | ≈2X18 | ACCCTCCT | 122 | AGGAGGGT | 182 |
| ≈1X35 | CAGAGAAA | 40 | TTTCTCTG | 81 | ≈2X19 | ACCCTTGA | 123 | TCAAGGGT | 183 |
| ≈1X36 | CAGGGACG | 41 | CGTCCCTG | 82 | ≈2X20 | ACCTCCAA | 124 | TTGGAGGT | 184 |
| ≈1X37 | CCGAAACT | 42 | AGTTTCGG | 83 | ≈2X21 | ACCTCTCC | 125 | GGAGAGGT | 185 |
| ≈1X38 | CCGAGGAG | 43 | CTCCTCGG | 84 | =2X22 | ACCTTCGC | 126 | GCGAAGGT | 186 |
| ≈1X39 | CCGGAGGG | 44 | CCCTCCGG | 85 | ≈2X23 | ACTCCCGC | 127 | GCGGGAGT | 187 |
| ≈1X40 | CGAGAACC | 45 | GGTTCTCG | 86 | ≈2X24 | ACTCCTTT | 128 | AAAGGAGT | 188 |
| ≈1X41 | CGAGGAGG | 46 | CCTCCTCG | 87 | ≈2X37 | CACCTCGC | 129 | GCGAGGTG | 189 |
| ≤1X42 | CGAGGGCA | 47 | TGCCCTCG | 88 | ≈2X38 | CACCTTAT | 130 | ATAAGGTG | 190 |
| ≈1X43 | CGGGAATA | 48 | TATTCCCG | 89 | ≈2X39 | CACTCCAT | 131 | ATGGAGTG | 191 |
| ≈1X44 | CGGGAGCT | 49 | AGCTCCCG | 90 | ≈2X40 | CATCCCTA | 132 | TAGGGATG | 192 |
| ≈1X45 | CTGAAGCC | 50 | GGCTTCAG | 91 | ≈2X41 | CCCTCCGG | 133 | CCGGAGGG | 193 |
| ≈1X46 | CTGGAAAC | 51 | GTTTCCAG | 92 | ≈2X42 | CCCTTCTA | 134 | TAGAAGGG | 194 |
| ≈1X47 | GAGAGGGT | 52 | ACCCTCTC | 93 | ≈2X43 | CCCTTTCG | 135 | CGAAAGGG | 195 |
| ≈1X48 | GAGGAACA | 53 | TGTTCCTC | 94 | ≈2X44 | CCTCTCAT | 136 | ATGAGAGG | 196 |
| ≈1X49 | GAGGGAAT | 54 | ATTCCCTC | 95 | ≈2X45 | CCTTTCCC | 137 | GGGAAAGG | 197 |
| ≈1X50 | GCAAAGGG | 55 | CCCTTTGC | 96 | ≈2X46 | CGCTCCCA | 138 | TGGGAGCG | 198 |
| ≈1X51 | GCAGAGAA | 56 | TTCTCTGC | 97 | ≈2X47 | CGTCCCAC | 139 | GTGGGACG | 199 |
| ≈1X52 | GCAGGACC | 57 | GGTCCTGC | 98 | ≈2X48 | CGTCCTGG | 140 | CCAGGACG | 200 |
| ≈1X53 | GCGGAAGT | 58 | ACTTCCGC | 99 | ≈2X49 | CGTCTCCG | 141 | CGGAGACG | 201 |
| ≈1X54 | GCGGGATA | 59 | TATCCCGC | 100 | ≈2X50 | CTCCCTCG | 142 | CGAGGGAG | 202 |
| ≈1X55 | GGAAGAGA | 60 | TCTCTTCC | 101 | ≈2X51 | CTTCCCGT | 143 | ACGGGAAG | 203 |
| ≈1X56 | GGAGAGGT | 61 | ACCTCTCC | 102 | ≈2X52 | GACTCCGC | 144 | GCGGAGTC | 204 |
| ≈1X57 | GGAGGATT | 62 | AATCCTCC | 103 | ≈2X53 | GCCCTCGG | 145 | CCGAGGGC | 205 |
| | | | | 104 | ≈2X54 | GCCCTTCC | 146 | GGAAGGGC | 206 |
| | | | | | ≈2X55 | GCCTCCTT | 147 | AAGGAGGC | 207 |
| | | | | | ≈2X56 | GCTCCCTG | 148 | CAGGGAGC | 208 |
| | | | | | ≈2X57 | GGCCCTAA | 149 | TTAGGGCC | 209 |
| | | | | | ≈2X58 | GGCTCTCG | 150 | CGAGAGCC | 210 |
| | | | | | ≈2X59 | GGCTTCCC | 151 | GGGAAGCC | 211 |
| | | | | | ≈2X60 | GGTCCCGA | 152 | TCGGGACC | 212 |
| | | | | | ≈2X61 | GGTTTCGG | 153 | CCGAAACC | 213 |
| | | | | | ≈2X62 | GTTTCCCG | 154 | CGGGAAAC | 214 |
| | | | | | ≈2X63 | TACCTCTT | 155 | AAGAGGTA | 215 |
| | | | | | ≈2X64 | TACTTCGA | 156 | TCGAAGTA | 216 |
| | | | | | ≈2X65 | TCCCTCAC | 157 | GTGAGGGA | 217 |
| | | | | | ≈2X66 | TCCTTTGT | 158 | ACAAAGGA | 218 |
| | | | | | ≈2X67 | TCTCCTCC | 159 | GGAGGAGA | 219 |
| | | | | | ≈2X68 | TCTTCCTC | 160 | GAGGAAGA | 220 |
| | | | | | ≈2X69 | TGTCCCTT | 161 | AAGGGACA | 221 |
| | | | | | ≈2X70 | TGTCTTCT | 162 | AGAAGACA | 222 |
| | | | | | ≈2X71 | TGTTCTAA | 163 | TTAGAACA | 223 |
| | | | | | ≈2X72 | TTCCCTAT | 164 | ATAGGGAA | 224 |

TABLE 3
Summary of the quality control (QC) for the synthesis of the DEL

| | | Mass Observed (Expected |
|---|---|---|
| Compound ID | Compound Structure | [M + H] with Linker) |
| 1 | 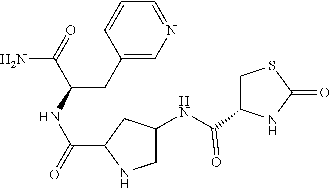 [SMILES: NC(=O)[C@@H](Cc1cccnc1)NC(=O)C1CC(NC(=O)[C@@H]2CSC(=O)N2)CN1] | 1396.8 (1396.6) |
| 2 | 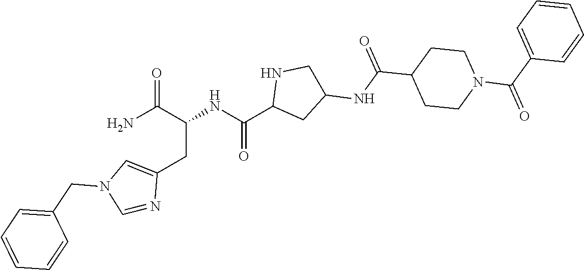 [SMILES: NC(=O)[C@@H](Cc1cn(Cc2ccccc2)cn1)NC(=O)C1CC(NC(=O)C2CCN(C(=O)c3ccccc3)CC2)CN1] | 1562.1 (1561.7) |
| 3 | 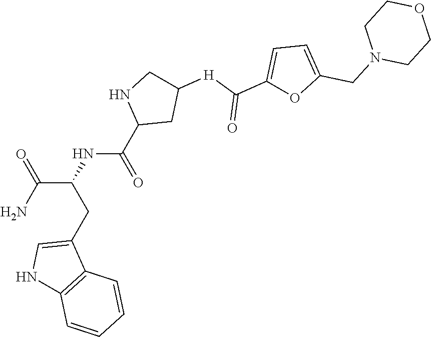 | 1498.9 (1498.7) |
| 4 | 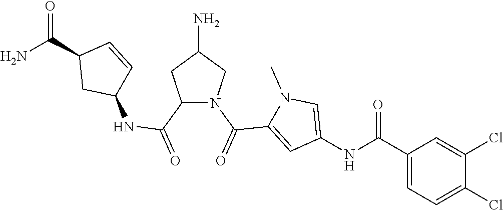 [SMILES: Cn1cc(NC(=O)c2ccc(Cl)c(Cl)c2)cc1C(=O)N1CC(N)CC1C(=O)N[C@@H]1C=C[C@H](C(N)=O)C1] | 1522.9 (1522.6) |
| 5 | 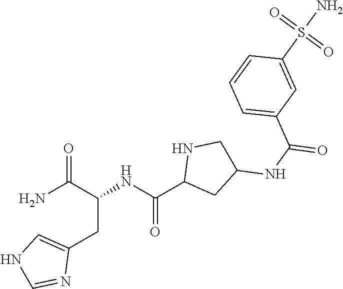 [SMILES: NC(=O)[C@@H](Cc1c[nH]cn1)NC(=O)C1CC(NC(=O)c2cccc(S(N)(=O)=O)c2)CN1] | 1440.0 (1439.6) |
| 6 | 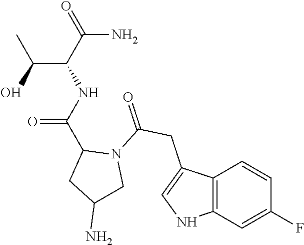 [SMILES: C[C@H](O)[C@@H](NC(=O)C1CC(N)CN1C(=O)Cc1c[nH]c2cc(F)ccc12)C(N)=O] | 1396.0 (1395.6) |
| 7 | 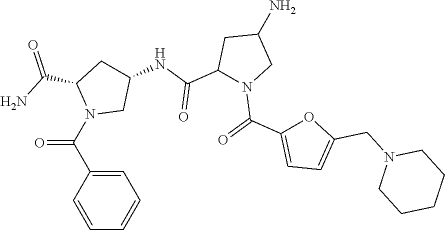 [SMILES: NC(=O)[C@@H]1C[C@H](NC(=O)C2CC(N)CN2C(=O)c2ccc(CN3CCCCC3)o2)CN1C(=O)c1ccccc1] | 1527.0 (1526.7) |
| 8 | 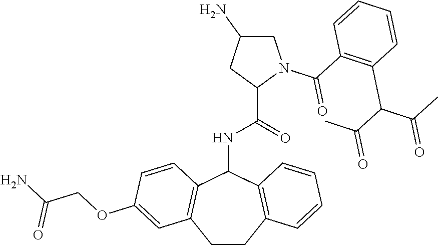 [SMILES: CC(=O)C(C(C)=O)c1ccccc1C(=O)N1CC(N)CC1C(=O)NC1c2ccccc2CCc2cc(OCC(N)=O)ccc21] | 1384.9 (1384.6*) |
| 9 | 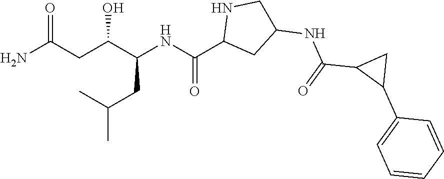 [SMILES: CC(C)C[C@H](NC(=O)C1CC(NC(=O)C2CC2c2ccccc2)CN1)[C@@H](O)CC(N)=O] | 1421.0 (1420.7) |
| 10 | 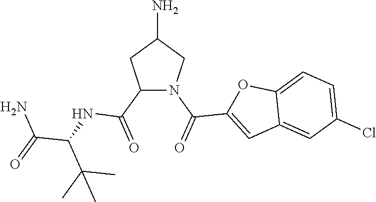 [SMILES: CC(C)(C)[C@@H](NC(=O)C1CC(N)CN1C(=O)c1cc2cc(Cl)ccc2o1)C(N)=O] | 1410.8 (1410.6) |
| 11 | 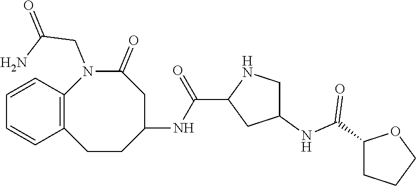 [SMILES: NC(=O)CN1C(=O)CC(NC(=O)C2CC(NC(=O)[C@H]3CCCO3)CN2)CCc2ccccc21] | 1433.8 (1433.6) |
| 12 | 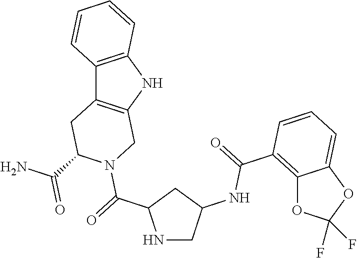 [SMILES: NC(=O)[C@@H]1Cc2c([nH]c3ccccc23)CN1C(=O)C1CC(NC(=O)c2cccc3c2OC(F)(F)O3)CN1] | 1501.9 (1501.6) |
| 13 | 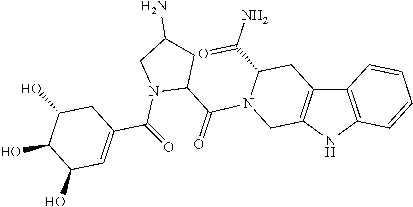 [SMILES: NC(=O)[C@@H]1Cc2c([nH]c3ccccc23)CN1C(=O)C1CC(N)CN1C(=O)C1=C[C@@H](O)[C@@H](O)[C@H](O)C1] | 1473.9 (1473.6) |
| 14 | 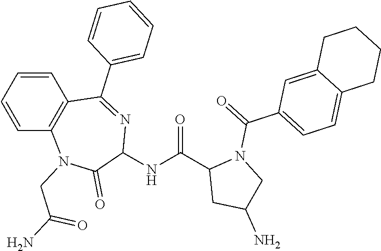 [SMILES: NC(=O)CN1C(=O)C(NC(=O)C2CC(N)CN2C(=O)c2ccc3c(c2)CCCC3)N=C(c2ccccc2)c2ccccc21] | 1568.9 (1568.7) |
| 15 | 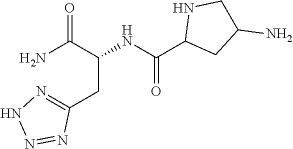 [SMILES: NC(=O)[C@@H](Cc1nn[nH]n1)NC(=O)C1CC(N)CN1] | 1258.8 (1258.6) |
| 16 | 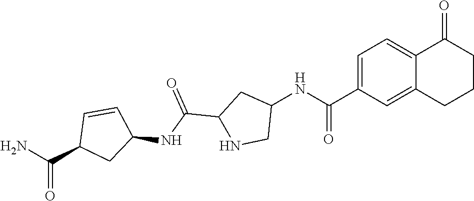 [SMILES: NC(=O)[C@H]1C=C[C@@H](NC(=O)C2CC(NC(=O)c3ccc4c(c3)CCCC4=O)CN2)C1] | 1400.8 (1400.6) |
| 17 | 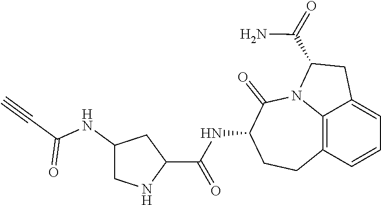 [SMILES: C#CC(=O)NC1CNC(C(=O)N[C@H]2CCc3cccc4c3N(C2=O)[C@H](C(N)=O)C4)C1] | 1417.8 (1449.6) |
| 18 | 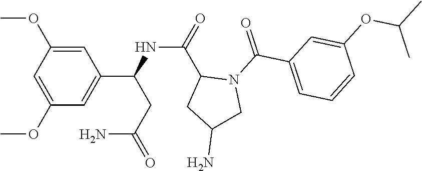 [SMILES: COc1cc(OC)cc([C@H](CC(N)=O)NC(=O)C2CC(N)CN2C(=O)c2cccc(OC(C)C)c2)c1] | 1518.9 (1488.7) |
| 19 | 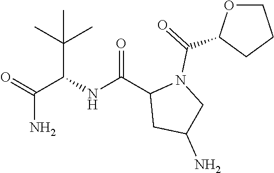 [SMILES: CC(C)(C)[C@H](NC(=O)C1CC(N)CN1C(=O)[C@H]1CCCO1)C(N)=O] | 1330.8 (1330.6) |
| 20 | 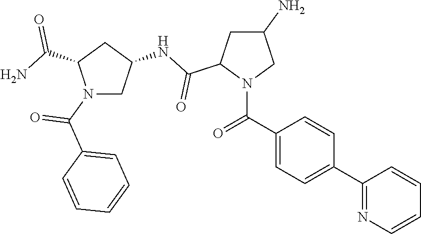 [SMILES: NC(=O)[C@@H]1C[C@H](NC(=O)C2CC(N)CN2C(=O)c2ccc(-c3ccccn3)cc2)CN1C(=O)c1ccccc1] | 1516.7 (1516.7) |

17, 18 = Unmatched

TABLE 4
Physicochemical properties of small molecule libraries.

| Library | MW (Da) | LogP | TPSA (Å) | H Acceptors | H Donors |
|---|---|---|---|---|---|
| DrugBank | 345 ± 198 | 2.0 ± 3.5 | 92 ± 114 | 5.0 ± 6 | 2.0 ± 4 |
| SPDEL - Starting | 493 ± 80 | −0.54 ± 2 | 163 ± 55 | 10.0 ± 2 | 4.0 ± 1 |
| Library | | | | | |
| SPDEL - Hits | 566 ± 51 | −0.87 ± 2 | 168 ± 20 | 6.4 ± 1 | 3.8 ± 1 |
| Inforna compounds | 457 ± 203 | 0.16 ± 5 | 156 ± 118 | 8.6 ± 6 | 5.2 ± 5 |

TABLE S5
Sequences of primers used RT-qPCR.

| | Forward Primer | Reverse Primer |
|---|---|---|
| Gene | (5′→3′) | (5′→3′) |
| RNU6 | ACACGCAAATTCGT | Universal: |
| | GAAGCGTTC | GAATCGAGCACCAGTTACGC |
| | SEQ ID NO: 225 | SEQ ID NO: 233 |
| miR-27a | TTCACAGTGGCT | Universal: |
| | AAGTTCCGC | GAATCGAGCACCAGTTACGC |
| | SEQ ID NO: 226 | SEQ ID NO: 233 |
| miR-23a | ATCACATTGCCA | Universal: |
| | GGGATTTCC | GAATCGAGCACCAGTTACGC |
| | SEQ ID NO: 227 | SEQ ID NO: 233 |
| miR-24 | TGGCTCAGTTC | Universal: |
| | AGCAGGAACAG | GAATCGAGCACCAGTTACGC |
| | SEQ ID NO: 228 | SEQ ID NO: 233 |
| pri-miR- | GAGCAGGGCTT | GTGAACACGACTTGGTGTGG |
| 27a | AGCTGCTT | |
| | SEQ ID NO: 229 | SEQ ID NO: 234 |
| 18S | GTAACCCGTTG | CCATCCAATCGGTAGTAGCG |
| | AACCCCATT | SEQ ID NO: 235 |
| | SEQ ID NO: 230 | |
| FBXW7 | ACTGGAAAGTG | TACTGGGGCTAGGCAAACAA |
| | ACTCTGGGA | SEQ ID NO: 236 |
| | SEQ ID NO: 231 | |
| GAPDH | AAGGTGAAGGT | AATGAAGGGGTCATTGATGG |
| | CGGAGTCAA | SEQ ID NO: 237 |
| | SEQ ID NO: 232 | |

TABLE S6
Sequences of primers and template
for transcription (5′→3′)

| | |
|---|---|
| Template for | CTGAGGAGCAGGGCTTAGCTGC |
| WT pri-miR-27a | TTGTGAGCAGGGTCCACACCAA |
| | GTCGTGTTCACAGTGGCTAAGT |
| | TCCGCCCCCCAG |
| | SEQ ID NO: 238 |
| Template for | CTGAGGAGCAGGGCTTAGCTGC |
| mutated | TTGTGAGCAGGGTCCACACCAA |
| pri-miR-27a | GTCGTGTTCACAGTGGCTAAGT |
| | TCCGCTCCTCAG |
| | SEQ ID NO: 239 |
| Forward primer | TAATACGACTCACTATAGAGAG |
| | AGGCCCCGAAGCCTGTGCCTGG |
| | CCTGAGGAGCAGGGCT |
| | SEQ ID NO: 240 |
| Reverse primer | GGCAAGGCCAGAGGAGGTGAGG |
| for WT | GCCTGGGGGGCGGAACT |
| | SEQ ID NO: 241 |
| Reverse primer | GGCAAGGCCAGAGGAGGTGAGG |
| for mutated | GCCTGAGGAGCGGAACT |
| | SEQ ID NO: 242 |

* Red indicates mutated bases

### REFERENCES

1. K. H. Bleicher, H.-J. Böhm, K. Müller, A. I. Alanine, Hit and lead generation: beyond high-throughput screening. Nat. Rev. Drug Discovery 2, 369-378 (2003).
2. C. A. Lipinski, F. Lombardo, B. W. Dominy, P. J. Feeney, Experimental and computational approaches to estimate solubility and permeability in drug discovery and development settings. Adv. Drug Delivery Rev. 23, 3-25 (1997).
3. D. F. Veber et al., Molecular properties that influence the oral bioavailability of drug candidates. J. Med. Chem. 45, 2615-2623 (2002).
4. C. A. Lipinski, F. Lombardo, B. W. Dominy, P. J. Feeney, Experimental and computational approaches to estimate solubility and permeability in drug discovery and development settings. Adv. Drug Deliv. Rev. 46, 3-26 (2001).
5. D. F. Veber et al., Molecular properties that influence the oral bioavailability of drug candidates. J. Med. Chem. 45, 2615-2623 (2002).
6. M. A. Clark et al., Design, synthesis and selection of DNA-encoded small-molecule libraries. Nat. Chem. Biol. 5, 647-654 (2009).
7. S. Brenner, R. A. Lerner, Encoded combinatorial chemistry. Proc. Natl. Acad. Sci. U.S.A. 89, 5381 (1992).
8. M. W. Kanan, M. M. Rozenman, K. Sakurai, T. M. Snyder, D. R. Liu, Reaction discovery enabled by DNA-templated synthesis and in vitro selection. Nature 431, 545-549 (2004).
9. S. Melkko, J. Scheuermann, C. E. Dumelin, D. Neri, Encoded self-assembling chemical libraries. Nat. Biotechnol. 22, 568-574 (2004).
10. Z. J. Gartner, M. W. Kanan, D. R. Liu, Multistep small-molecule synthesis programmed by DNA templates. J. Am. Chem. Soc. 124, 10304-10306 (2002).
11. R. A. Goodnow, C. E. Dumelin, A. D. Keefe, DNA-encoded chemistry: enabling the deeper sampling of chemical space. Nat. Rev. Drug Discovery 16, 131-147 (2017).
12. D. L. Usanov, A. I. Chan, J. P. Maianti, D. R. Liu, Second-generation DNA-templated macrocycle libraries for the discovery of bioactive small molecules. Nat. Chem. 10, 704-714 (2018).
13. D. Neri, R. A. Lerner, DNA-encoded chemical libraries: a selection system based on endowing organic compounds with amplifiable information. Annu. Rev. Biochem. 87, 479-502 (2018).
14. N. Krall, J. Scheuermann, D. Neri, Small targeted cytotoxics: current state and promises from DNA-encoded chemical libraries. Angew. Chem., Int. Ed. Engl. 52, 1384-1402 (2013).
15. A. B. MacConnell, P. J. McEnaney, V. J. Cavett, B. M. Paegel, DNA-encoded solid-phase synthesis: encoding language design and complex oligomer library synthesis. ACS Comb. Sci. 17, 518-534 (2015).
16. R. E. Kleiner, C. E. Dumelin, D. R. Liu, Small-molecule discovery from DNA-encoded chemical libraries. Chem. Soc. Rev. 40, 5707-5717 (2011).
17. M. D. Disney, B. G. Dwyer, J. L. Childs-Disney, Drugging the RNA world. Cold Spring Harbor Perspect. Biol. 10 (2018).
18. M. D. Disney, Targeting RNA with small molecules to cCapture opportunities at the intersection of chemistry, biology, and medicine. J. Am. Chem. Soc. 141, 6776-6790 (2019).
19. M. D. Disney, B. M. Suresh, R. I. Benhamou, J. L. Childs-Disney, Progress toward the development of the small molecule equivalent of small interfering RNA. Curr. Opin. Chem. Biol. 56, 63-71 (2020).
20 C. S. Eubanks, J. E. Forte, G. J. Kapral, A. E. Hargrove, Small molecule-based pattern recognition to classify RNA structure. J. Am. Chem. Soc. 139, 409-416 (2017).
21. C. M. Connelly, M. H. Moon, J. S. Schneekloth, Jr., The emerging Role of RNA as a therapeutic target for small molecules. Cell Chem. Biol. 23, 1077-1090 (2016).
22. M. D. Disney et al., Two-dimensional combinatorial screening identifies specific aminoglycoside-RNA internal loop partners. J. Am. Chem. Soc. 130, 11185-11194 (2008).
23. S. Chirayil, R. Chirayil, K. J. Luebke, Discovering ligands for a microRNA precursor with peptoid microarrays. Nucleic Acids Res. 37, 5486-5497 (2009).
24. K. R. Mendes et al., High-throughput identification of DNA-encoded IgG ligands that distinguish active and latent Mycobacterium tuberculosis infections. ACS Chem. Biol. 12, 234-243 (2017).
25. W. G. Cochrane et al., Activity-based DNA-encoded library sScreening. ACS Comb. Sci. 21, 425-435 (2019).
26 A. L. Hackler, F. G. FitzGerald, V. Q. Dang, A. L. Satz, B. M. Paegel, Off-DNA DNA-encoded library affinity screening. ACS Comb. Sci. 22, 25-34 (2020).
27. J. M. Bevilacqua, P. C. Bevilacqua, Thermodynamic analysis of an RNA combinatorial library contained in a short hairpin. Biochemistry 37, 15877-15884 (1998).
28 T. Sander, J. Freyss, M. von Korff, C. Rufener, DataWarrior: an open-source program for chemistry aware data visualization and analysis. J. Chem. Inf. Model. 55, 460-473 (2015).
29. A. B. MacConnell, B. M. Paegel, Poisson statistics of combinatorial library sampling predict false discovery rates of screening. ACS Comb.l Sci. 19, 524-532 (2017).
30. H. C. Kolb, M. G. Finn, K. B. Sharpless, Click chemistry: diverse chemical function from a few good reactions. Angew. Chem., Int. Ed. Engl. 40, 2004-2021 (2001).
31. S. P. Velagapudi et al., Defining RNA-small molecule affinity landscapes enables design of a small molecule inhibitor of an oncogenic noncoding RNA. ACS Cent. Sci. 3, 205-216 (2017).
32 B. Liu et al., Analysis of secondary structural elements in human microRNA hairpin precursors. BMC Bioinformatics 17, 112 (2016).
33. T. D. Schneider, R. M. Stephens, Sequence logos: a new way to display consensus sequences. Nucleic Acids Res. 18, 6097-6100 (1990).
34 M. Nettling et al., DiffLogo: a comparative visualization of sequence motifs. BMC Bioinformatics 16, 387 (2015).
35 S. Griffiths-Jones, R. J. Grocock, S. van Dongen, A. Bateman, A. J. Enright, miRBase: microRNA sequences, targets and gene nomenclature. Nucleic Acids Res. 34, D140-D144 (2006).
36. G. Jiang, W. Shi, H. Fang, X. Zhang, miR27a promotes human breast cancer cell migration by inducing EMT in a FBXW7dependent manner. Mol. Med. Rep. 18, 5417-5426 (2018).
37 C. E. Fletcher et al., Androgen-regulated processing of the oncomir MiR-27a, which targets Prohibitin in prostate cancer. Hum. Mol. Genet. 21, 3112-3127 (2012).
38. W. Tang et al., MiR-27 as a prognostic marker for breast cancer progression and patient survival. Plos One 7, e51702 (2012).
39. X. Li et al., MicroRNA-27a indirectly regulates Estrogen Receptor a expression and hormone responsiveness in MCF-7 breast cancer cells. Endocrinology 151, 2462-2473 (2010).
40. Y. Sun, X. Yang, M. Liu, H. Tang, B4GALT3 up-regulation by miR-27a contributes to the oncogenic activity in human cervical cancer cells. Cancer Lett. 375, 284-292 (2016).
41. R. Chhabra, R. Dubey, N. Saini, Cooperative and individualistic functions of the microRNAs in the miR-23a˜27a˜24-2 cluster and its implication in human diseases. Mol. Cancer Res. 9, 232 (2010).
42. A. H. Buck et al., Post-transcriptional regulation of miR-27 in murine cytomegalovirus infection. RNA (New York, N.Y.) 16, 307-315 (2010).
43. J. Y. Lee et al., Development of a dual-luciferase reporter system for in vivo visualization of microRNA biogenesis and posttranscriptional regulation. J. Nucl. Med. 49, 285-294 (2008).
44 S. Zhu et al., MicroRNA-21 targets tumor suppressor genes in invasion and metastasis. Cell Res. 18, 350-359 (2008).
45. S. K. Surapaneni, Z. R. Bhat, K. Tikoo, MicroRNA-941 regulates the proliferation of breast cancer cells by altering histone H₃ Ser 10 phosphorylation. Sci. Rep. 10, 17954 (2020).
46. S. Jiang et al., MicroRNA-155 functions as an oncomiR in beast cancer by targeting the Suppressor of Cytokine Signaling 1 gene. Cancer Res. 70, 3119 (2010).
47. G. K. Scott, M. D. Mattie, C. E. Berger, S. C. Benz, C. C. Benz, Rapid alteration of microRNA levels by histone deacetylase inhibition. Cancer Res. 66, 1277-1281 (2006).
48. S. U. Mertens-Talcott, S. Chintharlapalli, X. Li, S. Safe, The oncogenic microRNA-27a targets genes that regulate specificity protein transcription factors and the G2-M checkpoint in MDA-MB-231 breast cancer cells. Cancer Res. 67, 11001-11011 (2007).
49. V. Agarwal, G. W. Bell, J. W. Nam, D. P. Bartel, Predicting effective microRNA target sites in mammalian mRNAs. eLife 4, e05005 (2015).
50. A. N. Santhanam, A. R. Baker, G. Hegamyer, D. A. Kirschmann, N. H. Colburn, Pdcd4 repression of lysyl oxidase inhibits hypoxia-induced breast cancer cell invasion. Oncogene 29, 3921-3932 (2010).
51. H. N. Mohammed, M. R. Pickard, M. Mourtada-Maarabouni, The protein phosphatase 4-PEA15 axis regulates the survival of breast cancer cells. Cell. Signalling 28, 1389-1400 (2016).
52. M. G. Costales et al., Small-molecule targeted recruitment of a nuclease to cleave an oncogenic RNA in a mouse model of metastatic cancer. Proc. Natl. Acad. Sci. U.S.A 117, 2406-2411 (2020).
53. D. Szklarczyk et al., STRING v11: protein-protein association networks with increased coverage, supporting functional discovery in genome-wide experimental datasets. Nucleic Acids Res. 47, D607-D613 (2019).
54. G. A. Brar, J. S. Weissman, Ribosome profiling reveals the what, when, where and how of protein synthesis. Nat. Rev. Mol. Cell Biol. 16, 651-664 (2015).
55. N. T. Ingolia, S. Ghaemmaghami, J. R. S. Newman, J. S. Weissman, Genome-wide analysis in vivo of translation with nucleotide resolution using ribosome profiling. Science 324, 218 (2009).
56 A. Regev et al., The human cell atlas. eLife 6, e27041 (2017).
57. Evan Z. Macosko et al., Highly parallel genome-wide expression profiling of individual cells using nanoliter droplets. Cell 161, 1202-1214 (2015).
58. C. Tuerk, L. Gold, Systematic evolution of ligands by exponential enrichment: RNA ligands to bacteriophage T4 DNA polymerase. Science 249, 505-510 (1990).
59 A. A. Beaudry, G. F. Joyce, Directed evolution of an RNA enzyme. Science 257, 635-641 (1992).
60. G. F. Joyce, RNA evolution and the origins of life. Nature 338, 217-224 (1989).
61. A. D. Ellington, J. W. Szostak, In vitro selection of RNA molecules that bind specific ligands. Nature 346, 818-822 (1990).
62. A. Litovchick et al., Novel nucleic acid binding small molecules discovered using DNA-encoded Cchemistry. Molecules 24, 2026 (2019).

### EXPERIMENTAL SECTION REFERENCES

1. M. D. Disney et al., Inforna 2.0: A platform for the sequence-based design of small molecules targeting structured RNAs. ACS Chem. Biol. 11, 1720-1728 (2016).
2. T. D. Schneider, R. M. Stephens, Sequence logos: a new way to display consensus sequences. Nucleic Acids Res. 18, 6097-6100 (1990).
3. M. Nettling et al., DiffLogo: a comparative visualization of sequence motifs. BMC Bioinformatics 16, 387 (2015).
4. V. Agarwal, G. W. Bell, J. W. Nam, D. P. Bartel, Predicting effective microRNA target sites in mammalian mRNAs. eLife 4 (2015).
5. A. B. MacConnell, P. J. McEnaney, V. J. Cavett, B. M. Paegel, DNA-encoded solid-phase synthesis: encoding language design and complex oligomer library synthesis. ACS Comb. Sci. 17, 518-534 (2015).
6. W. G. Cochrane et al., Activity-based DNA-encoded library screening. ACS Comb. Sci. 21, 425-435 (2019).
7. K. R. Mendes et al., High-throughput identification of DNA-encoded IgG ligands that distinguish active and latent Mycobacterium tuberculosis infections. ACS Chem. Biol. 12, 234-243 (2017).
8. T. Sander, J. Freyss, M. von Korff, C. Rufener, DataWarrior: An open-source program for chemistry aware data visualization and analysis. J. Chem. Inf. Model. 55, 460-473 (2015).
9. T. Tran, M. D. Disney, Two-dimensional combinatorial screening of a bacterial rRNA A-site-like motif library: defining privileged asymmetric internal loops that bind aminoglycosides. Biochemistry 49, 1833-1842 (2010).
10. S. P. Velagapudi et al., Defining RNA-small molecule affinity landscapes enables design of a small molecule inhibitor of an oncogenic noncoding RNA. ACS Cent. Sci. 3, 205-216 (2017).
11. M. G. Costales, Y. Matsumoto, S. P. Velagapudi, M. D. Disney, Small molecule targeted recruitment of a nuclease to RNA. J. Am. Chem. Soc. 140, 6741-6744 (2018).
12. K. J. Livak. T. D. Schmittgen, Analysis of relative gene expression data using real-time quantitative PCR and the 2 (-Delta Delta C (T)) Method. Methods (San Diego, Calif.) 25, 402-408 (2001).

### SUMMARY STATEMENTS

The inventions, examples, biological assays and results described and claimed herein have may attributes and embodiments include, but not limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, textbook or other referenced material or document. The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

What is claimed is:

1. A DEL method for screening compounds by DNA encoded labeling, comprising:
Forming an immobilized library of compounds having DNA labels wherein each of the compounds is bound to an individual solid microsupport and each microsupport also carries a unique DNA sequence that describes both the microsupport and the synthesis history of the individual compound immobilized on the microsupport;
Contacting the immobilized compound library with a first RNA library comprising synthesized RNA constructs with fluorescent labels wherein the synthesized RNA constructs display a randomized region in a discrete structural motif pattern, and simultaneously contacting the immobilized compound library with a non-selective RNA counter construct labeled with tetramethylrhodamine wherein the non-selective RNA counter construct comprises the synthesized RNA library construct in which the randomized region is replaced with base paired nucleotides, to produce a first subgroup of the immobilized compound library bound to the first RNA library, a second subgroup of the immobilized compound library bound to the second non-selective RNA counter construct, and a third unbound subgroup;
Sorting and isolating the first, second and third subgroups by flow cytometry using fluorescent detection;
Amplifying the DNA labels of each of compounds of the first subgroup;
Reading the amplified DNA labels to identify each of the compounds of the first subgroup.

2. A method according to claim 1 wherein the first RNA library of synthesized RNA constructs with fluorescent labels comprises a group of about 4,096 DY647 3×3 ILL synthesized RNA members with the randomized 3×3 nucleotide internal loop pattern.

3. A method according to claim 1 or 2 further comprising sequencing the RNA constructs associated with the compounds of the first subgroup to determine a first occurrence frequency of synthesized RNA constructs relative to each compound.

4. A method according to any of claims 1-3 further comprising a 2DCS technique comprising:
Conjugating each of the compounds of the first subgroup to individual immobilized sites in a microarray to produce a microarray of conjugated compounds;
Contacting the microarray of conjugated compounds with a second radioactively labeled RNA library and with competitor oligonucleotides to produce a microarray of at least some conjugated compounds bound to certain members of the second RNA library and at least some conjugated compounds bound to certain competitor oligonucleotides, wherein the second RNA library comprises the synthesized RNA library display a randomized region in a discrete structural motif pattern of the first library without the fluorescent labels and with radioactive phosphorus labels, and the competitor oligonucleotides comprise nucleotide sequences that mimic regions common to all members of both RNA libraries;
Washing the microarray to remove unbound synthesized RNA constructs from the library and unbound competitor oligonucleotides,
Harvesting the bound synthesized RNA constructs of the microarray displaying radioactivity, and reading the synthesized RNA construct sequences to produce a sequence data set;
Analyzing the sequence data set to determine the frequency of each harvested synthesized RNA construct;
Comparing the frequency of an RNA selected from the microarray with its frequency in the synthesized RNA library that did not undergo the 2DCS selection process to provide a statistical occurrence hierarchy of bound RNA;
Determining an RNA from the statistical occurrence hierarchy that will selectively bind with a compound by application of the statistical hierarchy to cellular RNAs.

5. The method of claim 4, further comprising obtaining RNA that binds to each compound to generate a series of RNA samples, amplifying each RNA sample in the series, sequencing each RNA sample in the series, or any combination thereof.

6. The method of claim 4, wherein the dataset of identified bound RNA motif-compound pairs comprises a structural description of each RNA motif, a listing of which RNA motif binds to each compound, one or more RNA sequence for each RNA motif, a description of each RNA motif's 2-dimensional and/or three-dimensional structure, a description of each RNA motif as single-stranded or double-stranded, a description of each RNA motif as an internal loop, hairpin loop, a bulge, a bubble, or a branch, or any combination thereof.

7. The method of claim 4, wherein the dataset of identified bound RNA motif-compound pairs comprises a description of each RNA motif as an RNA symmetric internal loop, asymmetric internal loop, 1×1 internal loop, 1×2 internal loop, 1×3 internal loop, 2×2 internal loop, 2×3 internal loop, 2×4 internal loop, 3×3 internal loop, 3×4 internal loop, 4×4 internal loop, 4×5 internal loop, 5×5 internal loop, 1 base bulge, 2 base bulge, 3 base bulge, 4 base bulge, 5 base bulge, 4 base hairpin loop, 5 base hairpin loop, 6 base hairpin loop, 7 base hairpin loop, 8 base hairpin loop, 9 base hairpin loop, 10 base hairpin loop, multi-branch loop, or pseudoknot.

8. The method of claim 4, wherein the dataset of identified bound RNA motif-compound pairs comprises a structural description of each small molecule, a description of each compound by chemical formula, chemical name, a description of each compound structure, a description of each compound three-dimensional structure, a description of each compound three-dimensional atomic structure, or a combination thereof.

9. The method of claim 4, wherein the dataset of identified bound RNA motif-compound pairs comprises a description of bonds formed between RNA motifs and compounds, a description of alignments for each structural feature of each RNA motif with each compound to which the RNA motif binds, a description of alignments for each structural feature of compound with each structural feature of the RNA motif to which the compound binds, of any combination thereof.

10. The method of claim 4, wherein comparing the query dataset of RNA secondary structures from the RNA, with the dataset of identified bound RNA motif-compound pairs, comprises:
(a) aligning one or more structural feature of each RNA secondary structure with one or more structural feature of one or more of the RNA motifs;
(b) a series of alignments for each structural feature of each RNA secondary structure with one or more structural feature of one or more of the RNA motifs;
(c) a series of alignments for each structural feature of each RNA secondary structure with one or more structural feature of one or more of the RNA motifs until a best-fit RNA motif is identified that optimally corresponds with RNA secondary structure;
(d) a series of alignments for each structural feature of each RNA secondary structure with one or more structural feature of one or more of the RNA motifs until a best-fit compound-RNA motif pair is identified, where the RNA motif of the pair has a structure that optimally corresponds with RNA secondary structure; or
(e) any combination thereof.

11. The method of claim 4, wherein the method identifies a compound that binds to the RNA by providing an output listing at least one RNA secondary structure from the RNA, and a compound that binds to the at least one RNA secondary structure.

12. The method of claim 4, wherein the RNA is any cellular RNA, for example, a microRNA, a tRNA, a rRNA, a lncRNA, or a small interfering RNA.

13. A method according to any of claims 1-12 wherein the compounds are peptide compounds of multiple natural and/or synthetic amino acid units, preferably at least 3 units, more preferably up to about 8 units, especially more preferably up to about 5 or 6 units, produced by Merrifield synthesis on the microsupports wherein each microsupport carries a single peptide compound and the unique DNA codon label with PCR primer amplification sequence and the amino acid units of the peptide compounds are selected from a library of at least 200 natural a amino acid units and synthetic a, B, y, 8 amino acid units having alkyl, aryl, arylalkyl, heteroaryl, heteroarylalkyl cycloalkyl, heterocycloalkyl side chains optionally substituted by amine, amide, carboxyl, halo groups and having nitrogen, oxygen and/or sulfur as bivalent atoms in the carbon-carbon links of the alkyl, aryl, heteroaryl, cycloalkyl and the like groups.

14. A method according to any of claims 1-13 wherein the DNA labels with PCR primer binding sites are bound to microsupports by enzymatic coupling.

15. A method according to any of claim 13 or 14 wherein the peptide compounds are formed from natural and synthetic amino acid unit monomers tagged with Merrifield peptide coupling groups wherein groups of the natural and synthetic amino acid unit monomers that are not to be coupled are protected.

16. A method according to any of claims 13-15 wherein the peptide compounds are bound to the microsupports by an amide formation with the carboxyl group of the hub amino acid unit monomer.

17. A method according to any of claims 13-16 wherein the peptide compounds are conjugated to microarray wells by a triazole group formed from combination of a propargyl group bound to the peptide compounds and an azide group bound to the microarray wells.

18. A method for targeting primary microRNA-27a (pri-miR-27a) or microRNA-409 (miR-409) comprising contacting pri-miR-27a or miR-409 with a peptide compound of Formula I 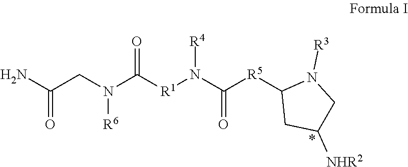
wherein
R⁶ is hydrogen, C1-C3 alkyl or propargyl;
R¹ is CHR⁷ wherein R⁷ is phenyl, butyl, isobutyl, 2-cyanophenyl, CH₂CH (CH₂ (3-indolyl)) or R⁷ and R⁴ together form an azetidine ring;
R⁴ is hydrogen or together with R⁷ forms the azetidine ring;
R⁵ is a bond between carbonyl and the pyrrolidine ring or is CHR⁸NHCO— wherein R⁸ and R⁴ together form a pyrrolidinone ring provided that R⁴ cannot be both of the azetidine ring and the pyrrolidinone ring;
R³ is hydrogen or —CO—R⁹ wherein R⁹ is 5-trifluoromethylbenzothiophen-2-yl, 5-chloroindol-3-yl or 2-(4-morpholinylcarbonyl)phenyl provided that R³ is hydrogen when R¹ is CHR⁷ and R⁷ is 2-cyanophenyl or CH₂CH (CH₂(3-indolyl));
R² is hydrogen or COR¹⁰ wherein R¹⁰ is tetrahydrofuran-2-yl, or quinazolindion-3-ylmethylenyl, provided that R² is not hydrogen when R³ is hydrogen; and, the asterisk indicates the position of the diastereomeric R and S forms of Formula I.

19. A method according to claim 18 wherein the compound of Formula I comprises the peptide compound of Formulas pc1(S), pc2(R) diastereomers, pc3(S), pc4(R) diastereomers, pc5(S), pc6(R) diastereomers, pc7(S), pc8(R) diastereomers and/or pc9(S), pc10(R) diastereomers:

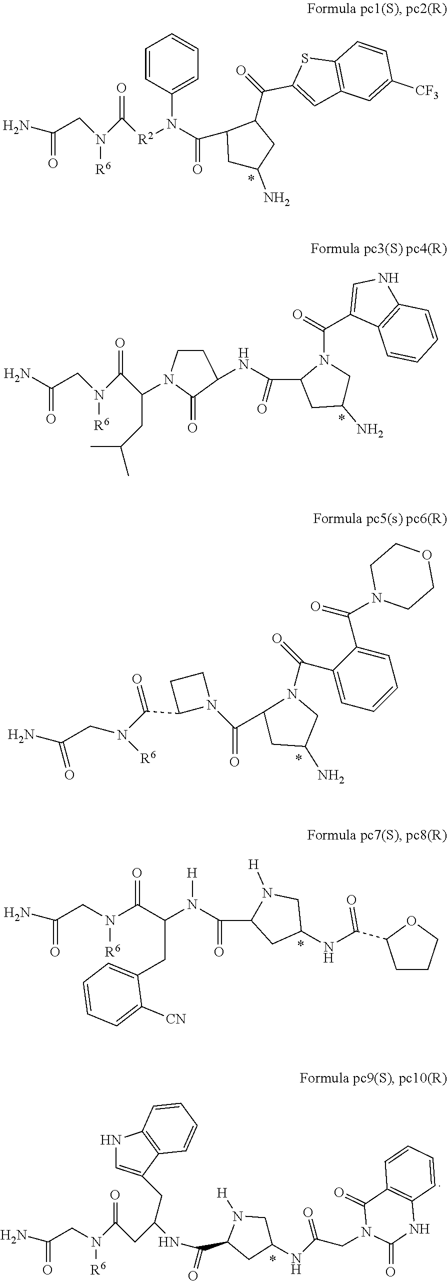

20. A method according to claim 18 or 19 wherein the contacting selectively binds the peptide compound and pri-miR-27a or pri-miR-409.

21. A method according to claim 20 wherein the binding inhibits and/or suppresses Drosha nuclease action upon pri-miR-27a and pri-miR-409.

22. A method according to any of the preceding claims 18-21 wherein the binding is selective for the Drosha processing site 5′GAG/3′CCC but the adjacent 5CAG/3′GCC site does not bind and the peptide compounds are Formulas pc5(S) diastereomer, pc7(S) diastereomer and pc9(S) diastereomer.

23. A method according to claim 22 wherein the microRNA is pri-miR-27a.

24. A method according to any of the preceding claims 18-23 wherein pri-miR-27a is present in MCF-10a cells transfected with a plasmid encoding WT pre-miR-27a and the peptide compound is pd9 S diastereomer.

25. A method according to any of the preceding claims 18-24 wherein the contacting is with MDA-MB-231 cells, MCF-7 cells, LNCaP cells and/or HeLa cells and the peptide compound is pd9 S diastereomer.

26. A method according to claim 25 wherein the MDA-MB-231 cells, MCF-7 cells, LNCaP cells and/or HeLa cells are present in an animal.

27. A method according to claim 26 wherein peptide compound pc9S diastereomer is administered to the animal as a pharmaceutical composition comprising the peptide compound pd5 diastereomer in combination with a pharmaceutically acceptable carrier.

28. A method according to claim 27 wherein peptide compound pc9S diastereomer is administered to the human patients in which disease is caused by overexpression of miR-27a or miR-409 as a pharmaceutical composition comprising the peptide compound pd5 diastereomer in combination with a pharmaceutically acceptable carrier.

29. A composition comprising a peptide compound of Formula I 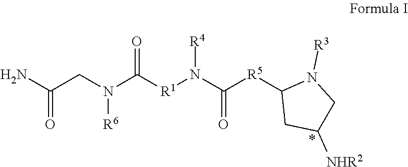
wherein
R⁶ is hydrogen, C1-C3 alkyl or propargyl;
R¹ is CHR⁷ wherein R⁷ is phenyl, butyl, isobutyl, 2-cyanophenyl, CH₂CH (CH₂ (3-indolyl)) or R⁷ and R⁴ together form an azetidine ring;
R⁴ is hydrogen or together with R⁷ forms the azetidine ring;
R⁵ is a bond between carbonyl and the pyrrolidine ring or is CHR⁸NHCO— wherein R⁸ and R⁴ together form a pyrrolidinone ring provided that R⁴ cannot be both of the azetidine ring and the pyrrolidinone ring;
R³ is hydrogen or —CO—R⁹ wherein R⁹ is 5-trifluoromethylbenzothiophen-2-yl, 5-chloroindol-3-yl or 2-(4-morpholinylcarbonyl)phenyl provided that R³ is hydrogen when R¹ is CHR⁷ and R⁷ is 2-cyanophenyl or CH₂CH (CH₂ (3-indolyl));
R² is hydrogen or COR¹⁰ wherein R¹⁰ is tetrahydrofuran-2-yl, or quinazolindion-3-ylmethylenyl, provided that R² is not hydrogen when R³ is hydrogen; and,
the asterisk indicates the position of the diastereomeric R and S forms of Formula I.

30. A composition according to claim 29 wherein the compound of Formula I comprises a peptide compound of Formulas pc1(S), pc2(R) diastereomers, pc3(S), pc4(R) diastereomers, pc5(S), pc6(R) diastereomers, pc7(S), pc8(R) diastereomers and/or pc9(S), pc10(R) diastereomers:

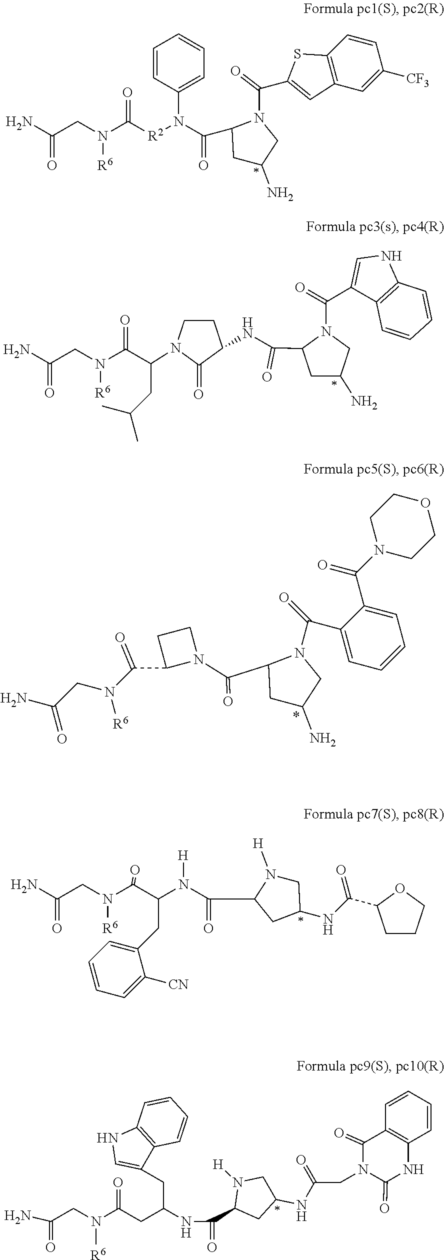

31. A composition according to claim 30 wherein the peptide compound is the S diastereomer of pc5(S), pc7(S) or pc9(S).

32. A composition according to claim 31 wherein the peptide compound is the S diastereomer of pc9(S).


SEQUENCE LISTING (310 sequences)

SEQ ID NO: 1 - DNA, 27 bp, synthetic construct
  source 1..27: mol_type = other DNA
gccgccgccttcgtccttctcagcgac

SEQ ID NO: 2 - DNA, 25 bp, synthetic construct
  source 1..25: mol_type = other DNA
gtggcacaacaactggcgggcaaac

SEQ ID NO: 3 - DNA, 25 bp, synthetic construct
  source 1..25: mol_type = other DNA
gtggcacaacaactggcgggcaaac

SEQ ID NO: 4 - DNA, 17 bp, synthetic construct
  source 1..17: mol_type = other DNA
gttttcccagtcacgac

SEQ ID NO: 5 - DNA, 15 bp, synthetic construct
  source 1..15: mol_type = other DNA
gtggcacaacaactg

SEQ ID NO: 6 - DNA, 100 bp, synthetic construct
  source 1..100: mol_type = other DNA
cgccagggttttcccagtcacgaccaaccacccaaaccacaaacccaaaccccaaacccaacacacaacaacagccgccgccttcgtccttctcagcgac

SEQ ID NO: 7 - DNA, 24 bp, synthetic construct
  source 1..24: mol_type = other DNA
cgccagggttttcccagtcacgac

SEQ ID NO: 8 - DNA, 27 bp, synthetic construct
  source 1..27: mol_type = other DNA
gccgcccagtcctgctcgcttcgctac

SEQ ID NO: 9 - DNA, 89 bp, synthetic construct
  source 1..89: mol_type = other DNA
atggnnnnnnnntcannnnnnnngttnnnnnnnnctannnnnnnnttcnnnnnnnncgcnnnnnnnngcctcccaaacnnnnnnnngtt

SEQ ID NO: 10 - skipped

SEQ ID NO: 11 - DNA, 27 bp, synthetic construct
  source 1..27: mol_type = other DNA
gccgccgccttcgtccttctcagcgac

SEQ ID NO: 12 - DNA, 25 bp, synthetic construct
  source 1..25: mol_type = other DNA
gtggcacaacaactggcgggcaaac

SEQ ID NO: 13 - DNA, 50 bp, synthetic construct
  source 1..50: mol_type = other DNA
cctctctatgggcagtcggtgatgccgccgccttcgtccttctcagcgac

SEQ ID NO: 14 - DNA, 70 bp, synthetic construct
  source 1..70: mol_type = other DNA
ccatctcatccctgcgtgtctccgactcagnnnnnnnnnngatgccgcccagtcctgctcgcttcgctac

SEQ ID NO: 15 - DNA, 70 bp, synthetic construct; identical to SEQ ID NO: 14
  source 1..70: mol_type = other DNA

SEQ ID NO: 16 - RNA, 19 nt, synthetic construct
  source 1..19: mol_type = other RNA
ctgaggtgaaacatcccag

SEQ ID NO: 17 - RNA, 19 nt, synthetic construct
  source 1..19: mol_type = other RNA
ctcaggtgaaacatccgag

SEQ ID NO: 18 - DNA, 27 bp, synthetic construct
  source 1..27: mol_type = other DNA
gccgccgccttcgtccttctcagcgac

SEQ ID NO: 19 - DNA, 33 bp, synthetic construct
  source 1..33: mol_type = other DNA
ccatgtcgctgagaaggacgaaggcggcggcgg

SEQ ID NO: 20 - DNA, 44 bp, synthetic construct
  source 1..44: mol_type = other DNA
gcctcccaaacnnnnnnnngtttgcccgccagttgttgtgccac

SEQ ID NO: 21-224 - skipped (204 entries)

SEQ ID NO: 225 - DNA, 23 bp, synthetic construct
  source 1..23: mol_type = other DNA
acacgcaaattcgtgaagcgttc

SEQ ID NO: 226 - DNA, 21 bp, synthetic construct
  source 1..21: mol_type = other DNA
ttcacagtggctaagttccgc

SEQ ID NO: 227 - DNA, 21 bp, synthetic construct
  source 1..21: mol_type = other DNA
atcacattgccagggatttcc

SEQ ID NO: 228 - DNA, 22 bp, synthetic construct
  source 1..22: mol_type = other DNA
tggctcagttcagcaggaacag

SEQ ID NO: 229 - DNA, 19 bp, synthetic construct
  source 1..19: mol_type = other DNA
gagcagggcttagctgctt

SEQ ID NO: 230 - DNA, 20 bp, synthetic construct
  source 1..20: mol_type = other DNA
gtaacccgttgaaccccatt

SEQ ID NO: 231 - DNA, 20 bp, synthetic construct
  source 1..20: mol_type = other DNA
actggaaagtgactctggga

SEQ ID NO: 232 - DNA, 20 bp, synthetic construct
  source 1..20: mol_type = other DNA
aaggtgaaggtcggagtcaa

SEQ ID NO: 233 - DNA, 20 bp, synthetic construct
  source 1..20: mol_type = other DNA
gaatcgagcaccagttacgc

SEQ ID NO: 234 - DNA, 20 bp, synthetic construct
  source 1..20: mol_type = other DNA
gtgaacacgacttggtgtgg

SEQ ID NO: 235 - DNA, 20 bp, synthetic construct
  source 1..20: mol_type = other DNA
ccatccaatcggtagtagcg

SEQ ID NO: 236 - DNA, 20 bp, synthetic construct
  source 1..20: mol_type = other DNA
tactggggctaggcaaacaa

SEQ ID NO: 237 - DNA, 20 bp, synthetic construct
  source 1..20: mol_type = other DNA
aatgaaggggtcattgatgg

SEQ ID NO: 238 - DNA, 78 bp, synthetic construct
  source 1..78: mol_type = other DNA
ctgaggagcagggcttagctgcttgtgagcagggtccacaccaagtcgtgttcacagtggctaagttccgccccccag

SEQ ID NO: 239 - DNA, 78 bp, synthetic construct
  source 1..78: mol_type = other DNA
ctgaggagcagggcttagctgcttgtgagcagggtccacaccaagtcgtgttcacagtggctaagttccgctcctcag

SEQ ID NO: 240 - DNA, 60 bp, synthetic construct
  source 1..60: mol_type = other DNA
taatacgactcactatagagagaggccccgaagcctgtgcctggcctgaggagcagggct

SEQ ID NO: 241 - DNA, 39 bp, synthetic construct
  source 1..39: mol_type = other DNA
ggcaaggccagaggaggtgagggcctggggggcggaact

SEQ ID NO: 242 - DNA, 39 bp, synthetic construct
  source 1..39: mol_type = other DNA
ggcaaggccagaggaggtgagggcctgaggagcggaact

SEQ ID NO: 243-282 - skipped (40 entries)

SEQ ID NO: 283 - RNA, 46 nt, synthetic construct
  source 1..46: mol_type = other RNA
gggagagggtttaatnnntacgaaagtannnattggatccgcaagg

SEQ ID NO: 284 - RNA, 78 nt, synthetic construct
  source 1..78: mol_type = other RNA
gtgaggagcagggcttagctgcttgtgagcagggtccacaccaagtcgtgttcacagtggctaagttccgccccccag

SEQ ID NO: 285 - RNA, 62 nt, synthetic construct
  source 1..62: mol_type = other RNA
agggcttagctgcttgtgagcagggtccacaccaagtcgtgttcacagtggctaagttccgc

SEQ ID NO: 286 - RNA, 21 nt, synthetic construct
  source 1..21: mol_type = other RNA
ttcacagtggctaagttccgc

SEQ ID NO: 287 - RNA, 22 nt, synthetic construct
  source 1..22: mol_type = other RNA
ctgaggagcagccgccccccag

SEQ ID NO: 288 - RNA, 22 nt, synthetic construct
  source 1..22: mol_type = other RNA
ctgaggagcagccgctcctcag

SEQ ID NO: 289 - RNA, 10 nt, synthetic construct
  source 1..10: mol_type = other RNA
tctttaccta

SEQ ID NO: 290 - RNA, 10 nt, synthetic construct
  source 1..10: mol_type = other RNA
tacctatgga

SEQ ID NO: 291 - RNA, 10 nt, synthetic construct
  source 1..10: mol_type = other RNA
tatataatga

SEQ ID NO: 292 - RNA, 36 nt, synthetic construct
  source 1..36: mol_type = other RNA
gggagagggtttaaacgaaagtttggatccgcaagg

SEQ ID NO: 293 - RNA, 10 nt, synthetic construct
  source 1..10: mol_type = other RNA
tagataccca

SEQ ID NO: 294 - RNA, 10 nt, synthetic construct
  source 1..10: mol_type = other RNA
tttctacata

SEQ ID NO: 295 - RNA, 78 nt, synthetic construct
  source 1..78: mol_type = other RNA
ctgaggagcagggcttagctgcttgtgagcagggtccacaccaagtcgtgttcacagtggctaagttccgccccccag

SEQ ID NO: 296 - RNA, 79 nt, synthetic construct
  source 1..79: mol_type = other RNA
tggtactcggggagaggttacccgagcaactttgcatctggacgacgaatgttgctcggtgaaccccttttcggtatca

SEQ ID NO: 297 - RNA, 99 nt, synthetic construct
  source 1..99: mol_type = other RNA
tccagccagagcccagcagcccctgagaaccacgtctgctctgagctgggtactgcctgttcagaacaaatgccggttcccagacgctgccagctggcc

SEQ ID NO: 298 - RNA, 65 nt, synthetic construct
  source 1..65: mol_type = other RNA
gggaggagggaggagatgggccagttccctctggctggaacgcccttcccccccttcttcacctg

SEQ ID NO: 299 - RNA, 88 nt, synthetic construct
  source 1..88: mol_type = other RNA
tgaggatgggagctgagggctgggtctttgcgggcgagatgagggtgtcggatcaactggcctacaaagtcccagttctcggcccccg

SEQ ID NO: 300 - RNA, 96 nt, synthetic construct
  source 1..96: mol_type = other RNA
tgtatccttggtttttagtagttttactatgatgaggtgtgccctccaccccatcatagtaaactactgaaaatcaaagatacaagtgcctgacca

SEQ ID NO: 301 - RNA, 91 nt, synthetic construct
  source 1..91: mol_type = other RNA
cgacttgctttctctcctccatgccttgagtgtaggaccgttggcatcttaattaccctcccacacccaaggcttgcagaagagcgagcct

SEQ ID NO: 302 - RNA, 94 nt, synthetic construct
  source 1..94: mol_type = other RNA
gtgaggtgtgggcccggccccaggagcggggcctgggcagccccgtgtgttgaggaaggaaggcagggcccccgctccccgggcctgaccccac

SEQ ID NO: 303 - RNA, 99 nt, synthetic construct
  source 1..99: mol_type = other RNA
aggtgggaggattgcttgagcctgggagatggaggttgcagtgagctgagatcacgcaactgcacccccagcctgggccatagagtcagtccttgtctc

SEQ ID NO: 304 - RNA, 19 nt, synthetic construct
  source 1..19: mol_type = other RNA
ctgaggtgaaacatcccag

SEQ ID NO: 305 - RNA, 19 nt, synthetic construct
  source 1..19: mol_type = other RNA
ctcaggtgaaacatccgag

SEQ ID NO: 306 - RNA, 19 nt, synthetic construct
  source 1..19: mol_type = other RNA
ctgaggtgaaacatctcag

SEQ ID NO: 307 - RNA, 78 nt, synthetic construct
  source 1..78: mol_type = other RNA
ctgaggagcagggcttagctgcttgtgagcagggtccacaccaagtcgtgttcacagtggctaagttccgctcctcag

SEQ ID NO: 308 - RNA, 73 nt, synthetic construct
  source 1..73: mol_type = other RNA
ggccggctggggttcctggggatgggatttgcttcttgtcacaaatcacattgccagggatttccaaccgacc

SEQ ID NO: 309 - RNA, 73 nt, synthetic construct
  source 1..73: mol_type = other RNA
ctctgcctcccgtgcctactgagctgaaacacagttggtttgtgtacactggctcagttcagcaggaacaggg

SEQ ID NO: 310 - DNA, 10 bp, synthetic construct
  source 1..10: mol_type = other DNA
cagcatctag